(19) United States
(12) Patent Application Publication
(10) Pub. No.: US 2024/0384315 A1
(43) Pub. Date: Nov. 21, 2024

(54) METHODS OF PRODUCING HUMAN MILK OLIGOSACCHARIDES AND COMPOSITIONS THEREOF

(71) Applicant: Amyris, Inc., Emeryville, CA (US)

(72) Inventors: Dominic PINEL, Emeryville, CA (US); Victoria HSIAO, Emeryville, CA (US); Diana Bahraini MARINA, Emeryville, CA (US); Derek A. ABBOTT, Emeryville, CA (US)

(21) Appl. No.: 18/688,617

(22) PCT Filed: Sep. 2, 2022
(86) PCT No.: PCT/US2022/075915

§ 371 Date: Mar. 1, 2024

Related U.S. Application Data
(60) Provisional application No. 63/240,245, filed on Sep. 2, 2021.

(51) Int. Cl. C12P 19/00; A23L 33/00; C12N 9/04; C12N 9/10; C12N 9/12; C12N 9/22; C12N 9/88; C12N 15/11; C12N 15/113; C12N 15/81

(52) U.S. Cl. CPC C12P 19/00; A23L 33/40; C12N 9/0006; C12N 9/1051; C12N 9/1205; C12N 9/22; C12N 9/88; C12N 15/11; C12N 15/1137; C12N 15/81; C12Y 101/01271; C12Y 204/01038; C12Y 204/01069; C12Y 204/01149; C12Y 207/01; C12Y 402/01047; C12N 2310/20

(57) ABSTRACT

Provided herein are host cells capable of producing a human milk oligosaccharide (HMO), such as yeast cells that are deficient in expression or activity of an endogenous oxidoreductase. Also provided are fermentation compositions including the disclosed host cells, as well as related methods of producing and recovering HMOs generated by the host cells.

### SEQUENCE LISTING

The instant application contains a Sequence Listing which has been submitted electronically in XML format and is hereby incorporated by reference in its entirety. Said XML copy, created on Aug. 31, 2022, is named 51494-018WO2_Sequence_Listing_8_31_22 and is 24,933 bytes in size.

### BACKGROUND OF THE INVENTION

Human milk oligosaccharides (HMOs) are the third most abundant component of human milk, with only lactose and lipids present in higher concentrations. More than 200 different species of HMOs have been identified to date in human milk, including the naturally occurring tetra-saccharide lacto-n-neotetraose (LNnT), belonging to the group of non-fucosylated neutral HMOs, and 2′-fucosyllactose (2′-FL). There is growing evidence attributing various health benefits to these milk compounds. Exemplary benefits include the promotion of the growth of protective intestinal microbes such as bifidobacteria, an increase in protection from gastrointestinal infections, a strengthening of the immune system, and an improvement in cognitive development. Because HMOs are not found in other milk sources, such as cow or goat, the only source of HMOs has traditionally been mother's milk. In efforts to improve the nutritional value of infant formula and expand the use of HMOs for child and adult nutrition, there has been an increased interest in the synthetic production of these compounds.

Heterologous production of LNnT in yeast requires only three non-native enzyme s: a lactose permease (e.g., LAC12 from K. lactis) to import fed lactose, an LgtA (β-1,3-N-acetylglucosaminyltransferase) to convert lactose to LNT2, and an LgtB (β-1 4-galactosyltransferase) to convert LNT2 to LNnT. However, despite the simplicity of the pathway, the generation of unwanted byproducts (e.g., lactitol, LNnT-alditol, and/or 2′-fucosyllactitol), which both decreases product purity and reduces overall yield, is a major challenge. Heterologous production of 2′-fucosyllactose in yeast requires four non-native enzymes: lactose permease (e.g., Lac12 from K. lactis) to import fed lactose, a GDP-mannose 4,6-dehydratase, a GDP-L-fucose synthase, and an α-1,2-fucosyltransferase. Generation of unwanted byproducts (e.g., 2′-fucosyllactitol), which both decreases product purity and reduces overall yield, is also a major challenge. Therefore, there remains a need for improved methods that result in enhanced HMO production and fewer unwanted byproducts.

### SUMMARY OF THE INVENTION

The compositions and methods of the present disclosure can be used to produce a variety of human milk oligosaccharides (HMOs) in high overall yield while suppressing the formation of undesirable impurities. Particularly, using the compositions and methods described herein, one can utilize a modified host cell to produce a HMO selected from, for example, lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, and fucosyl-disialyllacto-N-hexaose (FDS-LNH) II, while simultaneously attenuating the formation of unwanted impurities.

To engineer a host cell (e.g., a yeast cell described herein) to produce one or more of the foregoing HMOs in high overall yield while suppressing byproduct formation, one may use the compositions and methods described herein to render the host cell deficient in expression of one or more genes encoding an endogenous oxidoreductase enzyme. For example, using the compositions and methods of the disclosure, one may modify a host cell (e.g., a yeast cell) so as to render the host cell deficient in expression of one or more genes encoding an endogenous aldose reductase enzyme. In some embodiments, for instance, the host cell may be modified so as to be deficient in expression of one or more of genes gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and adh1. The host cell may be rendered deficient in expression of one or more of such genes by way of a genetic modification, such as a deletion of the one or more genes from the host cell genome, and/or by way of an inhibitor of gene expression, such as by way of an interfering RNA modality described herein.

Additionally or alternatively, one may engineer a host cell (e.g., a yeast cell described herein) to produce one or more of the foregoing HMOs in high overall yield while suppressing byproduct formation by modifying the host cell so as to be deficient in activity of one or more endogenous oxidoreductase enzymes, such as one or more endogenous aldose reductase enzymes. For example, using the composition and methods of the disclosure, one may modify a host cell (e.g., a yeast cell) so as to render the host cell deficient in activity of one or more enzymes selected from GCY1, GRE3, ADH6, SFA1, YPR1, GRE2, ARA1, BDH1, BDH2, ADH4, SER3, AAD6, AAD16, ARI1, ADH5, SER33, NRE1, IRC24, IDP2, AAD14, ALD6, ALD3, and ADH1. The host cell may be rendered deficient in activity of one or more of such enzymes by way of an inhibitor of enzyme activity, such as by way of an inhibitor modality described herein.

In an aspect, the invention provides a host cell capable of producing a HMO, wherein the host cell is (i) deficient in expression of one or more genes (e.g., 1, 2, 3, 4, 5, 6, 7, 8, 9, 10, 11, 12, 13, 14, 15, 16, 17, 18, 19, 20, or more genes) encoding an endogenous oxidoreductase, (ii) deficient in activity of one or more endogenous oxidoreductase proteins (e.g., 1, 2, 3, 4, 5, 6, 7, 8, 9, 10, 11, 12, 13, 14, 15, 16, 17, 18, 19, 20, or more endogenous oxidoreductase proteins); and/or (iii) deficient in a level of a reduced form of a reducing sugar, such as lactitol, LNnT-alditol, and/or 2′-fucosyllactitol.

In some embodiments, the endogenous oxidoreductase is an endogenous aldose reductase.

In some embodiments, the host cell is deficient in expression of the one or more genes encoding an endogenous oxidoreductase.

In some embodiments, the one or more genes include gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1.

In some embodiments, the one or more genes include gcy1. In some embodiments, the one or more genes include gre3. In some embodiments, the one or more genes include gcy1 and gre3. In some embodiments, the one or more genes include adh6. In some embodiments, the one or more genes include sfa1.

In some embodiments, the host cell is deficient in expression of the one or more genes by virtue of comprising a deletion of the one or more genes from the host cell genome. In some embodiments, the deletion removes one or more nucleotides (e.g., 1, 5, 10, 20, 30, 40, 50,100,200, 300, 400, 500, 1,000, or more nucleotides) from the gene, thereby resulting in a missense mutation, nonsense mutation, or frameshift mutation in the gene. In some embodiments, the deletion removes the one or more genes from the host cell genome in their entirety.

In some embodiments, the host cell is deficient in expression of the one or more genes by virtue of comprising an inhibitor of expression of the one or more genes. In some embodiments, the inhibitor of expression of the one or more genes is an interfering RNA molecule. In some embodiments, the interfering RNA molecule is a short interfering RNA (siRNA), a short hairpin RNA (shRNA), a micro RNA (miRNA), or an antisense oligonucleotide (ASO).

In some embodiments, the host cell is deficient in expression of the one or more genes by virtue of comprising a nuclease that catalyzes cleavage of one or more phosphodiester bonds in the one or more genes. In some embodiments, the nuclease is a clustered regularly interspaced short palindromic repeats (CRISPR)-associated protein. In some embodiments, the CRISPR-associated protein is CRISPR associated protein 9 (Cas9) or CRISPR-associated protein 12a (Cas12a). In some embodiments, the nuclease is a transcription activator-like effector nuclease (TALEN), a meganuclease, or a zinc finger nuclease.

In some embodiments, the host cell is deficient in activity of the one or more endogenous oxidoreductase proteins.

In some embodiments, the one or more endogenous oxidoreductase proteins include GCY1, GRE3, ADH6, SFA1, YPR1, GRE2, ARA1, BDH1, BDH2, ADH4, SER3, AAD6, AAD16, ARI1, ADH5, SER33, NRE1, IRC24, IDP2, AAD14, ALD6, ALD3, and/or ADH1.

In some embodiments, the one or more endogenous oxidoreductase proteins include GCY1. In some embodiments, the one or more endogenous oxidoreductase proteins include GRE3. In some embodiments, the one or more endogenous oxidoreductase proteins include GCY1 and GRE3. In some embodiments, the one or more endogenous oxidoreductase proteins include ADH6. In some embodiments, the one or more endogenous oxidoreductase proteins include SFA1.

In some embodiments the host cell is deficient in activity of the one or more endogenous oxidoreductase proteins by virtue of including an inhibitor of the one or more oxidoreductase proteins, such as an antibody or antigen-binding fragment thereof that specifically binds and inhibits the oxidoreductase protein and/or a small molecule that binds and inhibits the oxidoreductase protein.

In some embodiments, the host cell is deficient in a level of lactitol, LNnT-alditol, and/or 2′-fucosyllactitol.

In some embodiments, the host cell includes one or more (e.g., 1, 2, 3, or more) heterologous nucleic acids that each, independently, encode one or more enzymes of the biosynthetic pathway of the HMO. In some embodiments, the one or more heterologous nucleic acids encoding one or more enzymes of the biosynthetic pathway of the HMO are integrated into the genome of the host cell. In some embodiments, the one or more heterologous nucleic acids encoding one or more enzymes of the biosynthetic pathway of the HMO are present within one or more plasmids.

In some embodiments, the HMO is a reducing sugar. In some embodiments, the HMO includes a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

In some embodiments, the HMO is LNnT. In some embodiments, the one or more enzymes of the biosynthetic pathway of the HMO include one or more of a β-1,3-N-acetylglucosaminyltransferase (LgtA), a β-1,4-galactosyltransferase (LgtB), and a UDP-N-acetylglucosamine diphosphorylase.

In some embodiments, the one or more enzymes of the biosynthetic pathway of the HMO include a LgtA. In some embodiments, the LgtA has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 1. In some embodiments, the LgtA has an amino acid sequence that is at least 90% identical (e.g., at least 91%, 92%, 93%, 94%, 95%, 96%, 97%, 98%, or 99% identical) to the amino acid sequence of SEQ ID NO: 1. In some embodiments, the LgtA has an amino acid sequence that is at least 95% identical (e.g., at least 96%, 97%, 98%, or 99% identical) to the amino acid sequence of SEQ ID NO: 1. In some embodiments, the LgtA has the amino acid sequence of SEQ ID NO: 1. In some embodiments, the LgtA includes one or more (e.g., 1, 2, 3, 4, 5, 6, 7,8, 9, 10, or more) amino acid substitutions or deletions relative to the amino acid sequence of SEQ ID NO: 1. In some embodiments, the LgtA has an amino acid sequence that is from about 85% to about 99.7% identical (e.g., about 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1. In some embodiments, the LgtA has an amino acid sequence that is from about 90% to about 99.7% identical (e.g., about 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1. In some embodiments, the LgtA has an amino acid sequence that is from about 95% to about 99.7% identical (e.g., about 95.5%, 96%, 96.5%, 97%, 97.5%, 98%, 98.5%, 99%, or 99.5% identical) to the amino acid sequence of SEQ ID NO: 1.

In some embodiments, the LgtA has an amino acid sequence that differs from the amino acid sequence of SEQ ID NO: 1 only by way of (i) the one or more amino acid substitutions or deletions and, optionally, (ii) one or more (e.g., 1, 2, 3, 4, 5, 6, 7, 8, 9, 10, or more) additional, conservative amino acid substitutions. In some embodiments, the LgtA has an amino acid sequence that differs from the amino acid sequence of SEQ ID NO: 1 only by way of one or more conservative amino acid substitutions and/or by way of one or more amino acid deletions.

In some embodiments, the LgtA has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NO: 2-13. In some embodiments, the LgtA has an amino acid sequence that is at least 90% identical (e.g., at least 91%, 92%, 93%, 94%, 95%, 96%, 97%, 98%, or 99% identical) to the amino acid sequence of any one of SEQ ID NO: 2-13. In some embodiments, the LgtA has an amino acid sequence that is at least 95% identical (e.g., at least 96%, 97%, 98%, or 99% identical) to the amino acid sequence of any one of SEQ ID NO: 2-13. In some embodiments, the LgtA has the amino acid sequence of any one of SEQ ID NO: 2-13.

In some embodiments, the one or more enzymes of the biosynthetic pathway of the HMO include a LgtB. In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 15. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 15. In some embodiments, the LgtB has an amino acid sequence that is at least 95% identical (e.g., at least 96%, 97%, 98%, or 99% identical) to the amino acid sequence of SEQ ID NO: 15. In some embodiments, the LgtB has the amino acid sequence of SEQ ID NO: 15.

In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 16. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 16. In some embodiments, the LgtB has an amino acid sequence that is at least 95% identical (e.g., at least 96%, 97%, 98%, or 99% identical) to the amino acid sequence of SEQ ID NO: 16. In some embodiments, the LgtB has the amino acid sequence of SEQ ID NO: 16.

In some embodiments, the HMO is 2′-FL. The one or more enzymes may include, for example, one or more of a GDP-mannose 4,6-dehydratase, a GDP-L-fucose synthase, an α-1,2-fucosyltransferase, and optionally a fucosidase.

In some embodiments, the one or more enzymes include a GDP-mannose 4,6-dehydratase. In some embodiments, the GDP-mannose 4,6-dehydratase has an amino acid sequence that is at least 85% identical to the amino acid sequence of SEQ ID NO: 18. In some embodiments, the GDP-mannose 4,6-dehydratase has an amino acid sequence that is at least 90% identical to the amino acid sequence of SEQ ID NO: 18. In some embodiments, the GDP-mannose 4,6-dehydratase has an amino acid sequence that is at least 95% identical to the amino acid sequence of SEQ ID NO: 18. In some embodiments, the GDP-mannose 4,6-dehydratase has the amino acid sequence of SEQ ID NO: 18.

In some embodiments, the one or more enzymes include a GDP-L-fucose synthase. In some embodiments, the GDP-L-fucose synthase has an amino acid sequence that is at least 85% identical to the amino acid sequence of SEQ ID NO: 19. In some embodiments, the GDP-L-fucose synthase has an amino acid sequence that is at least 90% identical to the amino acid sequence of SEQ ID NO: 19. In some embodiments, the GDP-L-fucose synthase has an amino acid sequence that is at least 95% identical to the amino acid sequence of SEQ ID NO: 19. In some embodiments, the GDP-L-fucose synthase has the amino acid sequence of SEQ ID NO: 19.

In some embodiments, the one or more enzymes include an α-1,2-fucosyltransferase. In some embodiments, the α-1,2-fucosyltransferase has an amino acid sequence that is at least 85% identical to the amino acid sequence of SEQ ID NO: 20. In some embodiments, the α-1 2-fucosyltransferase has an amino acid sequence that is at least 90% identical to the amino acid sequence of SEQ ID NO: 20. In some embodiments, the α-1,2-fucosyltransferase has an amino acid sequence that is at least 95% identical to the amino acid sequence of SEQ ID NO: 20. In some embodiments, the α-1,2-fucosyltransferase has the amino acid sequence of SEQ ID NO: 20.

In some embodiments, the one or more heterologous nucleic acids further encode a protein that transports lactose into the host cell. In some embodiments, the protein that transports lactose into the host cell is a lactose permease. In some embodiments, the lactose permease has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 14. In some embodiments, the lactose permease has an amino acid sequence that is at least 90% identical (e.g., at least 91%, 92%, 93%, 94%, 95%, 96%, 97%, 98%, or 99% identical) to the amino acid sequence of SEQ ID NO: 14. In some embodiments, the lactose permease has an amino acid sequence that is at least 95% identical (e.g., at least 96%, 97%, 98%, or 99% identical) to the amino acid sequence of SEQ ID NO: 14. In some embodiments, the lactose permease has the amino acid sequence SEQ ID NO: 14.

In some embodiments, the protein that transports lactose into the host cell is an active transporter. In some embodiments, expression of the one or more heterologous nucleic acids is driven by an inducible promoter or is negatively regulated by the activity of a promoter that is responsive to a small molecule.

In some embodiments, the host cell is a yeast cell. In some embodiments, the yeast cell is a Saccharomyces sp. cell or a Kluveromyces sp. cell. In some embodiments, the yeast cell is a Saccharomyces cerevisiae cell. In some embodiments, the yeast cell is a Kluveromyces marxianus cell.

In another aspect, the disclosure provides a method of producing a HMO including culturing a population of any one of the host cells described herein in a culture medium under conditions suitable for the host cells to produce the HMO. In some embodiments, the HMO is a reducing sugar. In some embodiments, the HMO includes a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II. In some embodiments, the HMO is LNnT. In some embodiments, the HMO is 2′-FL.

In another aspect, the disclosure provides a fermentation composition including (i) a population of host cells including any one of the host cells described herein and (ii) a culture medium including a HMO produced from the host cells. In some embodiments, the HMO is a reducing sugar. In some embodiments, the HMO includes a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II. In some embodiments, the HMO is LNnT. In some embodiments, the HMO is 2′-FL.

In another aspect, the disclosure provides a method of genetically modifying a host cell to produce a HMO including: (i) rendering the host cell deficient in expression of one or more genes encoding an endogenous oxidoreductase and (ii) introducing one or more heterologous nucleic acids that each, independently, encode one or more enzymes of the biosynthetic pathway of the HMO into the host cell. Alternatively, the method may include rendering the host cell deficient in expression of one or more genes encoding an endogenous oxidoreductase, wherein the host cell includes one or more heterologous nucleic acids that each, independently, encode one or more enzymes of the biosynthetic pathway of the HMO. Alternatively, the method may include introducing one or more heterologous nucleic acids that each, independently, encode one or more enzymes of the biosynthetic pathway of the HMO into the host cell, wherein the host cell has previously been rendered deficient in expression of one or more genes encoding an endogenous oxidoreductase. In each of the foregoing aspects, the one or more heterologous nucleic acids, together with the endogenous genes present in the host cell, collectively encode the entirety of the enzymes of the biosynthetic pathway of the HMO.

In some embodiments of any of the foregoing aspects, the endogenous oxidoreductase is an endogenous aldose reductase. In some embodiments, the one or more genes include gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1. In some embodiments, the one or more genes include gcy1. In some embodiments, the one or more genes include gre3. In some embodiments, the one or more genes include gcy1 and gre3. In some embodiments, the one or more genes include adh6. In some embodiments, the one or more genes include sfa1.

In some embodiments, the host cell is deficient in expression of the one or more genes by virtue of comprising a deletion of the one or more genes from the host cell genome. In some embodiments, the deletion removes one or more nucleotides from the gene, thereby resulting in a missense mutation, nonsense mutation, or frameshift mutation in the gene. In some embodiments, the deletion removes the one or more genes from the host cell genome in their entirety. In some embodiments, the host cell is deficient in expression of the one or more genes by virtue of comprising an inhibitor of expression of the one or more genes.

In some embodiments, the inhibitor of expression of the one or more genes is an interfering RNA molecule. In some embodiments, the interfering RNA molecule is a siRNA, a shRNA, a miRNA, or an ASO.

In some embodiments, the host cell is deficient in expression of the one or more genes by virtue of comprising a nuclease that catalyzes cleavage of one or more phosphodiester bonds in the one or more genes. In some embodiments, the nuclease is a CRISPR-associated protein. In some embodiments, the CRISPR-associated protein is Cas9 or Cas12a. In some embodiments, the nuclease is a TALEN, a meganuclease, or a zinc finger nuclease.

In some embodiments, the one or more heterologous nucleic acids encoding one or more enzymes of the biosynthetic pathway of the HMO are integrated into the genome of the host cell. In some embodiments, the one or more heterologous nucleic acids encoding one or more enzymes of the biosynthetic pathway of the HMO are present within one or more plasmids. In some embodiments, the HMO is a reducing sugar. In some embodiments, the HMO includes a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

In some embodiments, the HMO is LNnT. In some embodiments, the one or more enzymes of the biosynthetic pathway of the HMO include one or more of a LgtA, a LgtB, and a UDP-N-acetylglucosamine diphosphorylase.

In some embodiments, the one or more enzymes of the biosynthetic pathway of the HMO include a LgtA. In some embodiments, the LgtA has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 1. In some embodiments, the LgtA has an amino acid sequence that is at least 90% identical (e.g., at least 91%, 92%, 93%, 94%, 95%, 96%, 97%, 98%, or 99% identical) to the amino acid sequence of SEQ ID NO: 1. In some embodiments, the LgtA has an amino acid sequence that is at least 95% identical (e.g., at least 96%, 97%, 98%, or 99% identical) to the amino acid sequence of SEQ ID NO: 1. In some embodiments, the LgtA has the amino acid sequence of SEQ ID NO: 1. In some embodiments, the LgtA includes one or more (e.g., 1, 2, 3, 4, 5, 6, 7,8, 9, 10, or more) amino acid substitutions or deletions relative to the amino acid sequence of SEQ ID NO: 1. In some embodiments, the LgtA has an amino acid sequence that is from about 85% to about 99.7% identical (e.g., about 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1. In some embodiments, the LgtA has an amino acid sequence that is from about 90% to about 99.7% identical (e.g., about 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1. In some embodiments, the LgtA has an amino acid sequence that is from about 95% to about 99.7% identical (e.g., about 95.5%, 96%, 96.5%, 97%, 97.5%, 98%, 98.5%, 99%, or 99.5% identical) to the amino acid sequence of SEQ ID NO: 1.

In some embodiments, the LgtA has an amino acid sequence that differs from the amino acid sequence of SEQ ID NO: 1 only by way of (i) the one or more amino acid substitutions or deletions and, optionally, (ii) one or more (e.g., 1, 2, 3, 4, 5, 6, 7, 8, 9, 10, or more) additional, conservative amino acid substitutions. In some embodiments, the LgtA has an amino acid sequence that differs from the amino acid sequence of SEQ ID NO: 1 only by way of one or more conservative amino acid substitutions and/or by way of one or more amino acid deletions.

In some embodiments, the LgtA has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NO: 2-13. In some embodiments, the LgtA has an amino acid sequence that is at least 90% identical (e.g., at least 91%, 92%, 93%, 94%, 95%, 96%, 97%, 98%, or 99% identical) to the amino acid sequence of any one of SEQ ID NO: 2-13. In some embodiments, the LgtA has an amino acid sequence that is at least 95% identical (e.g., at least 96%, 97%, 98%, or 99% identical) to the amino acid sequence of any one of SEQ ID NO: 2-13. In some embodiments, the LgtA has the amino acid sequence of any one of SEQ ID NO: 2-13.

In some embodiments, the one or more enzymes of the biosynthetic pathway of the HMO include a LgtB. In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 15. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 15. In some embodiments, the LgtB has an amino acid sequence that is at least 95% identical (e.g., at least 96%, 97%, 98%, or 99% identical) to the amino acid sequence of SEQ ID NO: 15. In some embodiments, the LgtB has the amino acid sequence of SEQ ID NO: 15.

In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 16. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 16. In some embodiments, the LgtB has an amino acid sequence that is at least 95% identical (e.g., at least 96%, 97%, 98%, or 99% identical) to the amino acid sequence of SEQ ID NO: 16. In some embodiments, the LgtB has the amino acid sequence of SEQ ID NO: 16.

In some embodiments, the HMO is 2′-FL. The one or more heterologous nucleic acids may encode, for example, a GDP-mannose 4,6-dehydratase, a GDP-L-fucose synthase, and/or an α-1,2-fucosyltransferase, optionally in combination with a fucosidase.

In some embodiments, the one or more heterologous nucleic acids encode a GDP-mannose 4,6-dehydratase. In some embodiments, the GDP-mannose 4,6-dehydratase has an amino acid sequence that is at least 85% identical to the amino acid sequence of SEQ ID NO: 18. In some embodiments, the GDP-mannose 4,6-dehydratase has an amino acid sequence that is at least 90% identical to the amino acid sequence of SEQ ID NO: 18. In some embodiments, the GDP-mannose 4,6-dehydratase has an amino acid sequence that is at least 95% identical to the amino acid sequence of SEQ ID NO: 18. In some embodiments, the GDP-mannose 4,6-dehydratase has the amino acid sequence of SEQ ID NO: 18.

In some embodiments, the one or more heterologous nucleic acids encode a GDP-L-fucose synthase. In some embodiments, the GDP-L-fucose synthase has an amino acid sequence that is at least 85% identical to the amino acid sequence of SEQ ID NO: 19. In some embodiments, the GDP-L-fucose synthase has an amino acid sequence that is at least 90% identical to the amino acid sequence of SEQ ID NO: 19. In some embodiments, the GDP-L-fucose synthase has an amino acid sequence that is at least 95% identical to the amino acid sequence of SEQ ID NO: 19. In some embodiments, the GDP-L-fucose synthase has the amino acid sequence of SEQ ID NO: 19.

In some embodiments, the one or more heterologous nucleic acids encode an α-1,2-fucosyltransferase. In some embodiments, the α-1,2-fucosyltransferase has an amino acid sequence that is at least 85% identical to the amino acid sequence of SEQ ID NO: 20. In some embodiments, the α-1,2-fucosyltransferase has an amino acid sequence that is at least 90% identical to the amino acid sequence of SEQ ID NO: 20. In some embodiments, the α-1,2-fucosyltransferase has an amino acid sequence that is at least 95% identical to the amino acid sequence of SEQ ID NO: 20. In some embodiments, the α-1,2-fucosyltransferase has the amino acid sequence of SEQ ID NO: 20.

In some embodiments, the one or more heterologous nucleic acids further encode a protein that transports lactose into the host cell. In some embodiments, the protein that transports lactose into the host cell is a lactose permease. In some embodiments, the lactose permease has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 14. In some embodiments, the lactose permease has an amino acid sequence that is at least 90% identical (e.g., at least 91%, 92%, 93%, 94%, 95%, 96%, 97%, 98%, or 99% identical) to the amino acid sequence of SEQ ID NO: 14. In some embodiments, the lactose permease has an amino acid sequence that is at least 95% identical (e.g., at least 96%, 97%, 98%, or 99% identical) to the amino acid sequence of SEQ ID NO: 14. In some embodiments, the lactose permease has the amino acid sequence SEQ ID NO: 14.

In some embodiments, the protein that transports lactose into the host cell is an active transporter. In some embodiments, expression of the one or more heterologous nucleic acids is driven by an inducible promoter or is negatively regulated by the activity of a promoter that is responsive to a small molecule.

In some embodiments, the host cell is a yeast cell. In some embodiments, the yeast cell is a Saccharomyces sp. cell or a Kluveromyces sp. cell. In some embodiments, the yeast cell is a Saccharomyces cerevisiae cell. In some embodiments, the yeast cell is a Kluveromyces marxianus cell.

In another aspect, the invention provides a method of producing an infant formula including culturing any one of the host cells capable of producing a HMO described herein in a culture medium under conditions suitable for the host cells to produce the HMO, extracting the HMO, and formulating the HMO for administration to an infant.

In some embodiments, the HMO present within the infant formula is a reducing sugar. In some embodiments, the HMO includes a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

In some embodiments, the infant formula comprises LNnT. In some embodiments, the infant formula comprises 2′-FL.

In another aspect, the invention provides an infant formula produced by culturing any one of the host cells capable of producing a HMO of described herein in a culture medium, extracting the HMO, formulating the HMO for administration to an infant.

### Definitions

As used herein in the context of a protein of interest, the term “activity” refers to the biological functionality that is associated with a wild-type form of the protein. For example, in the context of an enzyme, the term “activity” may refer to the ability of an enzyme to catalyze the conversion of a substrate into a product. The activity of the enzyme may be measured, for example, by determining the amount of product in a chemical reaction after a certain period of time, and/or by determining the amount of substrate remaining in the reaction mixture after a certain period of time. The activity of the enzyme can also be measured by determining the amount of an unused co-factor (e.g., NAD+ or NADP+) of the reaction remaining in the reaction mixture after a certain period of time. The quantity of an unused co-factor may be detected, for example, by spectrophotometric methods and/or other methods known in the art or described herein.

As used herein, the terms “anneal” and “hybridize” are used interchangeably and refer to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another). The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the term “capable of producing” refers to a host cell that is genetically modified to express the enzyme(s) necessary for the production of a given compound in accordance with a biochemical pathway that produces the compound. For example, a host cell (e.g., a yeast cell) that is “capable of producing” a human milk oligosaccharide (HMO) is one that expresses the enzymes necessary for production of the HMO according to the HMO biosynthetic pathway.

As used herein, a host cell that is “deficient” in expression of a gene of interest is one that is modified so as to exhibit reduced expression of the gene relative to a wild-type cell of the same species and strain lacking the modification of the deficient cell. The modification may be one that stably reduces expression of the gene of interest, such as a deletion of the gene from the host cell genome. The term “deletion” in the context of a gene of interest (e.g., an oxidoreductase gene described herein) refers to a modification to the gene that reduces, or altogether eliminates, expression of a functional product of the gene (e.g., a functional oxidoreductase protein product). Exemplary “deletions” in this context are modifications that remove a gene of interest from the host cell genome in its entirety. Additional examples of deletions include modifications that do not remove the gene of interest from the genome in its entirety, but instead remove one or more nucleotides from the gene. Such modifications may result in missense mutations, for example, such that the gene product, upon transcription and translation, no longer contains one or more amino acids critical to the function of the wild-type gene product. Additional examples of such modifications are those that give rise to nonsense mutations in which the reading frame of the gene of interest is prematurely terminated by the presence of a stop codon, as well as those that give rise to frameshift mutations in which the reading frame of the gene of interest has been shifted in a manner that no longer permits transcription and translation of a functional gene product. In some embodiments, in lieu of containing a “deletion” of the gene of interest, the host cell may contain the operative gene of interest in its genome, but may be deficient in expression of the gene by virtue of a modification that reduces transcription of the gene into an RNA transcript, reduces translation of the RNA transcript into a protein product, and/or reduces the activity or stability of the RNA transcript or the stability of the protein product. Examples of such modifications include the presence of inhibitory compounds, such as interfering RNA molecules and other inhibitors of gene expression, that (i) suppress the production of an RNA transcript corresponding to the gene of interest, (ii) reduce the stability of an RNA transcript corresponding to the gene of interest, and/or (iii) actively degrade an RNA transcript corresponding to the gene of interest.

Similarly, a host cell that is “deficient” in activity of a protein of interest is one that is modified so as to exhibited reduced activity of the protein relative to a wild-type cell of the same species lacking the modification of the deficient cell. Exemplary modifications that may be used in conjunction with the compositions and methods of the disclosure to effectuate a deficiency in protein activity include the presence of an inhibitor of the target protein, such as the presence of an antibody, antigen-binding fragment thereof, or small molecule that specifically binds and inhibits activity of the target protein.

Moreover, a host cell that is “deficient” in a level of a saccharide (e.g., a HMO described herein) or a sugar-alditol (e.g., LNnT-alditol and/or 2′-fucosyllactitol) is one that is modified so as to produce a reduced quantity and/or concentration of the saccharide or sugar-alditol relative to a wild-type cell of the same species lacking the modification of the deficient cell.

As used herein in the context of a gene or expression thereof, the term “disrupt” means to prevent the formation of a functional gene product. A gene product is functional if it fulfills its normal (wild-type) function(s). Disruption of the gene prevents expression of a functional RNA transcript or protein encoded by the gene. Disruption of the gene may be accomplished by, for example, an insertion, deletion, or substitution of one or more bases in a nucleic acid sequence of the gene or a corresponding transcription regulatory element that is operably linked to the gene, such as a promoter, enhancer, or operator that regulates expression of the gene in vivo. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein in the context of a gene, the term “express” refers to any one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a cell, tissue sample, or subject can manifest, for example, as: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art).

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that includes a polynucleotide sequence encoding a polypeptide for use in the invention operably linked to a promoter, e.g., its native promoter, where the expression cassette is introduced into a heterologous microorganism. In some embodiments, an expression cassette includes a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refers to a set of at least two different coding sequences, where the coding sequences encode enzymes that catalyze different parts of a synthetic pathway to form a desired product (e.g., a HMO). In a genetic pathway, a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

The term “host cell” as used in the context of this disclosure refers to a microorganism, such as yeast, and includes an individual cell or cell culture including a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

The terms “human milk oligosaccharide” and “HMO” are used interchangeably herein to refer to a group of nearly 200 identified sugar molecules that are found as the third most abundant component in human breast milk. HMOs in human breast milk are a complex mixture of free, indigestible carbohydrates with many different biological roles, including promoting the development of a functional infant immune system. HMOs include, without limitation, lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, and fucosyl-disialyllacto-N-hexaose (FDS-LNH II), among others.

The terms “variant LgtA” and “variant β-1,3-N-acetylglucosaminyltransferase” refer to a polypeptide having at least one (e.g., 1, 2, 3, 4, 5, 6, 7, 8, 9, 10, or more) amino acids substitutions or deletions relative to a wild-type LgtA polypeptide (e.g., a wild-type LgtA polypeptide from N. meningitidis, the amino acid sequence of which is set forth in SEQ ID NO: 1). The LgtA polypeptide may be modified (e.g., by way of one or more of the amino acid substitutions or deletions described herein) to enhance its specificity for binding to, and catalyzing the glycosidation of, the enzyme's intended substrate in the biosynthetic pathway of a HMO relative to a longer-chain oligosaccharide.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present disclosure will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

Two sequences are “substantially identical” if two sequences have a specified percentage of amino acid residues or nucleotides that are the same (i.e. , 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using a sequence comparison algorithm or by manual alignment and visual inspection as described above. Optionally, the identity exists over a region that is at least about 50 nucleotides (or 20 amino acids) in length, or more preferably over a region that is 100 to 500 or 1000 or more nucleotides (or 50, 100, or 200 or more amino acids) in length.

Nucleic acid or protein sequences that are substantially identical to a reference sequence include “conservatively modified variants.” With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu, and lie; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. The following six groups each contain amino acids that further provide illustrative conservative substitutions for one another. 1) Ala, Ser, Thr; 2) Asp, Glu; 3) Asn, Gln; 4) Arg, Lys; 5) lie, Leu, Met, Val; and 6) Phe, Try, and Trp (see, e.g., Creighton, Proteins: Structures and Molecular Principles. 1984, New York: W.H. Freeman).

Accordingly, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1, below.

TABLE 1
Representative physicochemical properties
of naturally-occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non-polar | neutral | intermediate |
| Serine | Ser | S | polar | neutral | small |
| Threonine | Thp | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “promoter” refers to a synthetic or naturally-derived nucleic acid that is capable of activating, increasing, or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

The term “reducing sugar” refers to a saccharide that contains a free aldehyde functional group or that can tautomerize in solution (e.g., in aqueous solution) to form an aldehyde group. Some disaccharides, oligosaccharides, polysaccharides, and all monosaccharides are reducing sugars. The monosaccharides can categorized into two groups: (1) aldoses that contain a free aldehyde group and (2) ketoses containing a ketone group. Ketoses must tautomerize to aidoses before acting as a reducing agent. Reducing sugars can be readily identified by way of a Tollens' test. A Tollens' test may be used to differentiate reducing sugars from non-reducing sugars. In a Tollens' test, a Tollens' reagent including silver ions and aqueous ammonia is added to a solution including the sugar of interest. The sugar may be identified as a reducing sugar if silver metal precipitates upon addition of the Tollens' reagent to the sugar of interest. For example, in some embodiments, the reducing sugar is lactose. In some embodiments, the reduced form of the reducing sugar (e.g., lactose) is lactitol. In some embodiments, the reducing sugar is LNnT. In some embodiments, the reduced form of the reducing sugar (e.g., LNnT) is LNnT-alditol. In some embodiments, the reducing sugar is 2′-fucosyllactose. In some embodiments, the reduced form of the reducing sugar is 2′-fucosyllactitol.

As used herein, the term “heterologous” refers to what is not normally found in nature. The term “heterologous nucleic acid” refers to a nucleic acid not normally found in a given cell in nature. A heterologous nucleic acid can be: (a) foreign to its host cell, i.e., exogenous to the host cell such that a host cell does not naturally contain the nucleic acid; (b) naturally found in the host cell, i.e., endogenous or native to the host cell, but present at an unnatural quantity in the cell (i.e., greater or lesser quantity than naturally found in the host cell); (c) be naturally found in the host cell but positioned outside of its natural locus. A “heterologous” polypeptide refers to a polypeptide that is encoded by a “heterologous nucleic acid”. Thus, for example, a “heterologous” polypeptide may be naturally produced by a host cell but is encoded by a heterologous nucleic acid that has been introduced into the host cell by genetic engineering. For example, a “heterologous” polypeptide can include embodiments in which an endogenous polypeptide is produced by an expression construct and is overexpressed in the host cell compared to native levels of the polypeptide produced by the host cell.

As used herein, the terms “interfering ribonucleic acid” and “interfering RNA” refer to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; (ii) promoting the nuclease-mediated degradation of the RNA transcript; and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4:e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein, the term “introducing” in the context of a nucleic acid or protein in a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The term also encompasses integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “transformation” refers to a genetic alteration of a host cell resulting from the introduction of exogenous genetic material, e.g., nucleic acids, into the host cell.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion.

As used herein, the term “antibody” (Ab) refers to an immunoglobulin molecule that specifically binds to, or is immunologically reactive with, a particular antigen, and includes polyclonal, monoclonal, genetically engineered, and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rIgG, and scFv fragments. In some embodiments, two or more portions of an immunoglobulin molecule are covalently bound to one another, e.g., via an amide bond, a thioether bond, a carbon-carbon bond, a disulfide bridge, or by a linker, such as a linker described herein or known in the art. Antibodies also include antibody-like protein scaffolds, such as the tenth fibronectin type III domain (¹⁰Fn3), which contains BC, DE, and FG structural loops similar in structure and solvent accessibility to antibody complementarity-determining regions (CDRs). The tertiary structure of the ¹⁰Fn3 domain resembles that of the variable region of the IgG heavy chain, and one of skill in the art can graft, e.g., the CDRs of a reference antibody onto the fibronectin scaffold by replacing residues of the BC, DE, and FG loops of ¹⁰Fn3 with residues from the CDR-H1, CDR-H2, or CDR-H3 regions, respectively, of the reference antibody.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated CDR; and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “operably linked” refers to a functional linkage between nucleic acid sequences such that the sequences encode a desired function. For example, a coding sequence for a gene of interest is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in a host cell.

As used in this specification and the appended claims, the singular forms “a,” “an,” and “the” include plural reference unless the context clearly dictates otherwise.

As used herein, the term “about” is used herein to mean a value that is ±10% of the recited value.

As used herein, the term “gcy1” refers to the glycerol 2-dehydrogenase (NADP(+)) gene, including any native gcy1 gene from any microbial source, including yeast cells (e.g., Saccharomyces cerevisiae), unless otherwise indicated. The term encompasses “full-length,” unprocessed gcy1 as well as any form of gcy1 that results from processing in the cell. The term also encompasses naturally occurring variants of gcy1, e.g., splice variants or allelic variants. The nucleic acid sequence of an exemplary gcy1 gene is shown in NCBI Reference Sequence No. NP_014763.1. The amino acid sequence of an exemplary protein encoded by a gcy1 gene is shown in UNIPROT™ Accession No. P14065-1.

As used herein, the term “gre3” refers to the NADPH-dependent aldose reductase GRE3 gene, including any native gre3 gene from any microbial source, including yeast cells (e.g., Saccharomyces cerevisiae), unless otherwise indicated. The term encompasses “full-length,” unprocessed gre3 as well as any form of gre3 that results from processing in the cell. The term also encompasses naturally occurring variants of gre3, e.g., splice variants or allelic variants. The nucleic acid sequence of an exemplary gre3 gene is shown in NCBI Reference Sequence No. NP_011972.1. The amino acid sequence of an exemplary protein encoded by a gre3 gene is shown in UNIPROT™ Accession No. P38715-1.

As used herein, the term “adh6” refers to the NADP-dependent alcohol dehydrogenase 6 gene, including any native adh6 gene from any microbial source, including yeast cells (e.g., Saccharomyces cerevisiae), unless otherwise indicated. The term encompasses “full-length,” unprocessed adh6 as well as any form of adh6 that results from processing in the cell. The term also encompasses naturally occurring variants of adh6, e.g., splice variants or allelic variants. The nucleic acid sequence of an exemplary adh6 gene is shown in NCBI Reference Sequence No. NP_014051.3. The amino acid sequence of an exemplary protein encoded by an adh6 gene is shown in UNIPROT™ Accession No. Q04894-1.

As used herein, the term “sfa1” refers to the S-(hydroxymethyl)glutathione dehydrogenase gene, including any native sfa1 gene from any microbial source, including yeast cells (e.g., Saccharomyces cerevisiae), unless otherwise indicated. The term encompasses “full-length,” unprocessed sfa1 as well as any form of sfa1 that results from processing in the cell. The term also encompasses naturally occurring variants of sfa1, e.g., splice variants or allelic variants. The nucleic acid sequence of an exemplary sfa1 gene is shown in NCBI Reference Sequence No. NP_010113.1. The amino acid sequence of an exemplary protein encoded by a sfa1 gene is shown in UNIPROT™ Accession No. P32771-1.

As used herein, the term “ypr1” refers to the putative reductase 1 gene of Saccharomyces cerevisiae, also referred to herein as NADPH-dependent aldo-keto reductase. The term encompasses “full-length,” unprocessed ypr1 as well as any form of ypr1 that results from processing in the cell. The term also encompasses naturally occurring variants of ypr1, e.g., splice variants or allelic variants. The nucleic acid sequence of an exemplary ypr1 gene is shown in NCBI Reference Sequence No. NP_010656.1. The amino acid sequence of an exemplary protein encoded by a ypr1 gene is shown in UNIPROT™ Accession No. Q12458-1.

As used herein, the term “gre2′ refers to the NADPH-dependent methylglyoxal reductase GRE2 gene, including any native gre2 gene from any microbial source, including yeast cells (e.g., Saccharomyces cerevisiae), unless otherwise indicated. The term encompasses “full-length,” unprocessed gre2 as well as any form of gre2 that results from processing in the cell. The term also encompasses naturally occurring variants of gre2, e.g., splice variants or allelic variants. The nucleic acid sequence of an exemplary gre2gene is shown in NCBI Reference Sequence No. NP_014490.1. The amino acid sequence of an exemplary protein encoded by a gre2gene is shown in UNIPROT™ Accession No. Q12068-1.

As used herein, the term “ara1” refers to the D-arabinose dehydrogenase [NAD(P)+] heavy chain gene, including any native ara1 gene from any microbial source, including yeast cells (e.g., Saccharomyces cerevisiae), unless otherwise indicated. The term encompasses “full-length,” unprocessed ara1 as well as any form of ara1 that results from processing in the cell. The term also encompasses naturally occurring variants of ara1, e.g., splice variants or allelic variants. The nucleic acid sequence of an exemplary ara1 gene is shown in NCBI Reference Sequence No. NP_009707.3. The amino acid sequence of an exemplary protein encoded by an ara1 gene is shown in UNIPROT™ Accession No. P38115-1.

As used herein, the term “bdh1” refers to the (R, R)-butanediol dehydrogenase gene, including any native bdh1 gene from any microbial source, including yeast cells (e.g., Saccharomyces cerevisiae), unless otherwise indicated. The term encompasses “full-length,” unprocessed bdh1 as well as any form of bdh1 that results from processing in the cell. The term also encompasses naturally occurring variants of bdh1, e.g., splice variants or allelic variants. The nucleic acid sequence of an exemplary bdh1 gene is shown in NCBI Reference Sequence No. NP_009341.2. The amino acid sequence of an exemplary protein encoded by a bdh1 gene is shown in UNIPROT™ Accession No. P39714-1.

As used herein, the term “bdh2′ refers to the diacetyl reductase [(R)-acetoin forming] 2 gene, including any native bdh2 gene from any microbial source, including yeast cells (e.g., Saccharomyces cerevisiae), unless otherwise indicated. The term encompasses “full-length,” unprocessed bdh2 as well as any form of bdh2 that results from processing in the cell. The term also encompasses naturally occurring variants of bdh2, e.g., splice variants or allelic variants. The nucleic acid sequence of an exemplary bdh2gene is shown in NCBI Reference Sequence No. NP_009340.1. The amino acid sequence of an exemplary protein encoded by a bdh2gene is shown in UNIPROT™ Accession No. P39713-1.

As used herein, the term “adh4” refers to the alcohol dehydrogenase 4 gene, including any native adh4 gene from any microbial source, including yeast cells (e.g., Saccharomyces cerevisiae), unless otherwise indicated. The term encompasses “full-length,” unprocessed adh4 as well as any form of adh4 that results from processing in the cell. The term also encompasses naturally occurring variants of adh4, e.g., splice variants or allelic variants. The nucleic acid sequence of an exemplary adh4 gene is shown in NCBI Reference Sequence No. NP_011258.2. The amino acid sequence of an exemplary protein encoded by an adh4 gene is shown in UNIPROT™ Accession No. P10127-1.

As used herein, the term “ser3” refers to the D-3-phosphoglycerate dehydrogenase 1 gene, including any native ser3 gene from any microbial source, including yeast cells (e.g., Saccharomyces cerevisiae), unless otherwise indicated. The term encompasses “full-length,” unprocessed ser3 as well as any form of ser3 that results from processing in the cell. The term also encompasses naturally occurring variants of ser3, e.g., splice variants or allelic variants. The nucleic acid sequence of an exemplary ser3 gene is shown in NCBI Reference Sequence No. NP_011004.3. The amino acid sequence of an exemplary protein encoded by a ser3 gene is shown in UNIPROT™ Accession No. P40054-1.

As used herein, the term “aad6” refers to the putative aryl-alcohol dehydrogenase AAD6 gene, including any native aad6 gene from any microbial source, including yeast cells (e.g., Saccharomyces cerevisiae), unless otherwise indicated. The term encompasses “full-length,” unprocessed aad6 as well as any form of aad6 that results from processing in the cell. The term also encompasses naturally occurring variants of aad6, e.g., splice variants or allelic variants. The nucleic acid sequence of an exemplary aad6 gene is shown in European Nucleotide Archive Accession No. AY693161.1. The amino acid sequence of an exemplary protein encoded by an aad6 gene is shown in UNIPROT™ Accession No. P43547-1.

As used herein, the term “aad16′ refers to the putative aryl-alcohol dehydrogenase AAD16 gene, including any native aad16 gene from any microbial source, including yeast cells (e.g., Saccharomyces cerevisiae), unless otherwise indicated. The term encompasses “full-length,” unprocessed aad16 as well as any form of aad16 that results from processing in the cell. The term also encompasses naturally occurring variants of aad16, e.g., splice variants or allelic variants. The nucleic acid sequence of an exemplary aad16 gene is shown in NCBI Reference Sequence No. NP_015237.1. The amino acid sequence of an exemplary protein encoded by an aad16 gene is shown in UNIPROT™ Accession No. Q02895-1.

As used herein, the term “aril” refers to the NADPH-dependent aldehyde reductase ARI1 gene, including any native ari1 gene from any microbial source, including yeast cells (e.g., Saccharomyces cerevisiae), unless otherwise indicated. The term encompasses “full-length,” unprocessed ari1 as well as any form of ari1 that results from processing in the cell. The term also encompasses naturally occurring variants of ari1, e.g., splice variants or allelic variants. The nucleic acid sequence of an exemplary aril gene is shown in NCBI Reference Sequence No. NP_011358.3. The amino acid sequence of an exemplary protein encoded by an ari1 gene is shown in UNIPROT™ Accession No. P53111-1.

As used herein, the term “adh5′ refers to the alcohol dehydrogenase 5 gene, including any native adh5 gene from any microbial source, including yeast cells (e.g., Saccharomyces cerevisiae), unless otherwise indicated. The term encompasses “full-length,” unprocessed adh5 as well as any form of adh5 that results from processing in the cell. The term also encompasses naturally occurring variants of adh5, e.g., splice variants or allelic variants. The nucleic acid sequence of an exemplary adh5 gene is shown in NCBI Reference Sequence No. NP_009703.3. The amino acid sequence of an exemplary protein encoded by an adh5 gene is shown in UNIPROT™ Accession No. P38113-1.

As used herein, the term “ser33” refers to the D-3-phosphoglycerate dehydrogenase 2 gene, including any native ser33 gene from any microbial source, including yeast cells (e.g., Saccharomyces cerevisiae), unless otherwise indicated. The term encompasses “full-length,” unprocessed ser33 as well as any form of ser33 that results from processing in the cell. The term also encompasses naturally occurring variants of ser33, e.g., splice variants or allelic variants. The nucleic acid sequence of an exemplary ser33 gene is shown in NCBI Reference Sequence No. NP_012191.1. The amino acid sequence of an exemplary protein encoded by a ser33 gene is shown in UNIPROT™ Accession No. P40510-1.

As used herein, the term “irc24” refers to the benzil reductase ((S)-benzoin forming) IRC24 gene, including any native irc24 gene from any microbial source, including yeast cells (e.g., Saccharomyces cerevisiae), unless otherwise indicated. The term encompasses “full-length,” unprocessed irc24 as well as any form of irc24 that results from processing in the cell. The term also encompasses naturally occurring variants of irc24, e.g., splice variants or allelic variants. The nucleic acid sequence of an exemplary irc24 gene is shown in NCBI Reference Sequence No. NP_012302.3. The amino acid sequence of an exemplary protein encoded by an irc24 gene is shown in UNIPROT™ Accession No. P40580-1.

As used herein, the term “idp2′ refers to the isocitrate dehydrogenase [NADP] cytoplasmic gene, including any native idp2 gene from any microbial source, including yeast cells (e.g., Saccharomyces cerevisiae), unless otherwise indicated. The term encompasses “full-length,” unprocessed idp2 as well as any form of idp2 that results from processing in the cell. The term also encompasses naturally occurring variants of idp2, e.g., splice variants or allelic variants. The nucleic acid sequence of an exemplary idp2 gene is shown in NCBI Reference Sequence No. NP_013275.1. The amino acid sequence of an exemplary protein encoded by an idp2gene is shown in UNIPROT™ Accession No. P41939-1.

As used herein, the term “aad14” refers to the putative aryl-alcohol dehydrogenase AAD14 gene, including any native aad14 gene from any microbial source, including yeast cells (e.g., Saccharomyces cerevisiae), unless otherwise indicated. The term encompasses “full-length,” unprocessed aad14 as well as any form of aad14 that results from processing in the cell. The term also encompasses naturally occurring variants of aad14, e.g., splice variants or allelic variants. The nucleic acid sequence of an exemplary aad14 gene is shown in NCBI Reference Sequence No. NP_014068.1. The amino acid sequence of an exemplary protein encoded by an aad14 gene is shown in UNIPROT™ Accession No. P42884-1.

As used herein, the term “ald6′ refers to the magnesium-activated aldehyde dehydrogenase, cytosolic gene, including any native ald6 gene from any microbial source, including yeast cells (e.g., Saccharomyces cerevisiae), unless otherwise indicated. The term encompasses “full-length,” unprocessed ald6 as well as any form of ald6 that results from processing in the cell. The term also encompasses naturally occurring variants of ald6, e.g., splice variants or allelic variants. The nucleic acid sequence of an exemplary ald6 gene is shown in NCBI Reference Sequence No. NP_015264.1. The amino acid sequence of an exemplary protein encoded by an ald6 gene is shown in UNIPROT™ Accession No. P54115-1.

As used herein, the term “ald3” refers to the aldehyde dehydrogenase [NAD(P)+] 2 gene, cytosolic gene, including any native ald3 gene from any microbial source, including yeast cells (e.g., Saccharomyces cerevisiae), unless otherwise indicated. The term encompasses “full-length,” unprocessed ald3 as well as any form of ald3 that results from processing in the cell. The term also encompasses naturally occurring variants of ald3, e.g., splice variants or allelic variants. The nucleic acid sequence of an exemplary ald3 gene is shown in NCBI Reference Sequence No. NP_013892.1. The amino acid sequence of an exemplary protein encoded by an ald3 gene is shown in UNIPROT™ Accession No. P54114-1.

As used herein, the term “adh1” refers to the alcohol dehydrogenase 1 gene, cytosolic gene, including any native adh1 gene from any microbial source, including yeast cells (e.g., Saccharomyces cerevisiae), unless otherwise indicated. The term encompasses “full-length,” unprocessed adh1 as well as any form of adh1 that results from processing in the cell. The term also encompasses naturally occurring variants of adh1, e.g., splice variants or allelic variants. The nucleic acid sequence of an exemplary adh1 gene is shown in NCBI Reference Sequence No. NP_014555.1. The amino acid sequence of an exemplary protein encoded by an adh1 gene is shown in UNIPROT™ Accession No. P00330-1.

As used herein, the term “nre1” refers to the putative cytoplasmic short-chain dehydrogenase/reductase gene, located between residues 421026 and 421790 of Saccharomyces cerevisiae chromosome IX, including any native nre1 gene from any other microbial source, unless otherwise indicated. The term encompasses “full-length,” unprocessed nre1 as well as any form of nre1 that results from processing in the cell. The term also encompasses naturally occurring variants of nre1, e.g., splice variants or allelic variants.

### BRIEF DESCRIPTION OF DRAWINGS

FIG. 1 is a graph showing an exemplary ion chromatography product profile from top lacto-N-neotetraose (LNnT)-producing strain Y67002.

FIG. 2A is a schematic showing the biosynthetic pathway for producing LNnT and the conversion of LNnT pathway intermediates to sugar alditols by unknown native yeast reductases. Conversion of pathway products into sugar alditols reduces overall yield of LNnT.

FIG. 2B is a schematic showing the biosynthetic pathway for producing 2′-FL. 2′-FL is converted to 2′-fucosyllactitol by unknown native yeast reductases, reducing overall yield of 2′-FL.

FIG. 3A is a schematic drawing showing the chemical structure of LNnT.

FIG. 3B is a schematic drawing showing the chemical structure of LNnT-alditol.

FIG. 4 is a graph showing GCY1 and YPR1 are hits for enzymes that reduce lactose to lactitol. The graph shows that the gcy1Δ strain has no detectable lactitol and the ypr1A strain reduces median lactitol by 2-3-fold relative to unmodified parent cells.

FIG. 5 is a graph showing the measured biomass (using absorbance/optical density (SSOD)) of reductase knockout strains.

FIG. 6 is a series of graphs showing performance of two strains, Y67002 (Parent) and Y68726 (gcy1A), in producing LNnT.

FIG. 7 is a graph showing biomass (SSOD) measurements from double reductase deletion strains and gcy1Δ parent.

FIG. 8 is a graph showing stacked relative titer measurements for double reductase deletion strains compared to parent gcy1Δ strain. Each vertical bar in the graph shows, from bottom to top, the following quantities: relative LNnT titer, relative LNT2 titer, relative p-LNnH titer, and relative lactose titer.

FIG. 9 is a graph showing that peak area analysis of Unknown A2 demonstrates significant reduction from additional gre3 deletion in plate model (0.1% lactose).

FIG. 10 is a series of graphs showing bench-scale fermentation purity data for Y70141 strain (gcy1A, gre3A) compared to strains Y68726 (gcy1A) and Y67002.

FIG. 11 is a series of graphs showing peak area analysis for Y70141 strain (gcy1A, gre3A) compared to strains Y68726 (gcy1A) and Y67002.

FIG. 12A is a graph showing an ion chromatography trace for Y67002. Lactitol and Unknown A2 peaks are indicated by the black arrows.

FIG. 12B is a graph showing an ion chromatography trace for Y68726. Lactitol and Unknown A2 peaks are indicated by the black arrows.

FIG. 12C is a graph showing an ion exchange chromatography (IC) trace for Y70141. Lactitol and Unknown A2 peaks are indicated by the black arrows.

FIG. 13 is a graph showing an overlay of IC chromatograms of the double-deletion parent and the triple deletion strain with adh6Δ.

FIG. 14 is a graph showing an overlay of IC chromatograms of the double-deletion parent (black) and the triple deletion strain with sfa1Δ. The triple deletion strain has significant reduction in an unknown peak at 3.9 minutes (which is likely to be LNT2-alditol) and LNT2 (arrows).

FIG. 15 is a graph showing peak area analysis for all reductase KO strains tested.

FIG. 16A is a graph showing an overlay of IC chromatograms from Y71152 (+adh6Δ) and Y67002.

FIG. 16B is a graph showing a leftward shift in the apex of Unknown A2 when Y71152 is compared to Y67002 (no reductase knockouts).

FIG. 17 is a graph an overlay of IC chromatograms from Y71148 (+sfa1Δ) with Y67002.

FIG. 18 is an IC overlay showing that LNnT-alditol is one of at least two peaks co-eluting in the Unknown A2 peak found only in Y67002 lineage and not Y62381.

FIG. 19 is a graph showing that knocking out gcy1 from Y71081 led to a 2.6 fold reduction in 2′-fucosyllactitol peak area compared to the parent strain.

FIG. 20 is a graph showing an overlay of IC chromatograms from Y71081 (parent strain) and the gcy1 knockout strain. Left panel is a full chromatogram from the parent strain Y71081 with the 2′-fucosyllactitol peak highlighted by the arrow. Compared to 2′-FL and lactose peaks, the 2′-fucosyllactitol peak is particularly small. Right panel is an enlarged overlay of chromatograms from the parent and three cPCR-confirmed clones of the gcy1 knockout. Compared to the parent, gcy1 knockout reduced the peak size of both the 2′-fucosyllactitol peak and the neighboring unlabeled peak. The chromatograms show a distinct reduction in peak size of 2′-fucosyllactitol in the gcy1 knockout compared to the parent strain.

FIG. 21 is a graph showing the relative titer data of the parent Y71081 strain and the gcy1 knockout strain. Deletion of gcy1 did not significantly affect 2′-FL concentration.

FIG. 22 is a graph showing biomass (SSOD) measurements from the parent Y71081 strain and the gcy1 knockout strain. Deletion of gcy1 did not significantly affect biomass measured by optical density (SSOD).

FIG. 23 includes two graphs showing that deletion of gcy1 in a 2′-FL producing strain reduces the amount of 2′-fucosyllactitol produced (bottom) without reducing 2′-FL titer (top) as measured by IC peak area.

### DETAILED DESCRIPTION

The present disclosure features host cells capable of producing one or more human milk oligosaccharides (HMOs), as well as methods of using such host cells to produce a HMO in high overall yield while simultaneously suppressing the formation of undesirable impurities. The host cells described herein may be, for example, deficient in expression of one or more genes encoding an endogenous oxidoreductase (e.g., an endogenous aldose reductase). Additionally or alternatively, the host cells of the disclosure may be deficient in activity of one or more endogenous oxidoreductase proteins (e.g., one or more endogenous aldose reductase proteins). And in still further aspects of the disclosure, the host cells may be deficient in a level of a reduced form of a reducing sugar, such as, for example, lactitol, lacto-N-neotetraose (LNnT)-alditol, and/or 2′-fucosyllactitol.

It has presently been discovered that host cells having a deficiency in the expression and/or activity of the one or more endogenous oxidoreductases described herein, and/or a deficiency in a level of a reduced form of a reducing sugar, are capable of producing a desired HMO with greater purity and overall yield relative to host cells that do not have such a deficiency. The following sections provide a detailed description of the modified host cells that may be used to produce a HMO with heightened overall yield and enhanced purity, as well as exemplary techniques for preparing such modified host cells.

Host Cells Deficient in Expression and/or Activity of an Endogenous Oxidoreductase

The host cells of the disclosure may be used to produce one or more of a variety of HMOs, including, without limitation, lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, and fucosyl-disialyllacto-N-hexaose (FDS-LNH) II. The host cells of the disclosure may be deficient in expression of one or more genes encoding an endogenous oxidoreductase, such as an endogenous aldose reductase.

For example, the host cells of the disclosure may be modified so as to be deficient in expression of one or more oxidoreductase genes, such as gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1, relative to a wild-type cell of the same species lacking the modification. In some embodiments, the host cells may be deficient in expression of gcy1. In some embodiments, the host cells may be deficient in expression of gre3. In some embodiments, the host cells may be deficient in expression of gcy1 and gre3. In some embodiments, the host cells may be deficient in expression of adh6. In some embodiments, the host cells may be deficient in expression of sfa1.

Additionally or alternatively, the host cells may be modified so as to be deficient in activity of one or more endogenous oxidoreductase proteins, such as GCY1, GRE3, ADH6, SFA1, YPR1, GRE2, ARA1, BDH1, BDH2, ADH4, SER3, AAD6, AAD16, ARI1, ADH5, SER33, NRE1, IRC24, IDP2, AAD14, ALD6, ALD3, and/or ADH1, relative to a wild-type cell of the same species lacking the modification. In some embodiments, the host cells may be deficient in activity of GCY1. In some embodiments, the host cells may be deficient in activity of GRE3. In some embodiments, the host cells may be deficient in activity of GCY1 and GRE3. In some embodiments, the host cells may be deficient in activity of ADH6. In some embodiments, the host cells may be deficient in activity of SFA1.

Additionally or alternatively, the host cells may be modified so as to be deficient in a level of a reduced form of a reducing sugar relative to a wild-type cell of the same species lacking the modification. For example, the host cells may have deficient levels of lactitol. In some embodiments, the host cells may have deficient levels of LNnT-alditol. In some embodiments, the host cells may have deficient levels of 2′-fucosyllactitol.

Methods of Reducing Expression and/or Activity of an Endogenous Oxidoreductase

Also provided herein are methods genetically modifying a host cell to produce a cell that is capable of synthesizing a desired HMO in heightened overall yield and purity. The method may include rendering the host cell deficient in expression of one or more genes encoding an endogenous oxidoreductase (e.g., one or more of gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1) and introducing one or more heterologous nucleic acids that each, independently, encode one or more enzymes of the biosynthetic pathway of the HMO into the host cell, wherein the one or more heterologous nucleic acids, together with the endogenous genes present in the host cell, collectively encode the entirety of the enzymes of the biosynthetic pathway of the HMO.

Alternatively, the method may include rendering the host cell deficient in expression of one or more genes encoding an endogenous oxidoreductase (e.g., one or more of gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1), wherein the host cell includes one or more heterologous nucleic acids that each, independently, encode one or more enzymes of the biosynthetic pathway of the HMO, and wherein the one or more heterologous nucleic acids, together with the endogenous genes present in the host cell, collectively encode the entirety of the enzymes of the biosynthetic pathway of the HMO.

Alternatively, the method may include introducing one or more heterologous nucleic acids that each, independently, encode one or more enzymes of the biosynthetic pathway of the HMO into the host cell, wherein the host cell has previously been rendered deficient in expression of one or more genes encoding an endogenous oxidoreductase (e.g., gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1), and wherein the one or more heterologous nucleic acids, together with the endogenous genes present in the host cell, collectively encode the entirety of the enzymes of the biosynthetic pathway of the HMO.

The following sections describe exemplary techniques that may be used to modify a host cell so as to be deficient in expression of one or more genes encoding an endogenous oxidoreductase (e.g., one or more of gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1).

### Interfering RNA-Mediated Oxidoreductase Disruption

In some embodiments of the disclosure, a host cell may be modified so as to disrupt expression of an endogenous oxidoreductase gene. Exemplary methods for disrupting endogenous oxidoreductase gene expression include those in which an interfering RNA molecule is provided to host cell. The interfering RNA molecule may decrease the expression level (e.g., protein level or mRNA level) of an endogenous oxidoreductase. For example, interfering RNA molecules useful in conjunction with the compositions and methods of the disclosure include short interfering RNA (siRNA), short hairpin RNA (shRNA), and micro RNA (miRNA) molecules that target an endogenous oxidoreductase gene.

An exemplary siRNA is a double-stranded RNA molecule that typically has a length of from about 10 to 100 base pairs (e.g., 15 to 50 base pairs, such as 17 to 30 base pairs). An exemplary shRNA is a RNA molecule including a hairpin turn that decreases expression of target genes by way of RNA interference. shRNAs can be delivered to cells in the form of plasmids, such as viral vectors or other expression plasmids (e.g., by transfection, electroporation, or transduction). An exemplary miRNA is a non-coding RNA molecule that typically has a length of from about 10 to 100 nucleotides (e.g., 15 to 150 nucleotides). An miRNA may bind to a target site on a desired mRNA molecule (e.g., an mRNA molecule encoding an oxidoreductase protein) and silence the mRNA, e.g., by causing cleavage of the mRNA, destabilization of the mRNA, or inhibition of translation of the mRNA.

An interfering RNA molecule of the disclosure may contain, for example, chemically modified nucleotides, such as 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, certain modification can increase nuclease resistance and/or serum stability, or decrease immunogenicity.

In some embodiments, the inhibitory RNA molecule decreases the level and/or activity of an endogenous oxidoreductase in a host cell, for example, yeast cell. In some embodiments, the interfering RNA molecule inhibits expression of an endogenous oxidoreductase. In some embodiments, the interfering RNA molecule increases degradation of endogenous oxidoreductase mRNA and/or decreases the stability of endogenous oxidoreductase mRNA. The interfering RNA molecule can be chemically synthesized or transcribed in vitro.

The synthesis and use of interfering RNA molecules is described, for example, in Sioud, RNA Therapeutics: Function, Design, and Delivery (Methods in Molecular Biology). Humana Press 2010.

### Nuclease-Mediated Endogenous Oxidoreductase Disruption

Another useful tool for the disruption of a target gene, including gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1, involves the use of a nuclease, such as a clustered regularly interspaced short palindromic repeats (CRISPR)/Cas system. CRISPR/Cas originally evolved as an adaptive defense mechanism in bacteria and archaea against viral infection. A CRISPR/Cas system includes palindromic repeat sequences within plasmid DNA and a CRISPR-associated protein (Cas; e.g., Cas9 or Cas12a). This ensemble of DNA and protein directs site specific DNA or RNA (e.g., mRNA) cleavage of a target sequence by first incorporating foreign DNA into CRISPR loci. Polynucleotides containing these foreign sequences and the repeat-spacer elements of the CRISPR locus are in turn provided to (e.g., transcribed in) a host cell to create a guide RNA, which can subsequently anneal to a target sequence (e.g., in DNA or RNA, such as mRNA) and localize the Cas nuclease to this site. In this manner, highly site-specific Cas-mediated DNA or RNA (e.g., mRNA) cleavage can be engendered in a target polynucleotide (e.g., a polynucleotide encoding an oxidoreductase) because the interaction that brings Cas within close proximity of the target DNA molecule is governed by RNA:DNA or RNA:RNA hybridization. As a result, one can design a CRISPR/Cas system to cleave a target DNA or RNA molecule of interest (e.g., an endogenous oxidoreductase gene, e.g., gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1). This technique has been exploited in order to edit eukaryotic genomes (Hwang et al. Nature Biotechnology 31:227 (2013), the disclosure of which is incorporated herein by reference) and can be used as an efficient means of site-specifically editing a host cell (e.g., a yeast cell), in order to cleave DNA or RNA (e.g., mRNA) corresponding to an endogenous oxidoreductase gene, including gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1 gene. The use of CRISPR/Cas to modulate gene expression has been described in, e.g., U.S. Pat. No. 8,697,359, the disclosure of which is incorporated herein by reference.

Additional nuclease-driven methods for disruption of a target DNA include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (201 O); and in Joung et al. Nature Reviews Molecular Cell Biology 14:49 (2013), the disclosures of each of which are incorporated herein by reference.

miRNA Targeting Regions

An additional example of a technique that may be used to silence an oxidoreductase gene, such as an oxidoreductase gene selected from gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and adh1, involves the use of miRNA targeting regions. For example, a miRNA targeting region having complementarity to a miRNA expressed in host cell (e.g., a yeast cell) may be incorporated into a gene locus (e.g., using molecular biology techniques known in the art or described herein). The introduced miRNA targeting region can induce negative regulation of the neighboring gene. For example, upon transcription of an endogenous oxidoreductase gene (such as gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1) and the adjacent miRNA targeting sequence in a host cell, the miRNA targeting sequence can be recognized by, and may anneal to, a miRNA expressed in the host cell. The miRNA may, in turn, induce degradation and/or destabilization of the endogenous oxidoreductase mRNA, resulting in a reduction in expression and/or activity of the endogenous oxidoreductase.

In some embodiments, host cells of the disclosure are deficient in expression of a gene encoding an endogenous oxidoreductase, such as gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1 by virtue of containing an oxidoreductase gene cassette in which the nucleic acid encoding the oxidoreductase is operably linked to a miRNA targeting region that is specific for a miRNA expressed by the host cell.

### Antibody-Mediated Inhibition and Degradation of Endogenous Oxidoreductase

An exemplary method that can be used to suppress activity of an endogenous oxidoreductase protein, such as GCY1, GRE3, ADH6, SFA1, YPR1, GRE2, ARA1, BDH1, BDH2, ADH4, SER3, AAD6, AAD16, ARI1, ADH5, SER33, NRE1, IRC24, IDP2, AAD14, ALD6, ALD3, and/or ADH1, involves modifying the cell such that it contains (e.g., expresses) an anti-oxidoreductase antibody, or antigen-binding fragment thereof. The antibody or antigen-binding fragment thereof may inhibit oxidoreductase protein activity and/or promote oxidoreductase protein degradation. In some embodiments, the antibody or antigen-binding fragment thereof sequesters the oxidoreductase in a location (e.g., a sub-cellular organelle) such that the oxidoreductase is unable to perform its wild-type catalytic function. The antibody or antigen-binding fragment thereof may be, for example, a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, or a tandem scFv (taFv). In some embodiments, the antibody or antigen-binding fragment thereof is an scFv.

### Transcription Repressors and Dominant Negative Mutants

Additional examples of techniques that can be used to attenuate expression of an oxidoreductase gene (e.g., gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1) in a host cell involve modifying the cell so as to contain or overexpress a transcription repressor of the endogenous oxidoreductase gene.

Further examples of techniques that can be used to attenuate expression of an endogenous oxidoreductase gene (e.g., gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1) in a host cell involve modifying the cell so as to contain a dominant negative mutant form of the oxidoreductase protein or a dominant negative mutant form of a transcription activator specific for the oxidoreductase. Examples of dominant negative mutations in transcription factors that modulate protein expression are described, e.g., in Ohta et al., Journal of Molecular Medicine 89:43-50 (2011), the disclosure of which is incorporated herein by reference.

### Modulation of Signaling

In some embodiments, expression and/or activity of an endogenous oxidoreductase may be reduced in a host cell by virtue of the cell containing (e.g., expressing) an inhibitor of one or more components of a oxidoreductase signaling pathway. The cell may be, for example, deficient in expression and/or activity of the endogenous oxidoreductase by virtue of containing (e.g., expressing) an inhibitor of a transcription activator that is responsible for promoting oxidoreductase transcription. In some embodiments, the cell is deficient in expression and/or activity of the endogenous oxidoreductase by virtue of the cell containing (e.g., expressing) an inhibitor of a downstream component of an oxidoreductase signaling pathway.

### Genetic Polymorphisms

Additional examples of techniques that can be used to attenuate expression and/or activity in a cell of an endogenous oxidoreductase include modifying the cell such that it contains (e.g., expresses) a variant oxidoreductase gene (e.g., a variant of one or more of gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and adh1) containing a single nucleotide polymorphism (SNP) that is associated with a reduction in functional activity of the endogenous oxidoreductase.

### Enzymes of the Biosynthetic Pathway of a Target HMO

In addition to being modified so as to be deficient in expression and/or activity of one or more endogenous oxidoreductases (e.g., one or more endogenous aldose reductases described herein), host cells of the disclosure may also be modified so as to express the enzymes of the biosynthetic pathway of a target HMO. In some embodiments, for example, host cells of the disclosure (e.g., yeast cells) may naturally express some of the enzymes of the biosynthetic pathway for a given HMO. Such host cells may be modified to express the remaining enzymes of the biosynthetic pathway. In some embodiments, for instance, a host cell (e.g., a yeast cell) may naturally express many of the enzymes of the biosynthetic pathway of a desired HMO (e.g., LNnT, 2′-FL), and the host cells may be modified so as to express the remaining enzymes of the biosynthetic pathway for the desired HMO by providing the cells with one or more heterologous nucleic acid molecules that, together, encode the remaining enzymes of the biosynthetic pathway.

In some embodiments, host cells of the disclosure are provided with heterologous nucleic acid molecules that encode one or more enzymes of a pathway for synthesizing LNnT, including a β-1,3-N-acetylglucosaminyltransferase (LgtA), a β-1,4-galactosyltransferase (LgtB), and a UDP-N-acetylglucosamine diphosphorylase. Exemplary LgtA and LgtB enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing 2′-FL, including a lactose permease, a GDP-mannose 4,6-dehydratase, a GDP-L-fucose synthase, an α-1,2-fucosyltransferase, and a fucosidase. Exemplary enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing 3-fucosyllactose, including a GDP-mannose 4,6-dehydratase, a GDP-L-fucose synthase, an α-1,3-fucosyltransferase, and a fucosidase. Exemplary enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing lacto-N-tetraose, including a β-1,3-N-acetylglucosaminyltransferase, a β-1,3-galactosyltransferase, and a UDP-N-acetylglucosamine diphosphorylase. Exemplary enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing 3′-sialyllactose, including a CMP-Neu5Ac synthetase, a sialic acid synthase, a UDP-N-acetylglucosamine 2-epimerase, a UDP-N-acetylglucosamine diphosphorylase, and a CMP-N-acetylneuraminate-β-galactosamide-α-2,3-sialyltransferase. Exemplary enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing 6′-sialyllactose, including a CMP-Neu5Ac synthetase, a sialic acid synthase, a UDP-N-acetylglucosamine 2-epimerase, a UDP-N-acetylglucosamine diphosphorylase, and a β-galactoside-α-2,6-sialyltransferase. Exemplary enzymes useful in conjunction with the compositions and methods of the disclosure are described in the sections that follow.

In some embodiments, host cells of the disclosure are provided with heterologous nucleic acid molecules that encode one or more enzymes of a pathway for synthesizing difucosyllactose, including a GDP-mannose 4,6-dehydratase, a GDP-L-fucose synthase, an α-1,2-fucosyltransferase, and an α-1,3-fucosyltransferase. Exemplary enzymes useful in conjunction with the compositions and methods of the disclosure are described in the sections that follow.

### β-1,3-N-acetylglucosaminyltransferase Polypeptides

The LgtA polypeptides of the disclosure can be used to produce one or more of a variety of HMOs, including, without limitation, LNnT, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, LST a, LST b, LST c, DS-LNT, F-LST a, F-LST b, FS-LNH, FS-LNnH I, and FDS-LNH II.

In some embodiments, a LgtA polypeptide of the disclosure contains one or more amino acid substitutions relative to the wild-type LgtA amino acid sequence set forth in SEQ ID NO: 1. The amino acid substitution may occur, for example, at a residue selected from P89, G179, N180, 1182, H183, N185, T186, M187, W206, A207, Q211, W213, L229, V230, R233, H235, S240, K242, Y243, Q247, 1250, 1254, Q255, A258, L288, and E294 of SEQ ID NO: 1.

In some embodiments, the LgtA polypeptide includes an amino acid substitution at residue G179 of SEQ ID NO: 1. For example, the amino acid substitution at residue G179 of SEQ ID NO: 1 may substitute G179 with an amino acid including a cationic side chain at physiological pH. In some embodiments, the amino acid substitution at residue G179 of SEQ ID NO: 1 is a G179R substitution.

In some embodiments, the LgtA polypeptide includes an amino acid substitution at residue P89 of SEQ ID NO: 1. For example, the amino acid substitution at residue P89 of SEQ ID NO: 1 may substitute P89 with an amino acid including a polar, uncharged chain at physiological pH. In some embodiments, the amino acid substitution at residue P89 of SEQ ID NO: 1 is a P89T substitution.

In some embodiments, the LgtA polypeptide includes an amino acid substitution at residue N180 of SEQ ID NO: 1. For example, the amino acid substitution at residue N180 of SEQ ID NO: 1 may substitute N180 with an amino acid including an anionic side chain at physiological pH. In some embodiments, the amino acid substitution at residue N180 of SEQ ID NO: 1 is an N180D substitution. In some embodiments, the amino acid substitution at residue N180 of SEQ ID NO: 1 substitutes N180 with an amino acid including a hydrophobic, uncharged side chain at physiological pH. For example, the amino acid substitution at residue N180 of SEQ ID NO: 1 may be an N1 80A substitution.

In some embodiments, the LgtA polypeptide includes an amino acid substitution at residue I182 of SEQ ID NO: 1. In some embodiments, the amino acid substitution at residue I182 of SEQ ID NO: 1 substitutes I182 with an amino acid including a hydrophobic, uncharged side chain at physiological pH. For example, the amino acid substitution at residue I182 of SEQ ID NO: 1 may be an 11 82Y substitution.

In some embodiments, the LgtA polypeptide includes an amino acid substitution at residue H183 of SEQ ID NO: 1. For example, the amino acid substitution at residue H183 of SEQ ID NO: 1 may be an H183P substitution. In some embodiments, the amino acid substitution at residue H183 of SEQ ID NO: 1 substitutes H183 with an amino acid including a polar, uncharged side chain at physiological pH. For example, the amino acid substitution at residue H183 of SEQ ID NO: 1 may be an H1 83S substitution.

In some embodiments, the LgtA polypeptide includes an amino acid substitution at residue N185 of SEQ ID NO: 1. For example, the amino acid substitution at residue N185 of SEQ ID NO: 1 may be an N1 85G substitution.

In some embodiments, the LgtA polypeptide includes an amino acid substitution at residue T186 of SEQ ID NO: 1. In some embodiments, the amino acid substitution at residue T186 of SEQ ID NO: 1 substitutes T186 with an amino acid including an anionic side chain at physiological pH. For example, the amino acid substitution at residue T186 of SEQ ID NO: 1 may be a T186D substitution. In another example, the amino acid substitution at residue T186 of SEQ ID NO: 1 may be a T1 86G substitution.

In some embodiments, the LgtA polypeptide includes an amino acid substitution at residue M187 of SEQ ID NO: 1. For example, the amino acid substitution at residue M187 of SEQ ID NO: 1 may be an M1 87P substitution.

In some embodiments, the LgtA polypeptide includes an amino acid substitution at residue W206 of SEQ ID NO: 1. The amino acid substitution at residue W206 of SEQ ID NO: 1 may substitute W206 with an amino acid including a polar, uncharged side chain at physiological pH. For example, the amino acid substitution at residue W206 of SEQ ID NO: 1 may be a W206N substitution.

In some embodiments, the LgtA polypeptide includes an amino acid substitution at residue A207 of SEQ ID NO: 1. In some embodiments, the amino acid substitution at residue A207 of SEQ ID NO: 1 substitutes A207 with an amino acid including a hydrophobic, uncharged side chain at physiological pH. For example, the amino acid substitution at residue A207 of SEQ ID NO: 1 may be an A207V substitution.

In some embodiments, the LgtA polypeptide includes an amino acid substitution at residue Q211 of SEQ ID NO: 1. The amino acid substitution at residue Q211 of SEQ ID NO: 1 may substitute Q211 with an amino acid including a hydrophobic, uncharged side chain at physiological pH. For example, the amino acid substitution at residue Q211 of SEQ ID NO: 1 may be a Q211 V substitution, a Q211I substitution, or a Q211L substitution. In some embodiments, the amino acid substitution at residue Q211 of SEQ ID NO: 1 is a Q211C substitution.

In some embodiments, the LgtA polypeptide includes an amino acid substitution at residue W213 of SEQ ID NO: 1. In some embodiments, the amino acid substitution at residue W213 of SEQ ID NO: 1 substitutes W213 with an amino acid including a polar, uncharged side chain at physiological pH. For example, the amino acid substitution at residue W213 of SEQ ID NO: 1 is a W213S substitution or a W213N substitution.

In some embodiments, the LgtA polypeptide includes an amino acid substitution at residue L229 of SEQ ID NO: 1. In some embodiments, the amino acid substitution at residue L229 of SEQ ID NO: 1 substitutes L229 with an amino acid including a hydrophobic, uncharged side chain at physiological pH. For example, the amino acid substitution at residue L229 of SEQ ID NO: 1 may be an L229A substitution. In some embodiments, the amino acid substitution at residue L229 of SEQ ID NO: 1 is an L229P substitution.

In some embodiments, the LgtA polypeptide includes an amino acid substitution at residue V230 of SEQ ID NO: 1. The amino acid substitution at residue V230 of SEQ ID NO: 1 may substitute V230 with an amino acid including an anionic side chain at physiological pH. For example, the amino acid substitution at residue V230 of SEQ ID NO: 1 is a V230D substitution.

In some embodiments, the LgtA polypeptide includes an amino acid substitution at residue R233 of SEQ ID NO: 1. The amino acid substitution at residue R233 of SEQ ID NO: 1 may substitute R233 with an amino acid including a hydrophobic, uncharged side chain at physiological pH. For example, the amino acid substitution at residue R233 of SEQ ID NO: 1 may be an R233I substitution.

In some embodiments, the LgtA polypeptide includes an amino acid substitution at residue H235 of SEQ ID NO: 1. In some embodiments, the amino acid substitution at residue H235 of SEQ ID NO: 1 substitutes H235 with an amino acid including a cationic side chain at physiological pH. For example, the amino acid substitution at residue H235 of SEQ ID NO: 1 may be an H235R substitution.

In some embodiments, the LgtA polypeptide includes an amino acid substitution at residue S240 of SEQ ID NO: 1. The amino acid substitution at residue S240 of SEQ ID NO: 1 may substitute S240 with an amino acid including a polar, uncharged side chain at physiological pH. For example, the amino acid substitution at residue S240 of SEQ ID NO: 1 may be an S240N substitution. Furthermore, the amino acid substitution at residue S240 of SEQ ID NO: 1 may be an S240Y substitution.

In some embodiments, the LgtA polypeptide includes an amino acid substitution at residue K242 of SEQ ID NO: 1. In some embodiments, the amino acid substitution at residue K242 of SEQ ID NO: 1 substitutes K242 with an amino acid including an anionic side chain at physiological pH. For example, the amino acid substitution at residue K242 of SEQ ID NO: 1 may be a K242D substitution.

In some embodiments, the LgtA polypeptide includes an amino acid substitution at residue Y243 of SEQ ID NO: 1. The amino acid substitution at residue Y243 of SEQ ID NO: 1 may substitute Y243 with an amino acid including a polar, uncharged side chain at physiological pH. For example, the amino acid substitution at residue Y243 of SEQ ID NO: 1 may be a Y243S substitution. Furthermore, the amino acid substitution at residue Y243 of SEQ ID NO: 1 may be a Y243A substitution or a Y243L substitution. In some embodiments, the amino acid substitution at residue Y243 of SEQ ID NO: 1 substitutes Y243 with an amino acid including a cationic side chain at physiological pH. For example, the amino acid substitution at residue Y243 of SEQ ID NO: 1 may be a Y243R substitution.

In some embodiments, the LgtA polypeptide includes an amino acid substitution at residue Q247 of SEQ ID NO: 1. For example, the amino acid substitution at residue Q247 of SEQ ID NO: 1 may be a Q247C substitution.

In some embodiments, the LgtA polypeptide includes an amino acid substitution at residue L288 of SEQ ID NO: 1. In some embodiments, the amino acid substitution at residue L288 of SEQ ID NO: 1 substitutes L288 with an amino acid including a polar, uncharged side chain at physiological pH. For example, the amino acid substitution at residue L288 of SEQ ID NO: 1 may be a L288S substitution.

In some embodiments, the LgtA polypeptide includes an amino acid substitution at residue I250 of SEQ ID NO: 1. In some embodiments, the amino acid substitution at residue I250 of SEQ ID NO: 1 substitutes I250 with an amino acid including a hydrophobic, uncharged side chain at physiological pH. For example, the amino acid substitution at residue I250 of SEQ ID NO: 1 may be an I250F substitution.

In some embodiments, the LgtA polypeptide includes an amino acid substitution at residue I254 of SEQ ID NO: 1. In some embodiments, the amino acid substitution at residue I254 of SEQ ID NO: 1 substitutes I254 with an amino acid including a hydrophobic, uncharged side chain at physiological pH. For example, the amino acid substitution at residue I254 of SEQ ID NO: 1 may be an 1254A substitution.

In some embodiments, the LgtA polypeptide includes an amino acid substitution at residue Q255 of SEQ ID NO: 1. The amino acid substitution at residue Q255 of SEQ ID NO: 1 may substitute Q255 with an amino acid including an anionic side chain at physiological pH. For example, the amino acid substitution at residue Q255 of SEQ ID NO: 1 may be a Q255D substitution.

In some embodiments, the LgtA polypeptide includes an amino acid substitution at residue A258 of SEQ ID NO: 1. In some embodiments, the amino acid substitution at residue A258 of SEQ ID NO: 1 substitutes A258 with an amino acid including an anionic side chain at physiological pH. For example, in some embodiments, the amino acid substitution at residue A258 of SEQ ID NO: 1 is an A258D substitution. Furthermore, in some embodiments, the amino acid substitution at residue A258 of SEQ ID NO: 1 is an A258R substitution.

In some embodiments, the LgtA polypeptide includes an amino acid substitution at residue E294 of SEQ ID NO: 1. In some embodiments, the amino acid substitution at residue E294 of SEQ ID NO: 1 substitutes E294 with an amino acid including a polar, uncharged side chain at physiological pH. In some embodiments, the amino acid substitution at residue E294 of SEQ ID NO: 1 is an E294N substitution.

In some embodiments, the one or more amino acid substitutions include a deletion of residues 301-348 of SEQ ID NO: 1.

Illustrative variant LgtA polypeptide sequences that may be used in conjunction with the compositions and methods described herein include, without limitation, SEQ ID NO: 2-13, as well as functional variants thereof.

In some embodiments, the LgtA polypeptide has an amino acid sequence that is from about 85% to about 99.7% identical (e.g., about 86%, 87%, 88%, 89%, 90%, 91%, 92%, 93%, 94%, 95%, 96%, 97%, 98%, 99%, or 99.5% identical) to the amino acid sequence of SEQ ID NO: 1. In some embodiments, the polypeptide has an amino acid sequence that is from about 90% to about 99.7% identical (e.g., about 91%, 92%, 93%, 94%, 95%, 96%, 97%, 98%, 99%, 99.5% identical) to the amino acid sequence of SEQ ID NO: 1. In some embodiments, the polypeptide has an amino acid sequence that is from about 95% to about 99.7% identical (e.g., about 96%, 97%, 98%, 99%, or 99.5% identical) to the amino acid sequence of SEQ ID NO: 1.

In some embodiments, the LgtA polypeptide has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NO: 2-13. In some embodiments, the LgtA polypeptide has an amino acid sequence that is at least 90% identical (e.g., at least 91%, 92%, 93%, 94%, 95%, 96%, 97%, 98%, or 99% identical) to the amino acid sequence of any one of SEQ ID NO: 2-13. In some embodiments, the LgtA polypeptide has an amino acid sequence that is at least 95% identical (e.g., at least 95%, 96%, 97%, 98%, or 99% identical) to the amino acid sequence of any one of SEQ ID NO: 2-13. In some embodiments, the LgtA polypeptide has an amino acid sequence of any one of SEQ ID NO: 2-13.

### β-1,4-Galactosyltransferase Polypeptides

In some embodiments, the host cells of the disclosure express a LgtB polypeptide. In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 15. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 15. In some embodiments, the LgtB has an amino acid sequence that is at least 95% identical (e.g., at least 96%, 97%, 98%, or 99% identical) to the amino acid sequence of SEQ ID NO: 15. In some embodiments, the LgtB has the amino acid sequence of SEQ ID NO: 15.

In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 16. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 16. In some embodiments, the LgtB has an amino acid sequence that is at least 95% identical (e.g., at least 96%, 97%, 98%, or 99% identical) to the amino acid sequence of SEQ ID NO: 16. In some embodiments, the LgtB has the amino acid sequence of SEQ ID NO: 16.

### Lactose Importer

In some embodiments, the host cells of the disclosure express a protein that transports lactose into the host cell. In some embodiments, the protein is a lactose permease. In some embodiments, the lactose permease has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 17. In some embodiments, the lactose permease has an amino acid sequence that is at least 90% identical (e.g., at least 91%, 92%, 93%, 94%, 95%, 96%, 97%, 98%, or 99% identical) to the amino acid sequence of SEQ ID NO: 17. In some embodiments, the lactose permease has an amino acid sequence that is at least 95% identical (e.g., at least 96%, 97%, 98%, or 99% identical) to the amino acid sequence of SEQ ID NO: 17. In some embodiments, the lactose permease has the amino acid sequence of SEQ ID NO: 17.

### GDP-Mannose 4,6-Dehydratase

In some embodiments, the host cells of the disclosure express a GDP-mannose 4,6-dehydratase. In some embodiments, the GDP-mannose 4,6-dehydratase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 18. In some embodiments, the GDP-mannose 4,6-dehydratase has an amino acid sequence that is at least 90% identical (e.g., at least 91%, 92%, 93%, 94%, 95%, 96%, 97%, 98%, or 99% identical) to the amino acid sequence of SEQ ID NO: 18. In some embodiments, the GDP-mannose 4,6-dehydratase has an amino acid sequence that is at least 95% identical (e.g., at least 96%, 97%, 98%, or 99% identical) to the amino acid sequence of SEQ ID NO: 19. In some embodiments, the GDP-mannose 4,6-dehydratase has the amino acid sequence of SEQ ID NO: 18.

### GDP-L-Fucose Synthase

In some embodiments, the host cells of the disclosure express a GDP-L-fucose synthase. In some embodiments, the GDP-L-fucose synthase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 19. In some embodiments, the GDP-L-fucose synthase has an amino acid sequence that is at least 90% identical (e.g., at least 91%, 92%, 93%, 94%, 95%, 96%, 97%, 98%, or 99% identical) to the amino acid sequence of SEQ ID NO: 19. In some embodiments, the GDP-L-fucose synthase has an amino acid sequence that is at least 95% identical (e.g., at least 96%, 97%, 98%, or 99% identical) to the amino acid sequence of SEQ ID NO: 19. In some embodiments, the GDP-L-fucose synthase has the amino acid sequence of SEQ ID NO: 19.

α-1,2-fucosyltransferase

In some embodiments, the host cells of the disclosure express an α-1,2-fucosyltransferase polypeptide. In some embodiments, the α-1,2-fucosyltransferase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 20. In some embodiments, the α-1,2-fucosyltransferase has an amino acid sequence that is at least 90% identical (e.g., at least 91%, 92%, 93%, 94%, 95%, 96%, 97%, 98%, or 99% identical) to the amino acid sequence of SEQ ID NO: 20. In some embodiments, the α-1,2-fucosyltransferase has an amino acid sequence that is at least 95% identical (e.g., at least 96%, 97%, 98%, or 99% identical) to the amino acid sequence of SEQ ID NO: 20. In some embodiments, the α-1,2-fucosyltransferase has the amino acid sequence of SEQ ID NO: 20.

### Host Cells Genetically Modified to Produce HMOs

Provided herein are genetically modified host cells (e.g., yeast cells) capable of producing one or more HMOs, such as one or more of LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II, among others.

In some embodiments, a host cell (e.g., yeast cell) of the disclosure is genetically modified so as to express a LgtA polypeptide having an amino acid sequence of any one of SEQ ID NO: 2-13, or a biologically active variant that shares substantial identity with the amino acid sequence of any one of SEQ ID NO: 2-13. In some embodiments, the variant has at least 70%, 75%, 80%, 85%, 86%, 87%, 88%, 89%, 90%, 91%, 92%, 93%, 94%, 95%, 96%, 97%, 98%, 99%, or 100% identity to the amino acid sequence of any one of SEQ ID NO: 2-13.

LgtA activity can be assessed using any number of assays, including assays that evaluate the overall production of at least one HMO (e.g., LNnT, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, LST a, LST b, LST c, DS-LNT, F-LST a, F-LST b, FS-LNH, FS-LNnH I, or FDS-LNH II) by a host cell (e.g., yeast cell) strain. For example, production yields may be calculated by quantifying sugar input into fermentation tanks and measuring residual levels of input sugars through ion exchange chromatography. Additional methods that may be used to assess HMO production include mass spectrometry.

In some embodiments, a variant LgtA polypeptide increases HMO production, for example, by at least 10%, at least 20%, at least 30%, at least 40%, at least 50%, or greater, when expressed in a host cell (e.g., a yeast strain described herein) as compared to a counterpart host cell of the same strain that expresses a wild-type LgtA polypeptide.

In some embodiments, a variant LgtA polypeptide increases the purity of the HMO produced, e.g., by at least 10%, at least 20%, at least 30%, at least 40%, at least 50%, or greater, when expressed in a host cell compared to a counterpart host cell of the same strain that expresses a wild-type LgtA polypeptide.

In some embodiments, a variant LgtA polypeptide decreases undesired byproduct (e.g., para-LNnH) production by at least 10%, at least 20%, at least 30%, at least 40%, at least 50%, or greater, when expressed in a host cell compared to a counterpart host cell of the same strain that expresses a wild-type LgtA polypeptide.

In addition to expressing a LgtA polypeptide, host cells (e.g., yeast cells) of the disclosure may express a LgtB polypeptide. For example, host cells (e.g., yeast cells) of the disclosure may express a LgtB polypeptide having the amino acid sequence of SEQ ID NO: 15 or SEQ ID NO: 16, or a variant LgtB polypeptide having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 15 or SEQ ID NO: 16.

Host Cells Capable of Producing Exemplary HMOs and their Precursors

In some embodiments, the host cells of the disclosure are capable of producing one or more HMOs (e.g., LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II) and their precursors. The sections that follow describe host cells that are capable of producing exemplary HMOs, as well as the biosynthetic pathways that are involved in the production of each exemplary HMO.

In some embodiments, the host cells (e.g., yeast cells) of the disclosure are capable of producing the UDP-glucose HMO precursor. The activated sugar UDP-glucose is composed of a pyrophosphate group, the pentose sugar ribose, glucose, and the nucleobase uracil. UDP-glucose is natively produced by yeast cells, and its production levels can be increased with overexpression of, for example, phosphoglucomutase-2 (PGM2) or UTP glucose-1-phosphate uridylyltransferase (UGP1).

In some embodiments, the host cells (e.g., yeast cells) of the disclosure are capable of producing the UDP-galactose HMO precursor. The activated sugar UDP-galactose is composed of a pyrophosphate group, the pentose sugar ribose, galactose, and the nucleobase uracil. UDP-galactose is natively produced by yeast cells, and its production levels can be increased with overexpression of, for example, UDP-glucose-4-epimerase (GAL10).

In some embodiments, the host cells (e.g., yeast cells) of the disclosure are capable of producing the UDP-N-acetylglucosamine HMO precursor. The activated sugar UDP-N-acetylglucosamine consists of a pyrophosphate group, the pentose sugar ribose, N-acetylglucosamine, and the nucleobase uracil. UDP-N-acetylglucosamine is natively produced by yeast cells, and its production levels can be increased with expression of, for example, UDP-N-acetylglucosamine-diphosphorylase, or overexpression of, for example, glucosamine 6-phosphate N-acetyltransferase (GNA1) or phosphoacetylglucosamine mutase (PCM1).

In some embodiments, the host cells (e.g., yeast cells) of the disclosure are capable of producing the GDP-fucose HMO precursor. The activated sugar GDP-fucose consists of a pyrophosphate group, the pentose sugar ribose, fucose, and the nucleobase guanine. GDP-fucose is not natively produced by yeast cells, and its production can be enabled with the introduction of, for example, GDP-mannose 4,6-dehydratase, e.g., from Escherichia coli, and GDP-L-fucose synthase, e.g., from Arabidopsis thaliana.

In some embodiments, the host cells (e.g., yeast cells) of the disclosure are capable of producing the CMP-sialic acid HMO precursor. The activated sugar CMP-sialic acid consists of a pyrophosphate group, the pentose sugar ribose, sialic acid, and the nucleobase cytosine. CMP-sialic acid is not natively produced by yeast cells, and its production can be enabled with the introduction of, for example, CMP-Neu5Ac synthetase, e.g., from Campylobacter jejuni, sialic acid synthase, e.g., from C. jejuni, and UDP-N-acetylglucosamine 2-epimerase, e.g., from C. jejuni.

In some embodiments, the host cells (e.g., yeast cells) of the disclosure are capable of producing 2′-fucosyllactose. In addition to one or more heterologous nucleic acids encoding one or more of the aforementioned enzymes, the host cell may further include one or more heterologous nucleic acids encoding one or more of GDP-mannose 4,6-dehydratase, e.g., from Escherichia coli, GDP-L-fucose synthase, e.g., from Arabidopsis thaliana, a-1,2-fucosyltransferase, e.g., from Helicobacter pylori, and a fucosidase, e.g., an α-1,3-fucosidase. In some embodiments, the fucosyltransferase is from Candidata moranbacterium or Pseudoalteromonas haloplanktis.

In some embodiments, the host cells (e.g., yeast cells) of the disclosure may include a heterologous nucleic acid encoding an enzyme that can catalyze the conversion of GDP-mannose to GDP-4-dehydro-6-deoxy-D-mannose, e.g., a GDP-mannose 4,6-dehydratase. In some embodiments, the GDP-mannose 4,6-dehydratase is from Escherichia coli. Other suitable GDP-mannose 4,6-dehydratase sources include, for example and without limitation, Caenorhabditis elegans, Homo sapiens, Arabidopsis thaliana, Dictyostelium discoideum, Mus musculus, Drosophila melanogaster, Sinorhizobium fredii HH103, Sinorhizobium fredii NGR234, Planctomycetes bacterium RBG_13_63_9, Silicibacter sp. TrichCH4B, Pandoraea vervacti, Bradyrhizobium sp. YR681, Epulopiscium sp. SCG-B11 WGA-EpuloA1, Caenorhabditis briggsae, Candidatus Curtissbacteria bacterium RIFCSPLOWO2_12_FULL_38_9, Pseudomonas sp. EpS/L25, Clostridium sp. KLE 1755, Nitrospira sp. SG-bin2, Cricetulus griseus, Arthrobacter siccitolerans, and Paraburkholderia piptadeniae. In some embodiments, the GDP-mannose dehydratase is from Caenorhabditis briggsae or Escherichia coli.

In some embodiments, the host cells (e.g., yeast cells) of the disclosure may include a heterologous nucleic acid encoding an enzyme that can catalyze the conversion of GDP-4-dehydro-6-deoxy-D-mannose to GDP-L-fucose, e.g., a GDP-L-fucose synthase. In some embodiments, the GDP-L-fucose synthase is from Arabidopsis thaliana. Other suitable GDP-L-fucose synthase sources include, for example and without limitation, Mus musculus, Escherichia coli K-12, Homo sapiens, Marinobacter salarius, Sinorhizobium fredii NGR234, Oryza sativa Japonica Group, Micavibrio aeruginosavorus ARL-13, Citrobacter sp. 86, Pongo abelii, Caenorhabditis elegans, Candidatus Staskawiczbacteria bacterium RIFCSPHIGHO2_01_FULL_41_41, Drosophila melanogaster, Azorhizobium caulinodans ORS 571, Candidatus Nitrospira nitrificans, Mycobacterium elephantis, Elusimicrobia bacterium RBG_16_66_12, Vibrio sp. JCM 19231, Planktothrix serta PCC 8927, Thermodesulfovibrio sp. RBG_19FT_COMBO_42_12, Anaerovibrio sp. JC8, Dictyostelium discoideum, and Cricetulus griseus.

In some embodiments, the host cells (e.g., yeast cells) of the disclosure may include a heterologous nucleic acid encoding an enzyme that can catalyze the conversion of GDP-L-fucose and lactose to 2′-fucosyllactose, e.g., an α-1,2-fucosyltransferase. In some embodiments, the α-1,2-fucosyltransferase is from Helicobacter pylori. In some embodiments, the fucosyltransferase is from Candidata moranbacterium or Pseudoalteromonas haloplanktis ANT/505. Other suitable α-1,2-fucosyltransferase sources include, for example and without limitation, Escherichia coli, Sus scrofa, Homo sapiens, Chlorocebus sabaeus, Pan troglodytes, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, and Helicobacter hepaticus ATCC 51449. In some embodiments, the source of the α-1,2-fucosyltransferase is Pseudoalteromonas haloplanktis ANT/505, Candidatus moranbacteria bacterium, Acetobacter sp. CAG:267, Bacteroides vulgatus, Sulfurovum lithotrophicum, Thermosynechococcus elongatus BP-1, Geobacter uraniireducens Rf4, Bacteroides fragilis str. S23L17, Chromobacterium vaccinii, Herbaspirillum sp. YR522, or Helicobacter bilis ATCC 43879.

In some embodiments, the host cells (e.g., yeast cells) of the disclosure may include a heterologous nucleic acid encoding an enzyme that can catalyze the conversion of difucosyllactose to 2′-fucosyllactose and fucose, e.g., an a1-3,4-fucosidase. Suitable a1-3,4-fucosidase sources include, for example and without limitation, Bacteroides thetaiotaomicron, Bifidobacterium bifidum, Bifidobacterium longum, Bifidobacterium longum subsp. infantis, Clostridium perfringens, Lactobacillus casei, Paenibacillus thiaminolyticus, Pseudomonas putida, Thermotoga maritima, Arabidopsis thaliana, and Rattus norvegicus.

In some embodiments, the host cells (e.g., yeast cells) of the disclosure are capable of producing 3-fucosyllactose. In addition to one or more heterologous nucleic acids encoding one or more of the aforementioned enzymes, the host cell may further include one or more heterologous nucleic acids encoding one or more of GDP-mannose 4,6-dehydratase, e.g., from Escherichia coli, GDP-L-fucose synthase, e.g., from Arabidopsis thaliana, a-1,3-fucosyltransferase, e.g., from Helicobacter pylori, and a fucosidase, e.g., an α-1,2-fucosidase.

In some embodiments, the host cells (e.g., yeast cells) of the disclosure may include a heterologous nucleic acid encoding an enzyme that can catalyze the conversion of GDP-L-fucose and lactose to 3-fucosyllactose, e.g., an α-1,3-fucosyltransferase. In some embodiments, the α-1,3-fucosyltransferase is from Helicobacter pylori. Other suitable α-1,3-fucosyltransferase sources include, for example and without limitation, Homo sapiens, Escherichia coli, Sus scrofa, Chlorocebus sabaeus, Pan troglodytes, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, Helicobacter hepaticus ATCC 51449, Akkermansia muciniphila, Bacteroides fragilis, and Zea mays.

In some embodiments, the host cells (e.g., yeast cells) of the disclosure are capable of producing lacto-N-tetraose. In addition to one or more heterologous nucleic acids encoding one or more of the aforementioned enzymes, the host cell may further include one or more heterologous nucleic acids encoding one or more of β-1,3-N-acetylglucosaminyltransferase, e.g., from Neisseria meningitidis, p-1,3-galactosyltransferase, e.g., from Escherichia coli, and UDP-N-acetylglucosamine-diphosphorylase, e.g., from E. coli.

In some embodiments, the host cells (e.g., yeast cells) of the disclosure may include a heterologous nucleic acid encoding an enzyme that can catalyze the conversion of UDP-N-acetyl-alpha-D-glucosamine and lactose to lacto-N-triose II and UDP, e.g., a β-1,3-N-acetylglucosaminyltransferase. In some embodiments, the p3-1,3-N-acetylglucosaminyltransferase is from Neisseria meningitidis. Other suitable β-1,3-N-acetylglucosaminyltransferase sources include, for example and without limitation, Arabidopsis thaliana, Streptococcus dysgalactiae subsp. equisimilis, Escherichia coli, e.g., Escherichia coli K-12, Pseudomonas aeruginosa PAO1, Homo sapiens, Mus musculus, Mycobacterium smegmatis str. MC2 1 55, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Escherichia coli O157:H7, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula x Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus subsp. jinggangensis 5008, Drosophila melanogaster, Phytophthora infestans, Staphylococcus aureus subsp. aureus Mu50, Penicillium chrysogenum, and Tribolium castaneum.

In some embodiments, the host cells (e.g., yeast cells) of the disclosure may include a heterologous nucleic acid encoding an enzyme that can catalyze the conversion of UDP-galactose and lacto-N-triose II to lacto-N-tetraose and UDP, e.g., a β-1,3-galactosyltransferase. In some embodiments, the β-1,3-galactosyltransferase is from Escherichia coli. Other suitable β-1,3-galactosyltransferase sources include, for example and without limitation, Arabidopsis thaliana, Streptococcus dysgalactiae subsp. equisimilis, Pseudomonas aeruginosa PAO1, Homo sapiens, Mus musculus, Mycobacterium smegmatis str. MC2 1 55, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Neisseria meningitidis, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula x Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus subsp. jinggangensis 5008, Drosophila melanogaster, Phytophthora infestans, Staphylococcus aureus subsp. aureus Mu50, Penicillium chrysogenum, and Tribolium castaneum.

In some embodiments, the host cells (e.g., yeast cells) of the disclosure may include a heterologous nucleic acid encoding an enzyme that can catalyze the conversion of N-acetyl-α-D-glucosamine 1-phosphate to UDP-N-acetyl-α-D-glucosamine, e.g., a UDP-N-acetylglucosamine-diphosphorylase. In some embodiments, the UDP-N-acetylglucosamine-diphosphorylase is from Escherichia coli.

In some embodiments, the host cells (e.g., yeast cells) of the disclosure are capable of producing lacto-N-neotetraose. In addition to one or more heterologous nucleic acids encoding one or more of the aforementioned enzymes, the host cell may further include one or more heterologous nucleic acids encoding one or more of β-1,3-N-acetylglucosaminyltransferase, e.g., from Neisseria meningitidis, p-1,4-galactosyltransferase, e.g., from N. meningitidis, and UDP-N-acetylglucosamine-diphosphorylase, e.g., from E. coli.

In some embodiments, the host cells (e.g., yeast cells) of the disclosure may include a heterologous nucleic acid encoding an enzyme that can catalyze the conversion of UDP-galactose and lacto-N-triose II to lacto N-neotetraose and UDP, e.g., a β-1,4-galactosyltransferase. In some embodiments, the β-1,4-galactosyltransferase is from Neisseria meningitidis. Other suitable β-1,4-galactosyltransferase sources include, for example and without limitation, Homo sapiens, Neisseria gonorrhoeae, Haemophilus influenzae, Acanthamoeba polyphaga mimivirus, Haemophilus influenzae Rd KW20, Haemophilus ducreyi 35000HP, Moraxella catarrhalis, [Haemophilus] ducreyi, Aeromonas salmonicida subsp. salmonicida A449, and Helicobacter pylon 26695.

In some embodiments, the host cells (e.g., yeast cells) of the disclosure are capable of producing 3′-sialyllactose. In addition to heterologous nucleic acids encoding one or more of the aforementioned enzymes, the host cells may further include heterologous nucleic acids encoding CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E. coli, and CMP-N-acetylneuraminate-β-galactosamide-α-2,3-sialyltransferase, e.g., from N. meningitides MC58.

In some embodiments, the host cells (e.g., yeast cells) of the disclosure may include a heterologous nucleic acid encoding an enzyme that can catalyze the conversion of UDP-N-acetyl-α-D-glucosamine to N-acetyl-mannosamine and UDP, e.g., a UDP-N-acetylglucosamine 2-epimerase. In some embodiments, the UDP-N-acetylglucosamine 2-epimerase is from Campylobacter jejuni. Other suitable UDP-N-acetylglucosamine 2-epimerase sources include, for example and without limitation, Homo sapiens, Rattus norvegicus, Mus musculus, Dictyostelium discoideum, Plesiomonas shigelloides, Bacillus subtilis subsp. subtilis str. 168, Bacteroides fragilis, Geobacillus kaustophilus HTA426, Synechococcus sp. CC9311, Sphingopyxis alaskensis RB2256, Synechococcus sp. RS9916, Moorella thermoacetica ATCC 39073, Psychrobacter sp. 1501(2011), Zunongwangia profunda SM-A87, Thiomicrospira crunogena XCL-2, Polaribacter sp. MED152, Vibrio campbellii ATCC BAA-1116, Thiomonas arsenitoxydans, Nitrobacter winogradskyi Nb-255, Raphidiopsis brookii D9, Thermoanaerobacter italicus Ab9, Roseobacter litoralis Och 149, Halothiobacillus neapolitanus c2, Halothiobacillus neapolitanus c2, Bacteroides vulgatus ATCC 8482, Zunongwangia profunda SM-A87, Moorella thermoacetica ATCC 39073, Paenibacillus polymyxa E681, Desulfatibacillum alkenivorans AK-01, Magnetospirillum magneticum AMB-1, Thermoanaerobacter italicus Ab9, Paenibacillus polymyxa E681, Prochlorococcus marinus str. MIT 9211, Subdoligranulum variabile DSM 15176, Kordia algicida OT-1, Bizionia argentinensis JUB59, Tannerella forsythia 92A2, Thiomonas arsenitoxydans, Synechococcus sp. BL1107, Escherichia coli, Vibrio campbellii ATCC BAA-1116, Rhodopseudomonas palustris HaA2, Roseobacter litoralis Och 149, Synechococcus sp. CC9311, Subdoligranulum variabile DSM 15176, Bizionia argentinensis JUB59, Selenomonas sp. oral taxon 149 str. 67H29BP, Bacteroides vulgatus ATCC 8482, Kordia algicida OT-1, Desulfatibacillum alkenivorans AK-01, Thermodesulfovibrio yellowstonii DSM 11347, Desulfovibrio aespoeensis Aspo-2, Synechococcus sp. BL1107, and Desulfovibrio aespoeensis Aspo-2.

In some embodiments, the host cells (e.g., yeast cells) of the disclosure may include a heterologous nucleic acid encoding an enzyme that can catalyze the conversion of N-acetyl-mannosamine and phosphoenolpyruvate to N-acetylneuraminate, e.g., a sialic acid synthase. In some embodiments, the sialic acid synthase is from Campylobacter jejuni. Other suitable sialic acid synthase sources include, for example and without limitation, Homo sapiens, groundwater metagenome, Prochlorococcus marinus str. MIT 9211, Rhodospirillum centenum SW, Rhodobacter capsulatus SB 1003, Aminomonas paucivorans DSM 12260, Ictalurus punctatus, Octadecabacter antarcticus 307, Octadecabacter arcticus 238, Butyrivibrio proteoclasticus B316, Neisseria meningitidis serogroup B., Idiomarina loihiensis L2TR, Butyrivibrio proteoclasticus B316, and Campylobacter jejuni.

In some embodiments, the host cells (e.g., yeast cells) of the disclosure may include a heterologous nucleic acid encoding an enzyme that can catalyze the conversion of N-acetylneuraminate and CTP to CMP-N-acetylneuraminate, e.g., a CMP-Neu5Ac synthetase. In some embodiments, the CMP-Neu5Ac synthetase is from Campylobacter jejuni. Other suitable CMP-Neu5Ac synthetase sources include, for example and without limitation, Neisseria meningitidis, Streptococcus agalactiae NEM316, Homo sapiens, Mus musculus, Bacteroides thetaiotaomicron, Pongo abelii, Danio rerio, Oncorhynchus mykiss, Bos taurus, Drosophila melanogaster, and Streptococcus suis BM407.

In some embodiments, the host cells (e.g., yeast cells) of the disclosure may include a heterologous nucleic acid encoding an enzyme that can catalyze the conversion of CMP-N-acetylneuraminate and lactose to 3′-siallyllactose and CMP, e.g., a CMP-N-acetylneuraminate-p-galactosamide-α-2,3-sialyltransferase. In some embodiments, the CMP-N-acetylneuraminate-p-galactosamide-α-2,3-sialyltransferase is from N. meningitides MC58. Other suitable CMP-N-acetylneuraminate-β-galactosamide-α-2,3-sialyltransferase sources include, for example and without limitation, Homo sapiens, Neisseria meningitidis alpha14, Pasteurella multocida subsp. multocida str. Pm70, Pasteurella multocida, and Rattus norvegicus.

In some embodiments, the host cells (e.g., yeast cells) of the disclosure are capable of producing 6′-sialyllactose. In addition to one or more heterologous nucleic acids encoding one or more of the aforementioned enzymes, the host cell may further include one or more heterologous nucleic acids encoding one or more of CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E. coli, and β-galactoside α-2,6-sialyltransferase, e.g., from Photobacterium sp. JT-ISH-224.

In some embodiments, the host cells (e.g., yeast cells) of the disclosure may include a heterologous nucleic acid encoding an enzyme that can catalyze the conversion of CMP-N-acetylneuraminate and lactose to 3′-sialyllactose and CMP, e.g., a β-galactoside-α-2,6-sialyltransferase. In some embodiments, the β-galactoside-α-2,6-sialyltransferase is from Photobacterium sp. JT-ISH-224. Other suitable β-galactoside-α-2,6-sialyltransferase sources include, for example and without limitation, Homo sapiens, Photobacterium damselae, Photobacterium leiognathi, and Photobacterium phosphoreum ANT-2200.

### Exemplary Host Cell Strains

In some embodiments, the host cell is a yeast cell, such as Saccharomyces cerevisiae. Saccharomyces cerevisiae strains suitable for genetic modification and cultivation to produce HMOs as disclosed herein include, but are not limited to, Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, CEN.PK, CEN.PK2, and AL-1. In some embodiments, the host cell is a strain of Saccharomyces cerevisiae selected from the group consisting of PE-2, CAT-1, VR-1, BG-1, CR-1, and SA-1. In certain aspects, the strain of Saccharomyces cerevisiae is PE-2. In certain embodiments, the strain of Saccharomyces cerevisiae is CAT-1. In some aspects, the strain of Saccharomyces cerevisiae is BG-1.

In some embodiments, the host cell is Saccharomyces cerevisiae, and in addition to heterologous nucleic acids encoding one or more of the aforementioned enzymes, the host cell may further include a heterologous nucleic acid encoding a lactose transporter. In some embodiments, the lactose transporter is a lactose permease, e.g., LAC12 from Kluyveromyces lactis (SEQ ID NO: 14). In some embodiments, the lactose permease is from Neurospora crassa, e.g., Cdt2. In some embodiments, the lactose permease is from Neofusicoccum parvum, e.g., Neofusicoccum parvum UCRNP2 (1287680). Other suitable lactose permease sources include, for example and without limitation, Scheffersomyces stipitis, Aspergillus lentulus, Emericella nidulans, Dacryopinax primogenitus, Microdochium bolleyi, Beauveria bassiana, Metarhizium robertsii, Phialocephala, Botryosphaeria parva, Moniliophthora roreri, Cordyceps fumosorosea, Diplodia seriata, Hypocrea jecorina, and Kluyveromyces marxianus.

In some embodiments, in addition to heterologous nucleic acids encoding one or more of the aforementioned enzymes, the host cell may further include a LgtB polypeptide. In some embodiments, the LgtB polypeptide is from Pasteurella multocida (SEQ ID NO: 15).

In some embodiments, in addition to heterologous nucleic acids encoding one or more of the aforementioned enzymes, the host cell may further include a LgtB polypeptide. In some embodiments, the LgtB polypeptide is from Neisseria gonorrhoeae (SEQ ID NO: 16).

In some embodiments, the host cell is Kluyveromyces marxianus. Kluyveromyces marxianus can provide several advantages for industrial production, including high temperature tolerance, acid tolerance, native uptake of lactose, and rapid growth rate. Beneficially, this yeast has sufficient genetic similarity to Saccharomyces cerevisiae such that similar or identical promoters and codon optimized genes can be used among the two yeast species. Furthermore, because Kluyveromyces marxianus has a native lactose permease, it is not necessary to introduce a heterologous nucleic acid to introduce this functionality. In some embodiments, at least a portion of the β-galactosidase gene (LAC4) required for metabolizing lactose is deleted in the genetically modified yeast. Thus, the modified Kluyveromyces marxianus strain is capable of importing lactose without consuming it. In some embodiments, the expression of the β-galactosidase gene in the genetically modified yeast is decreased relative to the expression in wild-type Kluyveromyces marxianus. Thus, the modified Kluyveromyces marxianus strain has reduced consumption of imported lactose.

### Gene Expression Regulatory Elements

In some embodiments, the host cells (e.g., yeast cells) of the disclosure may include a promoter that regulates the expression and/or stability of at least one of the heterologous nucleic acids described herein. In certain aspects, the promoter negatively regulates the expression and/or stability of the at least one heterologous nucleic acid. The promoter can be responsive to a small molecule that may be present in a culture medium containing the host cell. In some embodiments, the small molecule is maltose or an analog or derivative thereof. In some embodiments, the small molecule is lysine or an analog or derivative thereof. Maltose and lysine can be attractive selections for the small molecule as they are relatively inexpensive, non-toxic, and stable.

In some embodiments, the promoter that regulates expression of a heterologous nucleic acid described herein is a relatively weak promoter, or an inducible promoter. Illustrative promoters include, for example, lower-strength GAL pathway promoters, such as GAL10, GAL2, and GAL3 promoters. Additional illustrative promoters for use in conjunction with the heterologous nucleic acids of the disclosure include constitutive promoters from S. cerevisiae, such as the promoter from the native TDH3 gene. In some embodiments, a lower strength promoter provides a decrease in expression of at least 25%, or at least 30%, 40%, or 50%, or more, when compared to a GAL1 promoter.

Expression of a heterologous nucleic acid molecule described herein may be accomplished by introducing the heterologous nucleic acid into the host cells under the control of regulatory elements that permit expression in the host cell. In some embodiments, the heterologous nucleic acid is an extrachromosomal plasmid. In some embodiments, the heterologous nucleic acid is a chromosomal integration vector that can integrate the nucleotide sequence of interest into the chromosome of the host cell.

Introduction of Heterologous Nucleic Acids into a Host Cell

In some embodiments, a heterologous nucleic acid of the disclosure is introduced into a host cell (e.g., yeast cell) by way of a gap repair molecular biology technique. In these methods, if the host cell has non-homologous end joining (NHEJ) activity, as is the case for Kluyveromyces marxianus, then the NHEJ activity in the host cell can be first disrupted in any of a number of ways. Further details related to genetic modification of host cells (e.g., yeast cells) through gap repair can be found in U.S. Pat. No. 9,476,065, the disclosure of which is incorporated herein by reference in its entirety.

In some embodiments, a heterologous nucleic acid of the disclosure is introduced into the host cell by way of one or more site-specific nucleases capable of causing breaks at designated regions within selected nucleic acid target sites. Examples of such nucleases include, but are not limited to, endonucleases, site-specific recombinases, transposases, topoisomerases, zinc finger nucleases, TAL-effector DNA binding domain-nuclease fusion proteins (TALENs), CRISPR/Cas-associated RNA-guided endonucleases, and meganucleases. Further details related to genetic modification of host cells through site specific nuclease activity can be found in U.S. Pat. No. 9,476,065, the disclosure of which is incorporated herein by reference in its entirety.

### Nucleic Acid and Amino Acid Sequence Optimization

Described herein are specific genes and proteins useful in the methods, compositions, and organisms of the disclosure; however, it will be recognized that absolute identity to such genes is not necessary. For example, changes in a particular gene or polynucleotide including a sequence encoding a polypeptide or enzyme can be performed and screened for activity. Typically, such changes include conservative mutations and silent mutations. Such modified or mutated polynucleotides and polypeptides can be screened for expression of a functional enzyme using methods known in the art. Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given heterologous polypeptide of the disclosure. A native DNA sequence encoding the biosynthetic enzymes described above is referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties, e.g., charge or hydrophobicity. In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol. Biol. 25: 365-89).

Furthermore, any of the genes encoding an enzyme described herein (or any of the regulatory elements that control or modulate expression thereof) can be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., Salmonella spp., or X. dendrorhous.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art can be suitable to identify analogous genes and analogous enzymes. Techniques include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, e.g., as described herein or in Kiritani, K., Branched-Chain Amino Acids Methods Enzymology, 1970; then isolating the enzyme with said activity through purification; determining the protein sequence of the enzyme through techniques such as Edman degradation; design of PCR primers to the likely nucleic acid sequence; amplification of said DNA sequence through PCR; and cloning of said nucleic acid sequence. To identify homologous or similar genes and/or homologous or similar enzymes, suitable techniques also include comparison of data concerning a candidate gene or enzyme with databases such as BRENDA, KEGG, or MetaCYC. The candidate gene or enzyme can be identified within the above mentioned databases in accordance with the teachings herein.

### Methods of Producing HMOs

Also provided herein are methods of producing one or more HMOs (e.g., one or more of LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II). For example, provided herein are methods for producing LNnT. The methods may include, for example, providing a population of host cells (e.g., yeast cells) capable of producing one or more HMOs and subsequently modifying the host cells so as to be deficient in expression and/or activity of an oxidoreductase described herein (e.g., an aldose reductase described herein).

In some embodiments, the host cells of the disclosure are cultured under conditions suitable for the production of a desired HMO. The culturing can be performed in a suitable culture medium in a suitable container, such as a cell culture plate, a flask, or a fermentor. Any suitable fermentor may be used, including, but not limited to, a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric et al., in Ullmann's Encyclopedia of Industrial Chemistry, Sixth Edition, Volume 12, pages 398-473, Wiley-VCH Verlag GmbH & Co. KDaA, Weinheim, Germany. Further, the methods can be performed at any scale of fermentation known in the art to support industrial production of microbial products. Materials and methods for the maintenance and growth of cell cultures are well known to those skilled in the art of microbiology or fermentation science (see, for example, Bailey et al., Biochemical Engineering Fundamentals, second edition, McGraw Hill, New York, 1986). Consideration should be given to appropriate culture medium, pH, temperature, and requirements for aerobic, microaerobic, or anaerobic conditions, depending on the specific requirements of the host cell, the fermentation, and the process.

In some embodiments, the culturing is carried out for a period of time sufficient for the transformed population to undergo a plurality of doublings until a desired cell density is reached. In some embodiments, the culturing is carried out for a period of time sufficient for the host cell population to reach a cell density (OD600) of between 0.01 and 400 in the fermentation vessel or container in which the culturing is being carried out. The culturing can be carried out until the cell density is, for example, between 0.1 and 14, between 0.22 and 33, between 0.53 and 76, between 1.2 and 170, or between 2.8 and 400. In terms of upper limits, the culturing can be carried until the cell density is no more than 400, e.g., no more than 170, no more than 76, no more than 33, no more than 14, no more than 6.3, no more than 2.8, no more than 1.2, no more than 0.53, or no more than 0.23. In terms of lower limits, the culturing can be carried out until the cell density is greater than 0.1, e.g., greater than 0.23, greater than 0.53, greater than 1.2, greater than 2.8, greater than 6.3, greater than 14, greater than 33, greater than 76, or greater than 170. Higher cell densities, e.g., greater than 400, and lower cell densities, e.g., less than 0.1, are also contemplated.

In other embodiments, the culturing is carried for a period of time, for example, between 12 hours and 92 hours, e.g., between 12 hours and 60 hours, between 20 hours and 68 hours, between 28 hours and 76 hours, between 36 hours and 84 hours, or between 44 hours and 92 hours. In some embodiments, the culturing is carried out for a period of time, for example, between 5 days and 20 days, e.g., between 5 days and 14 days, between 6.5 days and 15.5 days, between 8 days and 17 days, between 9.5 days and 18.5 days, or between 11 days and 20 days. In terms of upper limits, the culturing can be carried out for less than 20 days, e.g., less than 18.5 days, less than 17 days, less than 15.5 days, less than 14 days, less than 12.5 day, less than 11 days, less than 9.5 days, less than 8 days, less than 6.5 days, less than 5 day, less than 92 hours, less than 84 hours, less than 76 hours, less than 68 hours, less than 60 hours, less than 52 hours, less than 44 hours, less than 36 hours, less than 28 hours, or less than 20 hours. In terms of lower limits, the culturing can be carries out for greater than 12 hours, e.g., greater than 20 hours, greater than 28 hours, greater than 36 hours, greater than 44 hours, greater than 52 hours, greater than 60 hours, greater than 68 hours, greater than 76 hours, greater than 84 hours, greater than 92 hours, greater than 5 days, greater than 6.5 days, greater than 8 days, greater than 9.5 days, greater than 11 days, greater than 12.5 days, greater than 14 days, greater than 15.5 days, greater than 17 days, or greater than 18.5 days. Longer culturing times, e.g., greater than 20 days, and shorter culturing times, e.g., less than 5 hours, are also contemplated.

In certain embodiments, the production of the one or more HMOs by the population of host cells is inducible by an inducing compound. Such host cells can be manipulated with ease in the absence of the inducing compound. The inducing compound is then added to induce the production of one or more HMOs by the host cells. In other embodiments, production of the one or more HMOs by the host cells is inducible by changing culture conditions, such as, for example, the growth temperature, media constituents, and the like.

In certain embodiments, an inducing agent is added during a production stage to activate a promoter or to relieve repression of a transcriptional regulator associated with a biosynthetic pathway to promote production of one or more HMOs. In certain embodiments, an inducing agent is added during a build stage to repress a promoter or to activate a transcriptional regulator associated with a biosynthetic pathway to repress the production of one or more HMOs, and an inducing agent is removed during the production stage to activate a promoter or to relieve repression of a transcriptional regulator to promote the production of one or more HMOs.

As discussed above, in some embodiments, the host cells may include a promoter that regulates the expression and/or stability of a heterologous nucleic acid described herein. Thus, in certain embodiments, the promoter can be used to control the timing of gene expression and/or stability of proteins.

In some embodiments, when fermentation of a host cell capable of producing a desired HMO is carried out in the presence of a small molecule, e.g., at least about 0.1% maltose or lysine, HMO production is substantially reduced or eliminated. When the small molecule is removed from the fermentation culture medium, HMO production is stimulated. Such a system enables the use of the presence or concentration of a selected small molecule in a fermentation medium as a switch for the production of a HMO. Controlling the timing of non-catabolic compound production so as to occur only when production is desired redirects the carbon flux during the non-production phase into cell maintenance and biomass. This more efficient use of carbon can greatly reduce the metabolic burden on the host cells, improve cell growth, increase the stability of the heterologous genes, reduce strain degeneration, and/or contribute to better overall health and viability of the cells.

In some embodiments, the fermentation method includes a two-step process that utilizes a small molecule as a switch to affect the “off” and “on” stages. In the first step, i.e., the “build” stage, wherein production of the compound is not desired, the host cells are grown in a growth or “build” medium including the small molecule in an amount sufficient to induce the expression of genes under the control of a responsive promoter, and the induced gene products act to negatively regulate production of the non-catabolic compound. In the second step, i.e., the “production” stage, the fermentation is carried out in a culture medium including a carbon source wherein the small molecule is absent or present in sufficiently low amounts such that the activity of a responsive promoter is reduced or inactive. As a result, the production of the desired non-catabolic compound by the host cells is stimulated.

In some embodiments, the culture medium is any culture medium in which a host cell (e.g., yeast cell) can subsist, i.e., maintain growth and viability. In some embodiments, the culture medium is an aqueous medium including assimilable carbon, nitrogen, and phosphate sources. Such a medium can also include appropriate salts, minerals, metals, and other nutrients. In some embodiments, the carbon source and each of the essential cell nutrients are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells, which convert the carbon source to a biomass.

In some embodiments, the method of producing one or more HMOs includes culturing host cells in separate build and production culture media. For example, the method can include culturing the host cells in a build stage, wherein the cells are cultured under non-producing conditions, e.g., non-inducing conditions, thereby producing an inoculum. The inoculum may then be transferred into a second fermentation medium under conditions suitable to induce production of one or more HMOs, e.g., inducing conditions. Steady state conditions may then be maintained in the second fermentation stage so as to produce a cell culture containing one or more desired HMOs.

In some embodiments, the culture medium includes sucrose and lactose. In some embodiments, the carbon sources in the culture medium consist essentially of sucrose and lactose. In some embodiments, the carbon sources in the culture medium consist of sucrose and lactose. In some embodiments, the mass ratio of the sucrose to the lactose is selected to influence, adjust, or control the relative production rates of HMO(s) produced by the yeast cells. Controlling the composition of the produced HMO(s) in this way can advantageously permit the increasing of desired products, the decreasing of undesired products, the targeting of a desired product ratio, and the simplification of downstream product separation processes.

The mass ratio of the sucrose to the lactose in the culture medium can be, for example, between 3 and 40, e.g., between 3 and 25.6, between 7.6 and 29.2, between 11.2 and 32.8, between 14.8 and 36.4, between 18.4 and 40, between 3 and 10, between 3 and 5, or between 3 and 4. In terms of upper limits, the mass ratio of the sucrose to the lactose can be less than 40, e.g., less than 36.4, less than 32.8, less than 29.2, less than 25.6, less than 22, less than 18.4, less than 14.8, less than 11.2, less than 7.6, or less than 5. In terms of lower limits, the mass ratio of the sucrose to the lactose can be greater than 3, e.g., greater than 7.6, greater than 11.2, greater than 14.8, greater than 18.4, greater than 22, greater than 25.6, greater than 29.2, greater than 32.8, or greater than 36.4. Higher ratios, e.g., greater than 40, and lower ratios, e.g., less than 3, are also contemplated.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources in the culture medium is greater than about 0.1 g/L, preferably greater than about 0.25 g/L, and more preferably greater than about 1.0 g/L. In some embodiments, the addition of a nitrogen source to the culture medium beyond a certain concentration is not advantageous for the growth of the yeast. As a result, the concentration of the nitrogen sources in the culture medium can be less than about 20 g/L, e.g., less than about 10 g/L or less than about 5 g/L. Further, in some instances it may be desirable to allow the culture medium to become depleted of the nitrogen sources during culturing.

The effective culture medium can contain other compounds, such as inorganic salts, vitamins, trace metals, or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, e.g., greater than about 2.0 g/L or greater than about 5.0 g/L. In some embodiments, the addition of phosphate to the culture medium beyond certain concentrations is not advantageous for the growth of the yeast. Accordingly, the concentration of phosphate in the culture medium can be less than about 20 g/L, e.g., less than about 15 g/L or less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, e.g., greater than about 1.0 g/L or greater than about 2.0 g/L. In some embodiments, the addition of magnesium to the culture medium beyond certain concentrations is not advantageous for the growth of the yeast. Accordingly, the concentration of magnesium in the culture medium can be less than about 10 g/L, e.g, less than about 5 g/L or less than about 3 g/L. Further, in some instances it may be desirable to allow the culture medium to become depleted of a magnesium source during culturing.

In some embodiments, the culture medium can also include a biologically acceptable chelating agent, such as the dihydrate of trisodium citrate. In such instance, the concentration of a chelating agent in the culture medium can be greater than about 0.2 g/L, e.g., greater than about 0.5 g/L or greater than about 1 g/L. In some embodiments, the addition of a chelating agent to the culture medium beyond certain concentrations is not advantageous for the growth of the yeast. Accordingly, the concentration of a chelating agent in the culture medium can be less than about 10 g/L, e.g., less than about 5 g/L or less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, e.g., within the range of from about 20 mg/L to about 1000 mg/L or in the range of from about 50 mg/L to about 500 mg/L.

The culture medium can also include sodium chloride. Typically, the concentration of sodium chloride in the culture medium is within the range of from about 0.1 g/L to about 5 g/L, e.g., within the range of from about 1 g/L to about 4 g/L or in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the volume of such a trace metal solution added to the culture medium is greater than about 1 mL/L, e.g., greater than about 5 mL/L, and more preferably greater than about 10 mL/L. In some embodiments, the addition of a trace metals to the culture medium beyond certain concentrations is not advantageous for the growth of the host cells (e.g., yeast cells).

Accordingly, the amount of such a trace metals solution added to the culture medium may desirably be less than about 100 mL/L, e.g., less than about 50 mL/L or less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, inositol, pyridoxine-HCl, thiamine-HCl, and combinations thereof. Such vitamins can be added to the culture medium as a stock solution that, for convenience, can be prepared separately from the rest of the culture medium. In some embodiments, the addition of vitamins to the culture medium beyond certain concentrations is not advantageous for the growth of the host cells (e.g., yeast cells).

The fermentation methods described herein can be performed in conventional culture modes, which include, but are not limited to, batch, fed-batch, cell recycle, continuous, and semi-continuous. In some embodiments, the fermentation is carried out in fed-batch mode. In such a case, some of the components of the medium are depleted during culture, e.g., during the production stage of the fermentation. In some embodiments, the culture may be supplemented with relatively high concentrations of such components at the outset, for example, of the production stage, so that growth and/or HMO production (e.g., HMO production) is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those of ordinary skill in the art, the rate of consumption of nutrient increases during culture as the cell density of the medium increases. Moreover, to avoid introduction of foreign microorganisms into the culture medium, addition can be performed using aseptic addition methods, as are known in the art. In addition, a small amount of anti-foaming agent may be added during the culture.

The temperature of the culture medium can be any temperature suitable for growth of the host cells (e.g., yeast cells). For example, prior to inoculation of the culture medium with an inoculum, the culture medium can be brought to and maintained at a temperature in the range of from about 20° C. to about 45° C., e.g., to a temperature in the range of from about 25° C. to about 40° C., such as from about 28° C. to about 32° C. For example, the culture medium can be brought to and maintained at a temperature of 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C.

The pH of the culture medium can be controlled by the addition of acid or base to the culture medium. In such cases, when ammonia is used to control pH, it also conveniently serves as a nitrogen source in the culture medium. In some embodiments, the pH is maintained at from about 3.0 to about 8.0, e.g., at from about 3.5 to about 7.0 or from about 4.0 to about 6.5.

In some embodiments, the host cells (e.g., yeast cells) produce LNnT. The concentration of produced LNnT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNnT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNnT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNnT can be 40 g/l or greater, e.g., 50 g/l, 60 g/l, 70 g/l, 80 g/l, 90 g/l, or greater. For example, in some embodiments, concentrations of produced LNnT in the culture medium can be 100 g/l or greater.

The yield of produced LNnT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNnT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNT. The concentration of produced LNT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNT can be 40 g/l or greater, e.g., 50 g/l, 60 g/l, 70 g/l, 80 g/l, 90 g/l, or greater. For example, in some embodiments, concentrations of produced LNT in the culture medium can be 100 g/l or greater.

The yield of produced LNT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 2′-FL. The concentration of produced 2′-FL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 2′-FL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 2′-FL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 2′-FL can be 40 g/l or greater, e.g., 50 g/l, 60 g/l, 70 g/l, 80 g/l, 90 g/l, or greater. For example, in some embodiments, concentrations of produced 2′-FL in the culture medium can be 100 g/l or greater.

The yield of produced 2′-FL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 2′-FL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 3-FL. The concentration of produced 3-FL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 3-FL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 3-FL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 3-FL can be 40 g/l or greater, e.g., 50 g/l, 60 g/l, 70 g/l, 80 g/l, 90 g/l, or greater. For example, in some embodiments, concentrations of produced 3-FL in the culture medium can be 100 g/l or greater.

The yield of produced 3-FL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 3-FL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 6′-SL. The concentration of produced 6′-SL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 6′-SL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 6′-SL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 6′-SL can be 40 g/l or greater, e.g., 50 g/l, 60 g/l, 70 g/l, 80 g/l, 90 g/l, or greater. For example, in some embodiments, concentrations of produced 6′-SL in the culture medium can be 100 g/l or greater.

The yield of produced 6′-SL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 6′-SL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP 1. The concentration of produced LNFP I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP I can be 40 g/l or greater, e.g., 50 g/l, 60 g/l, 70 g/l, 80 g/l, 90 g/l, or greater. For example, in some embodiments, concentrations of produced LNFP I in the culture medium can be 100 g/l or greater.

The yield of produced LNFP I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP II. The concentration of produced LNFP II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP II can be 40 g/l or greater, e.g., 50 g/l, 60 g/l, 70 g/l, 80 g/l, 90 g/l, or greater. For example, in some embodiments, concentrations of produced LNFP II in the culture medium can be 100 g/l or greater.

The yield of produced LNFP II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP III. The concentration of produced LNFP III in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP III in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP III concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP III can be 40 g/l or greater, e.g., 50 g/l, 60 g/l, 70 g/l, 80 g/l, 90 g/l, or greater. For example, in some embodiments, concentrations of produced LNFP III in the culture medium can be 100 g/l or greater.

The yield of produced LNFP III on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP III on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP V. The concentration of produced LNFP V in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP V in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP V concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP V can be 40 g/l or greater, e.g., 50 g/l, 60 g/l, 70 g/l, 80 g/l, 90 g/l, or greater. For example, in some embodiments, concentrations of produced LNFP V in the culture medium can be 100 g/l or greater.

The yield of produced LNFP V on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP V on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP VI. The concentration of produced LNFP VI in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP VI in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP VI concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP VI can be 40 g/l or greater, e.g., 50 g/l, 60 g/l, 70 g/l, 80 g/l, 90 g/l, or greater. For example, in some embodiments, concentrations of produced LNFP VI in the culture medium can be 100 g/l or greater.

The yield of produced LNFP VI on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP VI on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 3′-SL. The concentration of produced 3′-SL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 3′-SL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 3′-SL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 3′-SL can be 40 g/l or greater, e.g., 50 g/l, 60 g/l, 70 g/l, 80 g/l, 90 g/l, or greater. For example, in some embodiments, concentrations of produced 3′-SL in the culture medium can be 100 g/l or greater.

The yield of produced 3′-SL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 3′-SL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNDFH I. The concentration of produced LNDFH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNDFH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNDFH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNDFH I can be 40 g/l or greater, e.g., 50 g/l, 60 g/l, 70 g/l, 80 g/l, 90 g/l, or greater. For example, in some embodiments, concentrations of produced LNDFH I in the culture medium can be 100 g/l or greater.

The yield of produced LNDFH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNDFH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNDFH II. The concentration of produced LNDFH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNDFH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNDFH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNDFH II can be 40 g/l or greater, e.g., 50 g/l, 60 g/l, 70 g/l, 80 g/l, 90 g/l, or greater. For example, in some embodiments, concentrations of produced LNDFH II in the culture medium can be 100 g/l or greater.

The yield of produced LNDFH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNDFH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNH. The concentration of produced LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNH can be 40 g/l or greater, e.g., 50 g/l, 60 g/l, 70 g/l, 80 g/l, 90 g/l, or greater. For example, in some embodiments, concentrations of produced LNH in the culture medium can be 100 g/l or greater.

The yield of produced LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNnH. The concentration of produced LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNnH can be 40 g/l or greater, e.g., 50 g/l, 60 g/l, 70 g/l, 80 g/l, 90 g/l, or greater. For example, in some embodiments, concentrations of produced LNnH in the culture medium can be 100 g/l or greater.

The yield of produced LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LNH I. The concentration of produced F-LNH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LNH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LNH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LNH I can be 40 g/l or greater, e.g., 50 g/l, 60 g/l, 70 g/l, 80 g/l, 90 g/l, or greater. For example, in some embodiments, concentrations of produced F-LNH I in the culture medium can be 100 g/l or greater.

The yield of produced F-LNH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LNH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LNH II. The concentration of produced F-LNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LNH II can be 40 g/l or greater, e.g., 50 g/l, 60 g/l, 70 g/l, 80 g/l, 90 g/l, or greater. For example, in some embodiments, concentrations of produced F-LNH II in the culture medium can be 100 g/l or greater.

The yield of produced F-LNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFL. The concentration of produced DFL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFL can be 40 g/l or greater, e.g., 50 g/l, 60 g/l, 70 g/l, 80 g/l, 90 g/l, or greater. For example, in some embodiments, concentrations of produced DFL in the culture medium can be 100 g/l or greater.

The yield of produced DFL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNH I. The concentration of produced DFLNH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNH I can be 40 g/l or greater, e.g., 50 g/l, 60 g/l, 70 g/l, 80 g/l, 90 g/l, or greater. For example, in some embodiments, concentrations of produced DFLNH I in the culture medium can be 100 g/l or greater.

The yield of produced DFLNH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNH II. The concentration of produced DFLNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNH II can be 40 g/l or greater, e.g., 50 g/l, 60 g/l, 70 g/l, 80 g/l, 90 g/l, or greater. For example, in some embodiments, concentrations of produced DFLNH II in the culture medium can be 100 g/l or greater.

The yield of produced DFLNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNnH. The concentration of produced DFLNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNnH can be 40 g/l or greater, e.g., 50 g/l, 60 g/l, 70 g/l, 80 g/l, 90 g/l, or greater. For example, in some embodiments, concentrations of produced DFLNnH in the culture medium can be 100 g/l or greater.

The yield of produced DFLNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DF-para-LNH. The concentration of produced DF-para-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DF-para-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DF-para-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DF-para-LNH can be 40 g/l or greater, e.g., 50 g/l, 60 g/l, 70 g/l, 80 g/l, 90 g/l, or greater. For example, in some embodiments, concentrations of produced DF-para-LNH in the culture medium can be 100 g/l or greater.

The yield of produced DF-para-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DF-para-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DF-para-LNnH. The concentration of produced DF-para-LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DF-para-LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DF-para-LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DF-para-LNnH can be 40 g/l or greater, e.g., 50 g/l, 60 g/l, 70 g/l, 80 g/l, 90 g/l, or greater. For example, in some embodiments, concentrations of produced DF-para-LNnH in the culture medium can be 100 g/l or greater.

The yield of produced DF-para-LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DF-para-LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce TF-LNH. The concentration of produced TF-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced TF-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the TF-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced TF-LNH can be 40 g/l or greater, e.g., 50 g/l, 60 g/l, 70 g/l, 80 g/l, 90 g/l, or greater. For example, in some embodiments, concentrations of produced TF-LNH in the culture medium can be 100 g/l or greater.

The yield of produced TF-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of TF-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST a. The concentration of produced LST a in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST a in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST a concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST a can be 40 g/l or greater, e.g., 50 g/l, 60 g/l, 70 g/l, 80 g/l, 90 g/l, or greater. For example, in some embodiments, concentrations of produced LST a in the culture medium can be 100 g/l or greater.

The yield of produced LST a on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST a on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST b. The concentration of produced LST b in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST b in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST b concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST b can be 40 g/l or greater, e.g., 50 g/l, 60 g/l, 70 g/l, 80 g/l, 90 g/l, or greater. For example, in some embodiments, concentrations of produced LST b in the culture medium can be 100 g/l or greater.

The yield of produced LST b on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST b on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST c. The concentration of produced LST c in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST c in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST c concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST c can be 40 g/l or greater, e.g., 50 g/l, 60 g/l, 70 g/l, 80 g/l, 90 g/l, or greater. For example, in some embodiments, concentrations of produced LST c in the culture medium can be 100 g/l or greater.

The yield of produced LST c on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST c on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DS-LNT. The concentration of produced DS-LNT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DS-LNT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DS-LNT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DS-LNT can be 40 g/l or greater, e.g., 50 g/l, 60 g/l, 70 g/l, 80 g/l, 90 g/l, or greater. For example, in some embodiments, concentrations of produced DS-LNT in the culture medium can be 100 g/l or greater.

The yield of produced DS-LNT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DS-LNT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LST a. The concentration of produced F-LST a in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LST a in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LST a concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LST a can be 40 g/l or greater, e.g., 50 g/l, 60 g/l, 70 g/l, 80 g/l, 90 g/l, or greater. For example, in some embodiments, concentrations of produced F-LST a in the culture medium can be 100 g/l or greater.

The yield of produced F-LST a on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LST a on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LST b. The concentration of produced F-LST b in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LST b in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LST b concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LST b can be 40 g/l or greater, e.g., 50 g/l, 60 g/l, 70 g/l, 80 g/l, 90 g/l, or greater. For example, in some embodiments, concentrations of produced F-LST b in the culture medium can be 100 g/l or greater.

The yield of produced F-LST b on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LST b on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FS-LNH. The concentration of produced FS-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FS-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FS-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FS-LNH can be 40 g/l or greater, e.g., 50 g/l, 60 g/l, 70 g/l, 80 g/l, 90 g/l, or greater. For example, in some embodiments, concentrations of produced FS-LNH in the culture medium can be 100 g/l or greater.

The yield of produced FS-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FS-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FS-LNnH. The concentration of produced FS-LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FS-LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FS-LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FS-LNnH can be 40 g/l or greater, e.g., 50 g/l, 60 g/l, 70 g/l, 80 g/l, 90 g/l, or greater. For example, in some embodiments, concentrations of produced FS-LNnH in the culture medium can be 100 g/l or greater.

The yield of produced FS-LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FS-LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FDS-LNH II. The concentration of produced FDS-LNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FDS-LNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FDS-LNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FDS-LNH II can be 40 g/l or greater, e.g., 50 g/l, 60 g/l, 70 g/l, 80 g/l, 90 g/l, or greater. For example, in some embodiments, concentrations of produced FDS-LNH II in the culture medium can be 100 g/l or greater.

The yield of produced FDS-LNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FDS-LNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

### Fermentation Compositions

Also provided are fermentation compositions including a population host cells. The host cells may include any of the yeast cells disclosed herein and discussed above. In some embodiments, the fermentation composition further includes at least one HMO. The HMO may be a reducing sugar. In some embodiments, the HMO contains a terminal lactose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

### Methods of Recovering HMOs

Also provided are methods of recovering one or more HMOs from a host cell or fermentation composition described herein. The method may include separating at least a portion of a population of host cells from a culture medium. In some embodiments, the separating includes centrifugation. In some embodiments, the separating includes filtration.

The provided recovery methods may further include contacting the separated host cells with a heated wash liquid. In some embodiments, the heated wash liquid is a heated aqueous wash liquid. In some embodiments, the heated wash liquid consists of water. In some embodiments, the heated wash liquid includes one or more other liquids or dissolved solid components.

In some embodiments, the method may further include removing the wash liquid from the host cells. In some embodiments, the removed wash liquid is combined with the separated culture medium and further processesed to isolate the one or more HMOs. In some embodiments, the removed wash liquid and the separated culture medium are further processed independently of one another. In some embodiments, the removal of the wash liquid from the yeast cells is accomplished by way of centrifugation. In some embodiments, the removal of the wash liquid from the yeast cells is accomplished by way of filtration.

### Infant Formula

Additionally described herein is an infant formula, particularly an infant formula produced by: (i) culturing any one of the host cells of the disclosure in a culture medium, thereby producing a desired HMO, (ii) extracting the HMO, and (iii) formulating the HMO for administration to an infant human subject. The infant formula may be in a liquid form as a concentrate or a ready-to-drink liquid. Alternatively, the infant formula may be in the form of a dry powder that may be reconstituted by the addition of water. The infant formula may be used as a human milk replacement or supplement. In some embodiments, the infant formula is formulated such that it is suitable for consumption by an infant of less than 2 years of age, such as an infant of 23 months or less, 22 months or less, 21 months or less, 20 months or less, 19 months or less, 18 months or less, 17 months or less, 16 months or less, 15 months or less, 14 months or less, 13 months or less, 12 months or less, 11 months or less, 10 months or less, 9 months or less, 8 months or less, 7 months or less, 6 months or less, 5 months or less, 4 months or less, 3 months or less, 2 months or less, or 1 month or less.

Also provided herein are methods of producing an infant formula using the host cells and fermentation compositions of the disclosure. The methods may include, for example, (i) culturing any one of the host cells of the disclosure in a culture medium, thereby producing a desired HMO, (ii) extracting the HMO, and (iii) formulating the HMO for administration to an infant human subject.

In some embodiments, the infant formula of the disclosure includes one or more HMOs selected from LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, and FDS-LNH II.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Identification and Deletion of Native Aldose Reductases in S. cerevisiae to Increase LNnT Yield and Purity

Biosynthesis of the human milk oligosaccharide (HMO) lacto-N-neotetraose (LNnT) in yeast can be achieved with the expression of just three heterologous enzymes: lactose permease (LAC12), β-1,3-N-acetylglucosaminyltransferase (LgtA), and β-1,4-galactosyltransferase (LgtB). One of the major challenges in producing LNnT, however, is to biosynthesize the HMO with high purity. An important source of impurities, addressed in this example, are sugar alditols produced by native aldose reductases in S. cerevisiae. These reductases compete with pathway enzymes for substrate, converting lactose and LNnT into lactitol and LNnT-alditol, respectively. In some instances, the reductases may act to hydrogenate other HMO byproducts to sugar alcohols. Sugar alcohols, such as LNnT-alditol, are not desirable in a final HMO product formulation. In this example, three rounds of native reductase knockout screening were performed in LNnT-producing yeast strains in an effort to minimize the formation of the sugar alcohol, lactitol. The results of these experiments are described in further detail below.

### Abbreviations

Lacto-N-triose II (LNTrII or LNT2)
Lacto-N-neotetraose (LNnT)
Para-Lacto-N-neopentaose (p-LNnP)
Para-Lacto-N-neohexaose (p-LNnH)
N-acetylglucosamine (GlcNAc, NAG)
UDP-N-acetylglucosamine (UDP-GlcNAc)
UDP-Galactose (UDP-Gal)
β-1,3-N-acetylglucosaminyltransferase (LgtA)
β-1,4-galactosyltransferase (LgtB)

### Overview

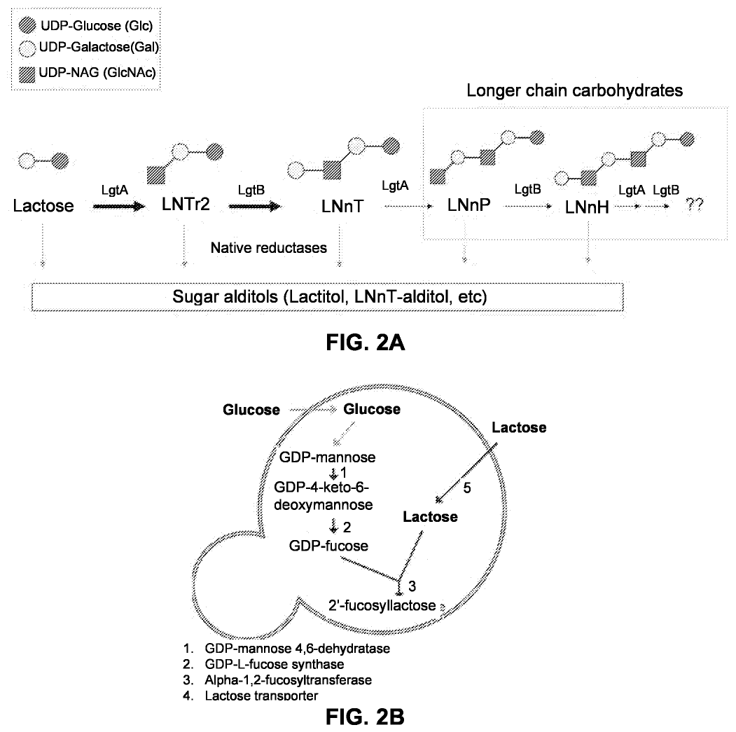

Heterologous production of LNnT in yeast requires three non-native enzymes: Lactose permease (LAC12 from K. lactis) to import fed lactose, LgtA (β-1,3-N-acetylglucosaminyltransferase) to convert lactose to LNT2, and LgtB to convert LNT2 to LNnT (FIG. 2A). Despite the simplicity of the pathway, product purity is a major challenge for LNnT. It is shown in this example that a key reason for the production of impurities during the LNnT biosynthetic process is the conversion of lactose-based molecules into their respective sugar alditols by the activity of native yeast reductases. The experiments conducted in this example further demonstrate that by diminishing the expression and/or activity of such native reductases, HMOs, such as LNnT, can be produced in higher overall yield and with elevated purity by virtue of suppressing the formation of sugar alditols.

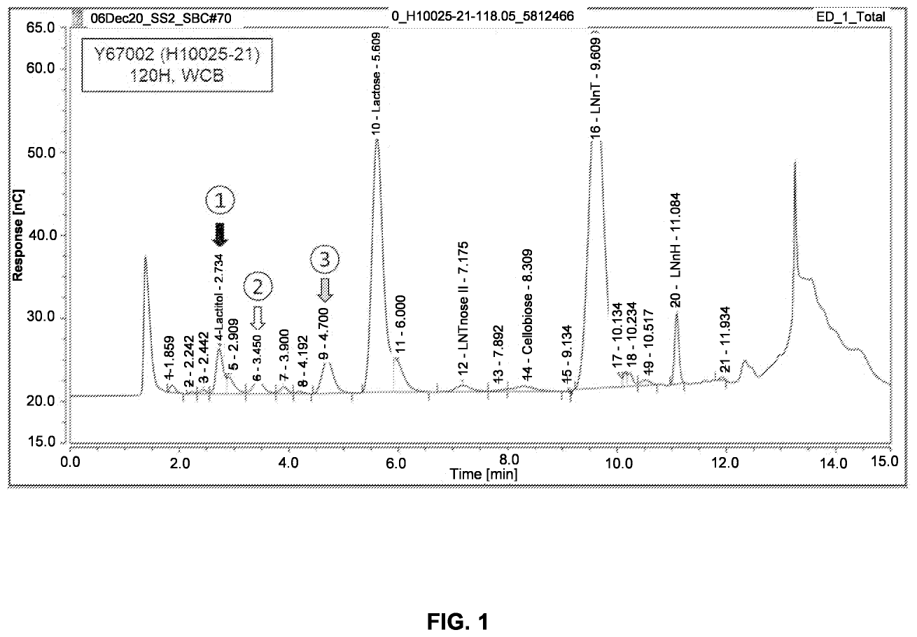

FIG. 1 shows a typical ion chromatography trace for a top LNnT strain Y67002. Peak 1 is lactitol, as confirmed and quantified via a commercial standard. Peak 2 is likely LNT2-alditol. Peak 3 is referred to herein as “Unknown A2” and is a co-elution of two peaks, one of which is likely to be LNnT-alditol. Reference material for LNnT-alditol was used to confirm that LNnT-alditol was indeed one of the peaks in Unknown A2.

As is shown in FIG. 2A, native reductases compete with HMO biosynthesis pathway enzymes for access to substrate molecules. The experiments conducted in this example demonstrate that conversion of HMO biosynthesis pathway products into sugar alditols reduces overall yield. At the outset, however, it was unknown which, or how many, reductases were responsible for competing with HMO biosynthesis pathway enzymes for substrate.

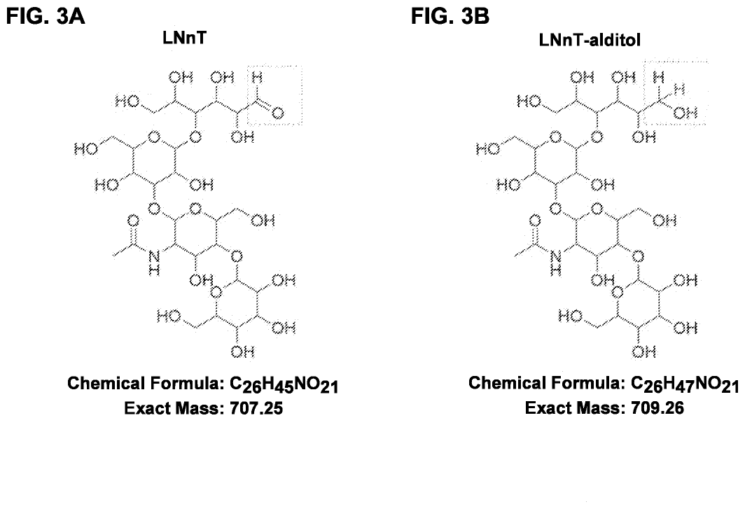

FIGS. 3A and 3B show the chemical structure of LNnT as compared to that of LNnT-alditol. The conversion of aldose sugars to alditols results from the hydrogenation of the aldehyde substituent at the end of the lactose moiety, a process mediated by aldose reductases. Aldose reductases are a class of the oxidoreductase enzyme family with reducing activity on aldose sugars. In this example, a multi-tier strategy was utilized to screen for native yeast enzymes that may have aldose reductase activity toward lactose and LNnT.

In Tier 1, 28 reductases in a lactose-permease-expressing yeast strain were deleted. In this tier, a decrease in the production of lactitol upon deletion of a candidate reductase was used as the primary readout of reductase activity for the candidate being tested. Genes identified as encoding a reductase having activity toward lactose from Tier 1 were then validated in a LNnT-producing yeast strain. As is described in further detail below, the top hit from Tier 1 was GCY1. In Tier 2, 6 reductases were knocked out in the gcy1 deletion (gcy1Δ) yeast strain, and a second additive deletion was identified (gre3A). In Tier 3, 10 reductases were deleted from the double-deletion (gcy1Δ, gre3A) strain, and two additional hits (adh6Δ, sfa1Δ) were identified as achieving LNnT production with reduced production of lactitol.

The results of these experiments are described in further detail in the sections below.

### Materials and Methods

### Microtiter Plate Growth Conditions

Pre-culture growth: Strains were incubated in an aerobic pre-culture 96-well 1.1 ml microtiter shake-plate at 28° C., shaking at 1,000 RPM for 48h to reach carbon exhaustion. Pre-culture media conditions included 360 μl/well of minimal complete media with 2% carbon (1.9% maltose+0.1% glucose) and 1 g/L Lysine.

Production growth: After pre-culture, strains were diluted approximately 10-fold (14.4 μl) into 130 μl/well 96-well 1.6 ml microtiter shake-plates containing 4% sucrose and 0.1% lactose. Production plates were incubated at 33.5° C., shaking at 1,000 RPM for 96h.

### Extraction Conditions for Ion Exchange Chromatography

After 4 days in production conditions, carbon-exhausted whole cell broth was extracted using a hot water method. Following the addition of 300 μl/well of sterile water, the plate was heated and mixed at 1000 RPM for 30 minutes. Plates were then centrifuged for 5 minutes at 2000 RPM. Finally, 25 μl/well of the supernatant were added to 175 μl/well of sterile water into a 1.6 ml plate (8×). The total dilution from whole cell broth was 40×.

### Analytics: Ion Exchange Chromatography (IC)

LNT, LNnT, LNTriose II, and Lactose were quantitated using external calibration and ion chromatography pulse amperometric detection with a Dionex® CarboPac® PA1 column. The analytical system used was the HPAEC-PAD: High-Performance Anion-Exchange Chromatography coupled with Pulse Electrochemical Detection using a Thermo Dionex® Ion Chromatography system. Columns used were CarboPac® PA1 (4×250 mm), CarboPac® PA1 guard (4×50 mm), lonPac® NG1 guard (4×35 mm). Analytes measured in this method were lactose, LNnT, LNTrII, p-LNnH, and lactitol. The matrix was fermentation broth diluted with water. Calibration type was 4-point with external standards for LNT, LNnT, LNnH, LNTriose II, Lactose and Lactitol.

### Results

### Tier 1: 28 Native Reductases in LAC12-Only Parent

The first round of the reductase knockout screen was performed in Y46373 background yeast strains. In a 0.1% lactose plate model, this strain produces −0.06 g/kg of lactitol. The signal sought in this round of reductase knockout screening was a decrease in the production of lactitol, as assessed by IC. This signal, when observed, was indicative of a candidate enzyme having reductase activity toward lactose. A total of 28 native reductases were deleted in the first screen (Table 2). Seven reductases were specifically identified as having reductase activity.

The reductases screened in this example are summarized in Table 2, below.

TABLE 2
All native yeast reductases screened in this study

| | Common | | Tier | Tier | Tier |
|---|---|---|---|---|---|
| Reductase | name | Description | 1 | 2 | 3 |
| YDR368W | YPR1 | Yeast Putative Reductase | x | x | x |
| YJR096W | | | x | x | x |
| YOL151W | GRE2 | Genes de Respuesta a | x | x | x |
| | | Estres (stress responsive | | | |
| | | genes) | | | |
| YBR149W | ARA1 | D-Arabinose | x | x | x |
| | | dehydrogenase | | | |
| YDL124W | | NADPH-dependent | x | x | x |
| | | alpha-keto amide | | | |
| | | reductase | | | |
| YHR104W | GRE3 | Genes de Respuesta a | x | x | |
| | | Estres (stress responsive | | | |
| | | genes) | | | |
| YOR120W | GCY1 | Glycerol dehydrogenase | x | | |
| YAL060W | BDH1 | Butanediol | x | | |
| | | Dehydrogenase | | | |
| YAL061W | BDH2 | | | | |
| YGL256W | ADH4 | Alcohol Dehydrogenase | x | | |
| YER081W | SER3 | Serine requiring | x | | |
| YFL056C | AAD6 | Aryl-Alcohol | x | | |
| | | Dehydrogenase | | | |
| YFL057C | AAD16 | Aryl Alcohol | x | | |
| | | Dehydrogenase | | | |
| YGL039W | | | x | | |
| YGL157W | ARI1 | Aldehyde Reductase | x | | |
| | | Intermediate, subclass | | | |
| | | of SDR | | | |
| YBR145W | ADH5 | Alcohol Dehydrogenase | x | | |
| YIL074C | SER33 | Serine requiring | x | | |
| YIR035C | NRE1 | Novel Reductase | x | | |
| YIR036C | IRC24 | Increased Recombination | x | | |
| | | Centers | | | |
| YKL107W | | | x | | |
| YLR174W | IDP2 | Isocitrate | x | | |
| | | Dehydrogenase, | | | |
| | | NADP-specific | | | |
| YML131W | | | x | | |
| YDL168W | SFA1 | Sensitive to | x | | |
| | | Formaldehyde | | | |
| YNL134C | | | x | | |
| YNL331C | AAD14 | Aryl-Alcohol | x | | |
| | | Dehydrogenase | | | |
| YPL061W | ALD6 | Aldehyde Dehydrogenase | x | | |
| YMR169C | ALD3 | Aldehyde Dehydrogenase | x | | |
| YMR318C | ADH6 | Alcohol dehydrogenase | x | | x |
| YOL086C | ADH1 | Alcohol dehydrogenase | | | x |
| YDL168W | SFA1 | Alcohol dehydrogenase | | | x |
| YGL256W | ADH4 | Alcohol dehydrogenase | | | x |
| YBR145W | ADH5 | Alcohol dehydrogenase | | | x |

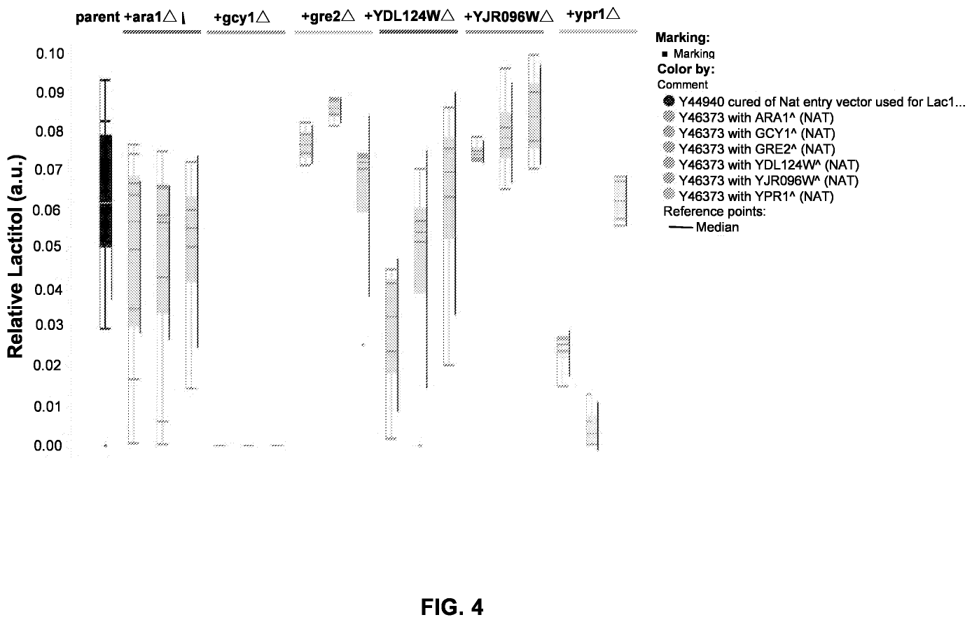

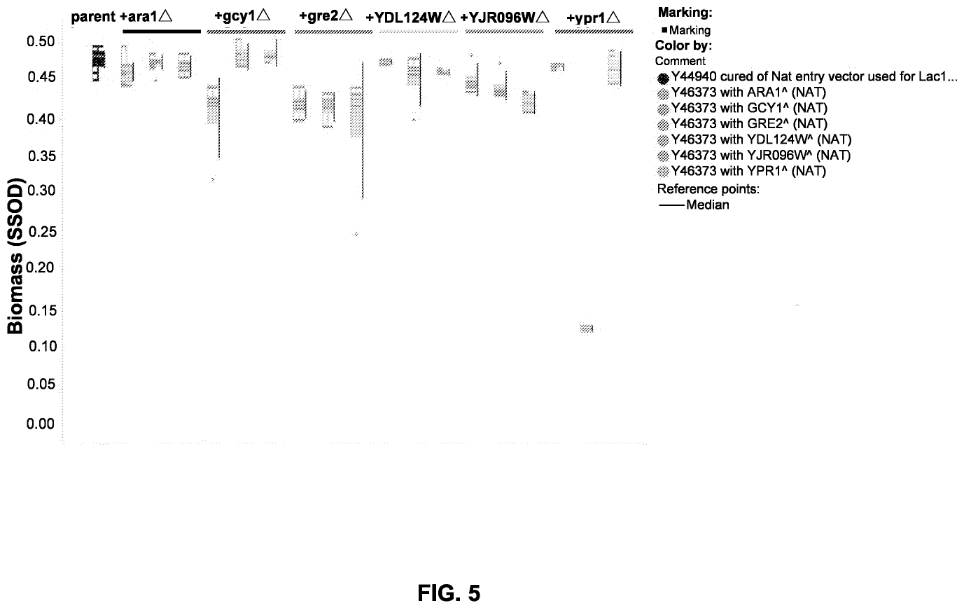

Y46373 yeast strains having a gcy1 deletion exhibited no detectable lactitol in all 3 cPCR-confirmed isolates, and Y46373 yeast strains having a ypr1 deletion had significant reduction in the lactitol levels in 2 out of 3 cPCR-confirmed isolates (FIG. 4). Interestingly, gcy1 (glycerol dehydrogenase) and ypr1 (NADPH-dependent aldo-keto reductase) are paralogs, and both genes are inducible by galactose. Biomass (ssOD) measurements obtained from the reductase knockout strains are shown in FIG. 5.

After the first round of the reductase screen, both gcy1 and ypr1 deletions were tested in the Y67002 yeast strain, which is the highest-producing of the LNnT strains examined in this example. Y67002 with gcy1Δ (“Y68726”) exhibited an improved product profile compared to Y67002, with lower lactitol and “Unknown A2” levels. The Unknown A2 peak was later shown to consist of LNnT-alditol and at least one other unknown molecule(s).

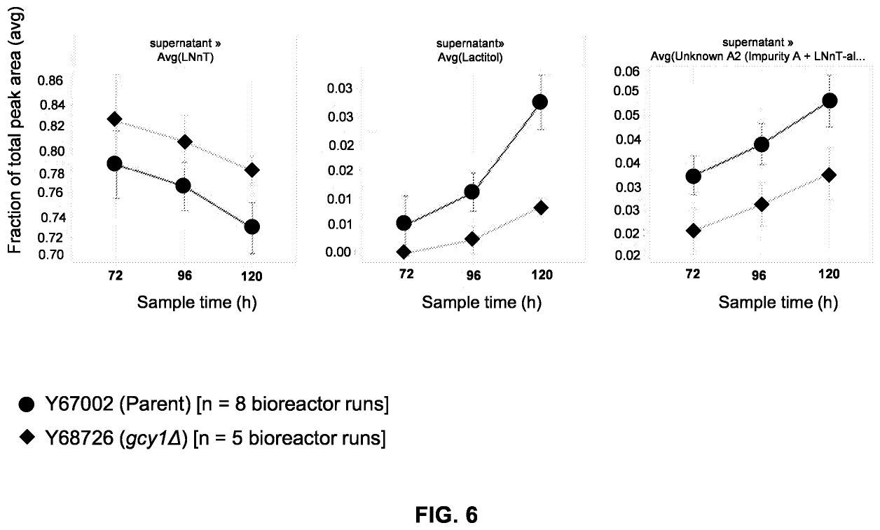

Following this result, the product profile of the Y68726 strain was evaluated in 0.25-L tanks. In this context, Y68726 showed a significant, 4-5% increase in LNnT peak area percentage when compared to parent (FIG. 6), as assessed by IC. This increase in LNnT production appeared to be a result of reduced formation of lactitol (−2%) and LNnT-alditol (part of Unknown A2).

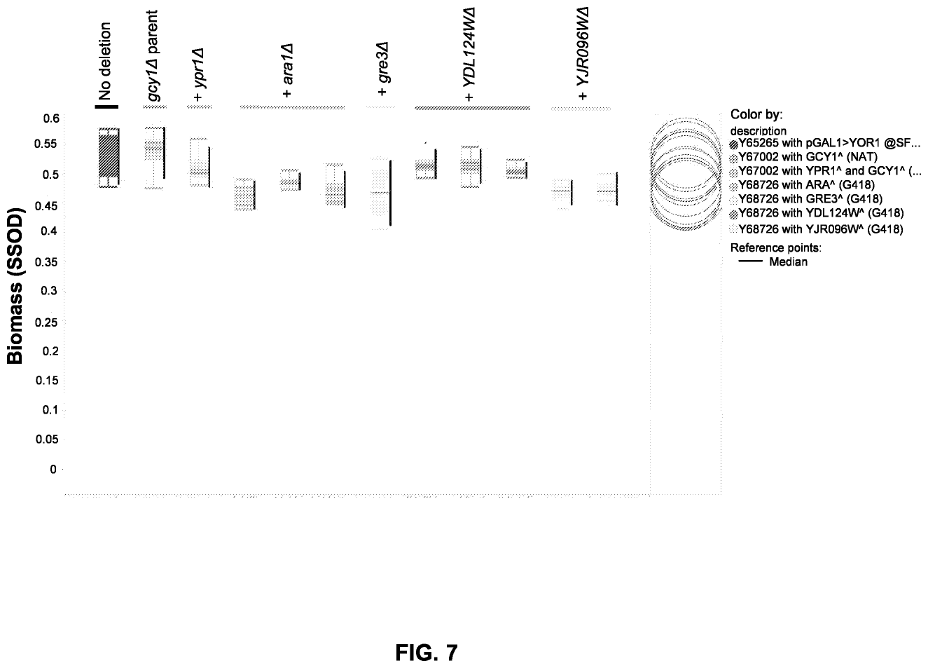

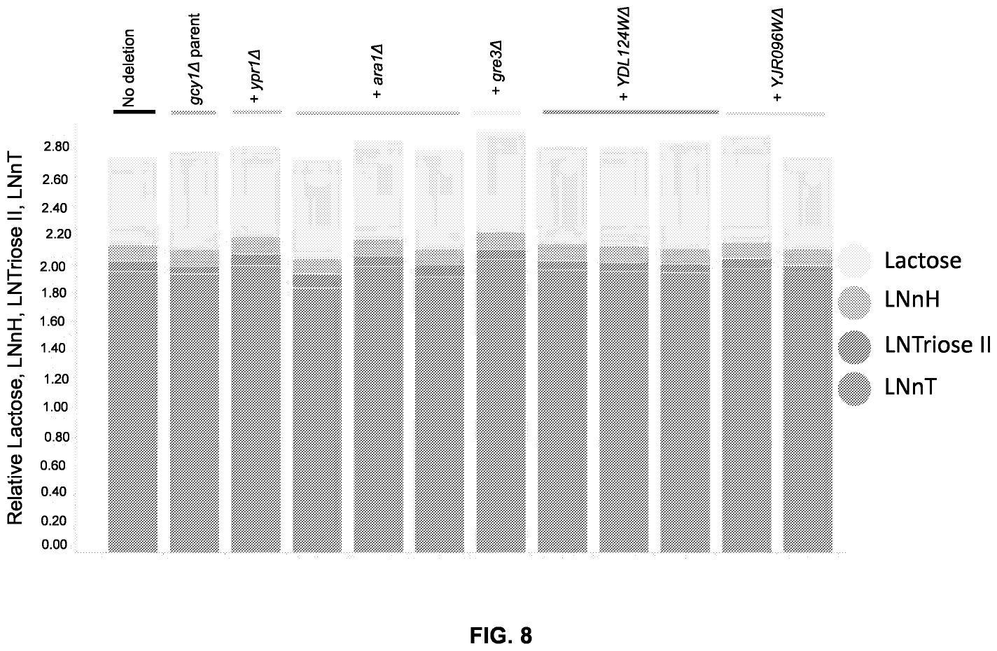

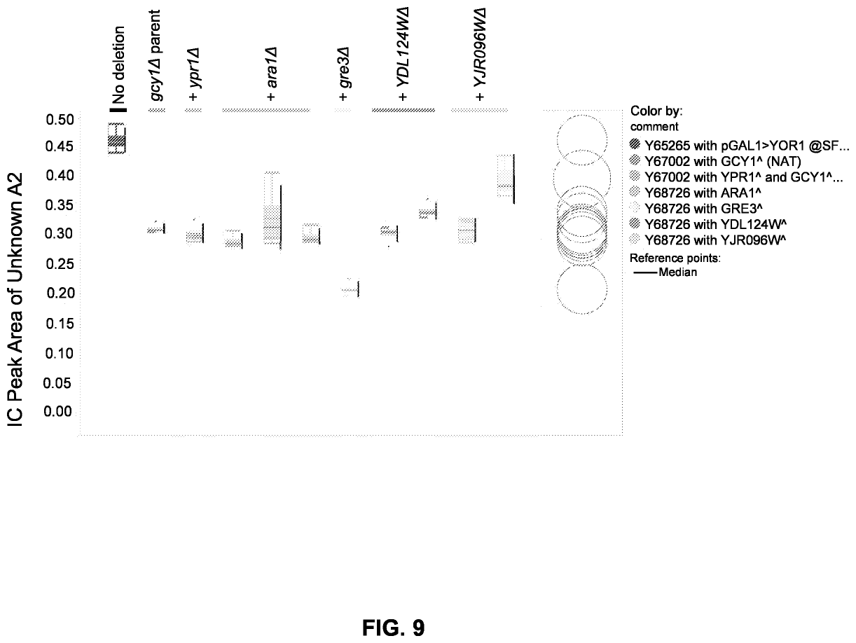

In the second round, 6 of the 28 reductases in the gcy1Δ strain background (Y68726) were re-screened (Table 2). When screened in the context of gcy1Δ, gre3 was determined to be a deletion hit. The resulting strain was “Y70141” (Y67002+gcy1Δ, gre3Δ). Since the parent strain was already free of all lactitol signal, the following signals were used as indicators of reduced alditol conversion: increased lactose titer, increased LNT2 titer, and increased LNnT titer. A significant change in titers was not observed for any of these three molecules compared to parent strains when tested in plates (FIG. 7 and FIG. 8). However, Y70141 did exhibit reduced “Unknown A2” peak area compared to the parent (FIG. 9). When compared to the gcy1Δ parent, only the additional gre3A showed further reduction of “Unknown A2” peak area.

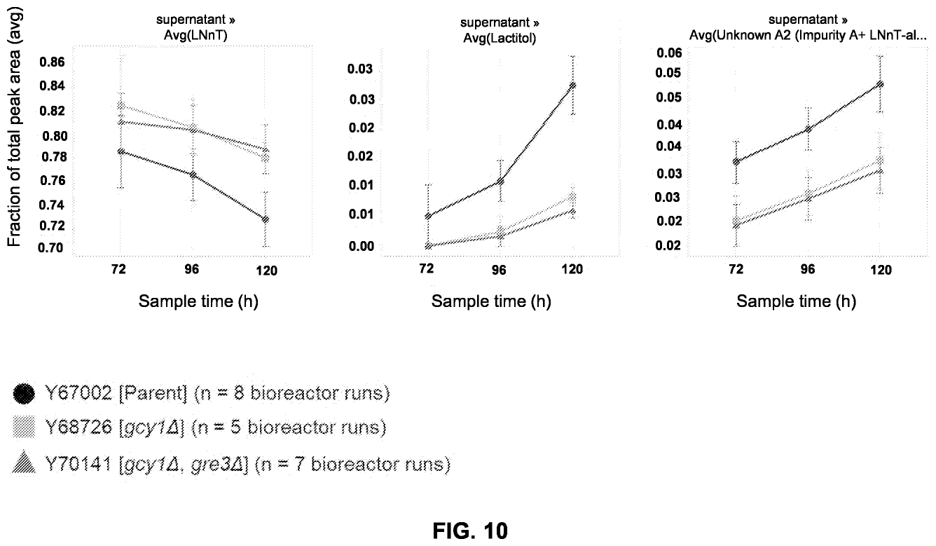

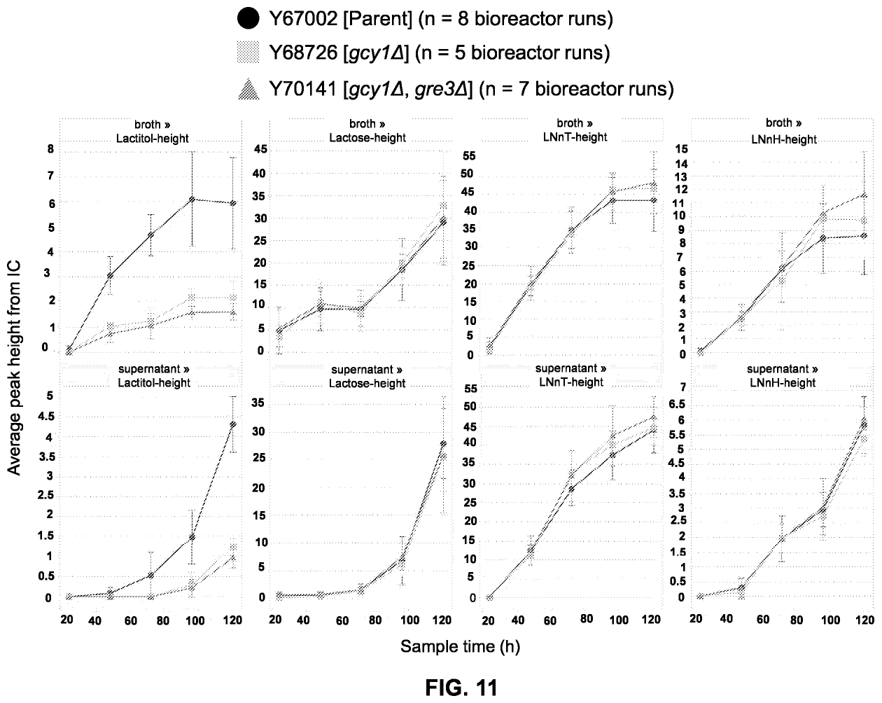

Testing of the double-deletion strain, Y70141 (gcy1Δ, gre3Δ), in 0.25-L fermentation tanks resulted in a decrease in median peak area percent for lactitol and “Unknown A2” over the single reductase deletion strain (FIG. 10). The peak area of LNnT effectuated by the Y70141 strain was similar to that of the single-deletion strain, Y68726 (gcy1Δ). A comparison of titer data between the two strains shows a small increase in average LNnT and p-LNnH titer for Y70141 (FIG. 11). Y70141 also exhibits an increase in p-LNnH titers, a result that may be due to more available LNnT and/or to a reduction in the production of LNnH-alditol (FIG. 11).

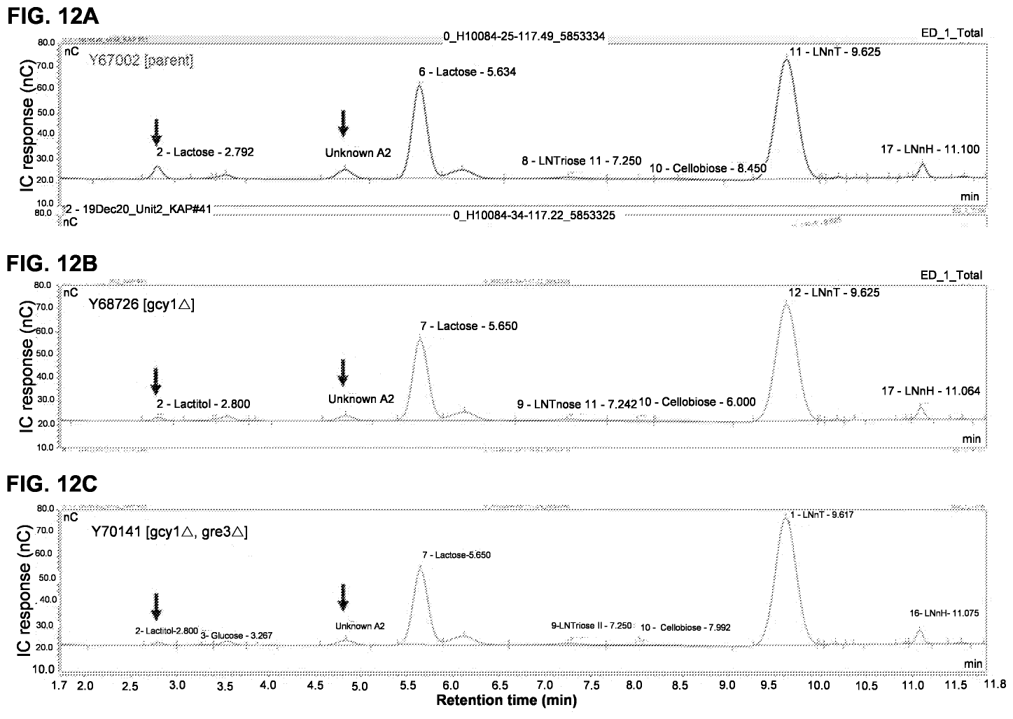

FIGS. 12A-12C show IC traces from 0.25-L bioreactor analyses of Y67002, Y68726, and Y70141 strains. In each case, lactitol and “Unknown A2” peaks are indicated by black arrows. Y68726 (gcy1Δ) exhibited decreased production of lactitol and “Unknown A2” (FIG. 12B). Y70141 (gcy1Δ, gre3Δ) also demonstrated decreased lactitol and “Unknown A2” production (FIG. 12C). The deletion of gcy1 accounted for most of the decrease in lactitol and “Unknown A2” peak areas. The difference between gcy1 and gre3 deletion is more subtle, as there is not a clear difference in lactitol or “Unknown A2” peak areas between the Y68726 and Y70141 strains.

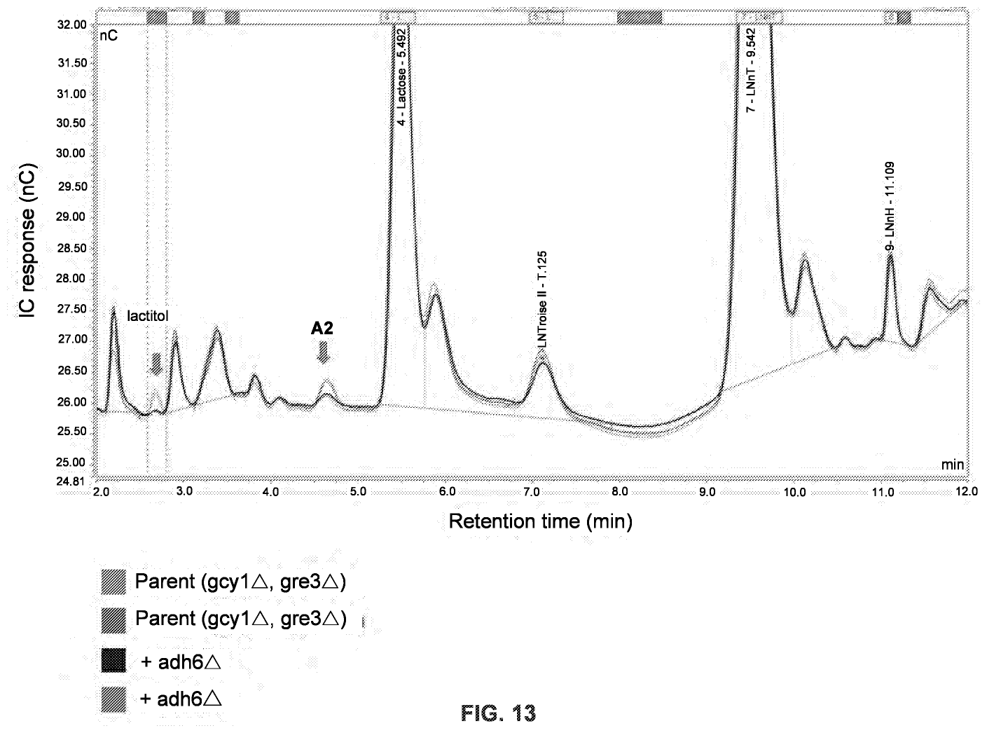

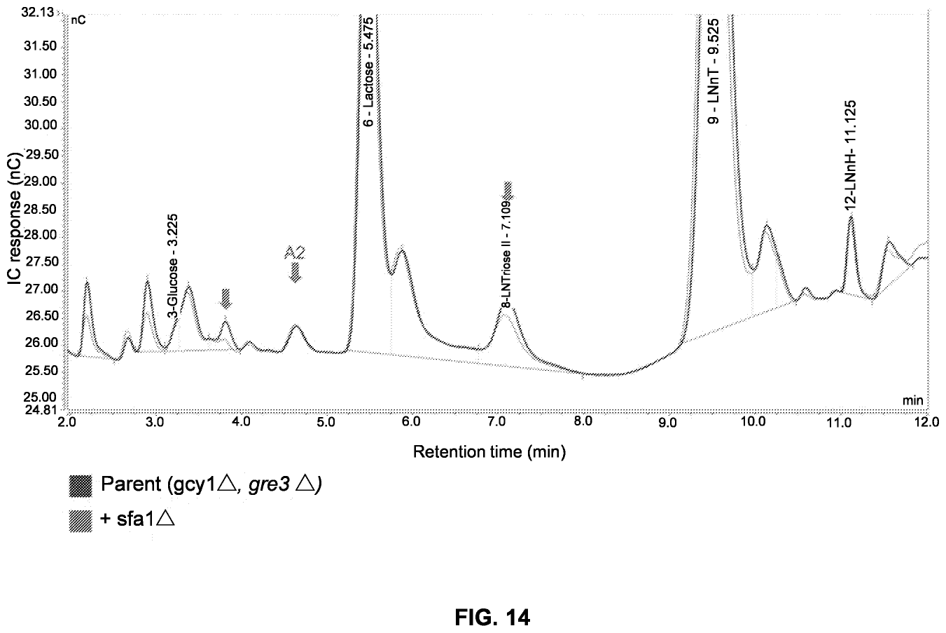

After conducting the first two tiers of reductase knockout screening, a third tier of screening was performed. In the third tier, the double-deletion strain, Y70141 (gcy1Δ, gre3Δ), was used as the screening background. In tier 3, the remaining reductases from tier 2 were re-screened, along with five other putative alcohol dehydrogenases (Table 2). Ultimately, there were 2 additional reductase hits from the third round of screening: ADH6 and SFA1. The “Y71152” strain (gcy1Δ, gre3Δ, adh6Δ) exhibited lower “Unknown A2” production compared to the double-deletion parent (FIG. 13). Similarly, the “Y71148” strain (gcy1Δ, gre3Δ, sfa1Δ) exhibited a reduction in the production of an impurity eluting at about the 3.9-minute retention timepoint compared to the double-deletion parent (FIG. 14).

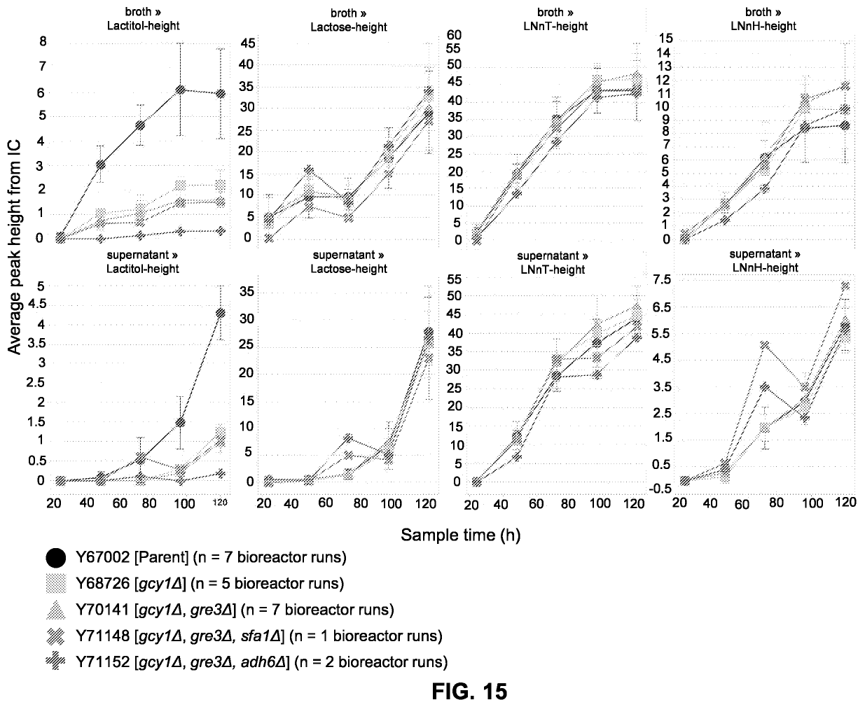

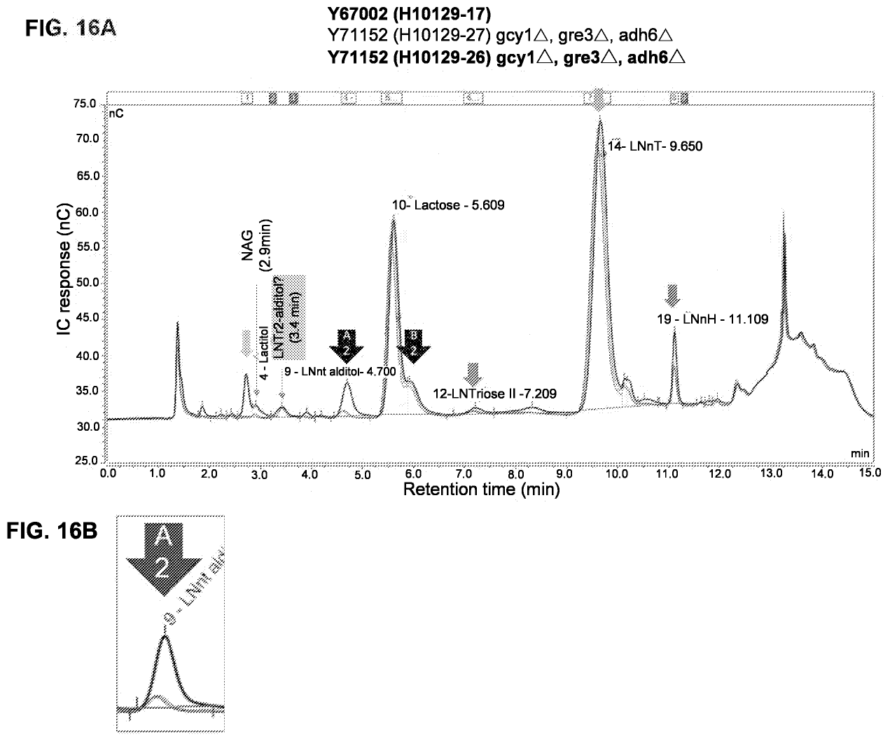

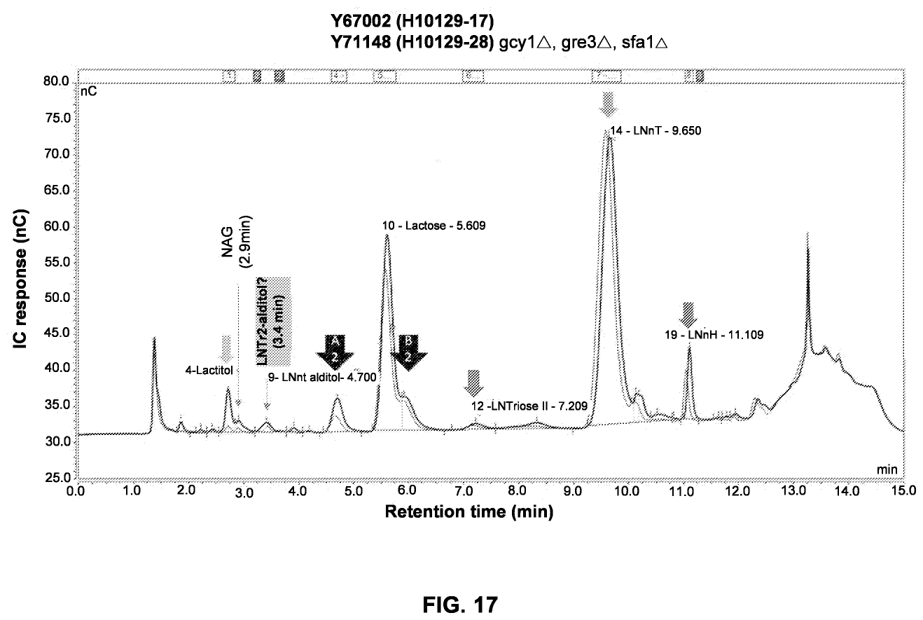

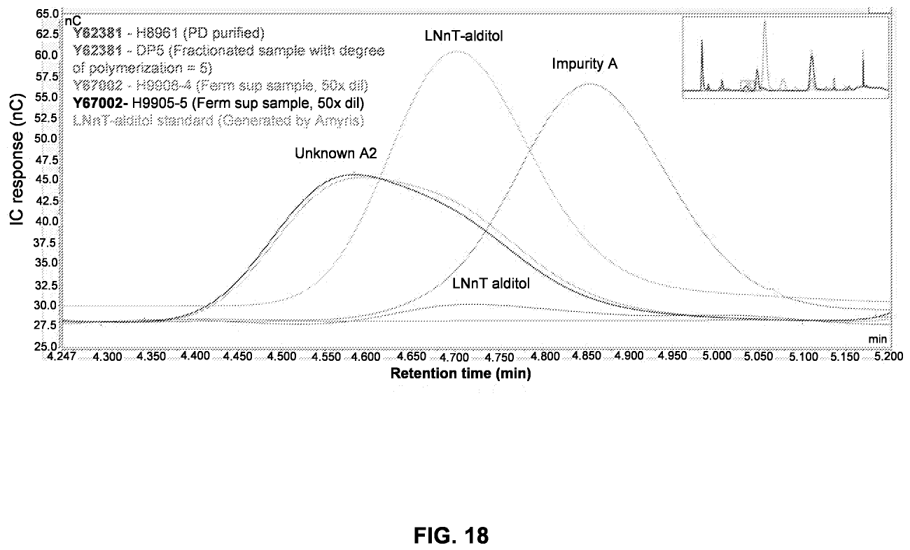

Following the tier 3 screen, both Y71152 and Y71148 were evaluated at 0.25-L scale in fermentation tanks. The results of these experiments are shown in FIG. 15. Neither triple reductase knockout strain outperformed the double reductase knockout parent. LNnT titers for triple reductase strains Y71148 and Y71152 were lower than parent Y70141 in both broth and supernatant samples as is shown in FIGS. 16, 17, and 18 and summarized in FIG. 15.

### Conclusions

The two reductases found to have significant activity toward lactose and LNnT were GCY1 and GRE3. The deletion of GCY1 and GRE3 reductases led to an improvement in LNnT product purity and increase in LNnT titer. Based on the results of this study, and using the compositions and methods described herein, host cells (e.g., yeast strains) having deficiencies in expression and/or activity of one or more of the reductases analyzed above, such as GCY1 and/or GRE3, can be produced and utilized so as to biosynthesize HMOs (e.g., LNnT) in high titer and with improved product purity by suppressing the formation of undesirably sugar alditol byproducts.

### Example 2. Deletion of Gcy1 in S. cerevisiae to Increase 2′-Fucosyllactose (2′-FL) Yield and Purity

As Example 1 demonstrates, knocking out native aldose reductase genes gcy1 and gre3 resulted in improved lacto-N-neotetraose (LNnT) purity and titer. In this example, the deletion of native gcy1 in S. cerevisiae was evaluated for the ability to reduce the production undesired 2′-fucosyllactitol, thereby increasing the yield and purity of another HMO: 2′-fucosyllactose (2′-FL), which is made according the biosynthetic pathway described in FIG. 2B.

### Materials and Methods

The materials and methods used in this example mirrored those used in Example 1, but were adapted for the detection of 2′FL and 2′-fucosyllactitol.

### Results

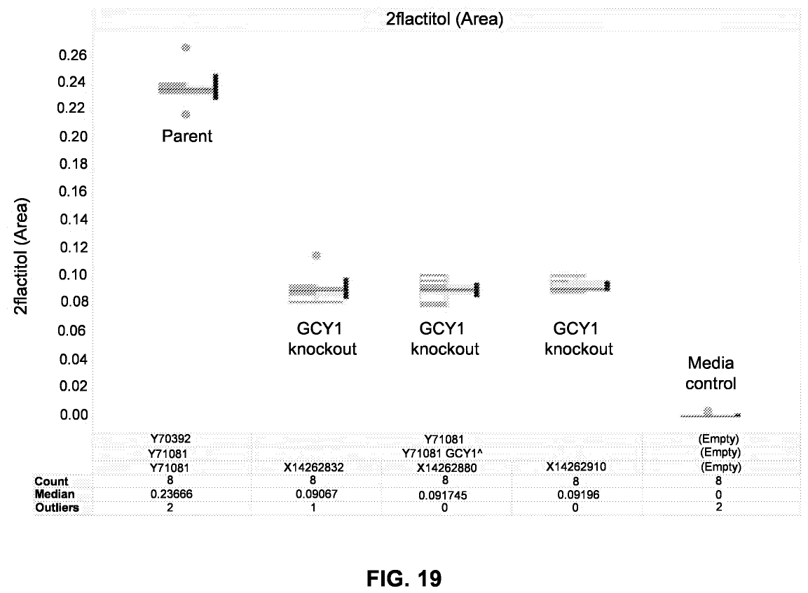

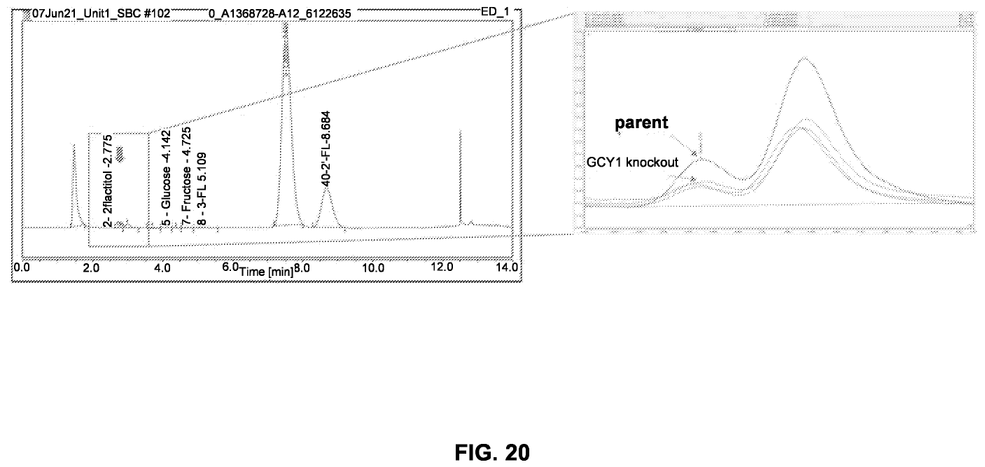

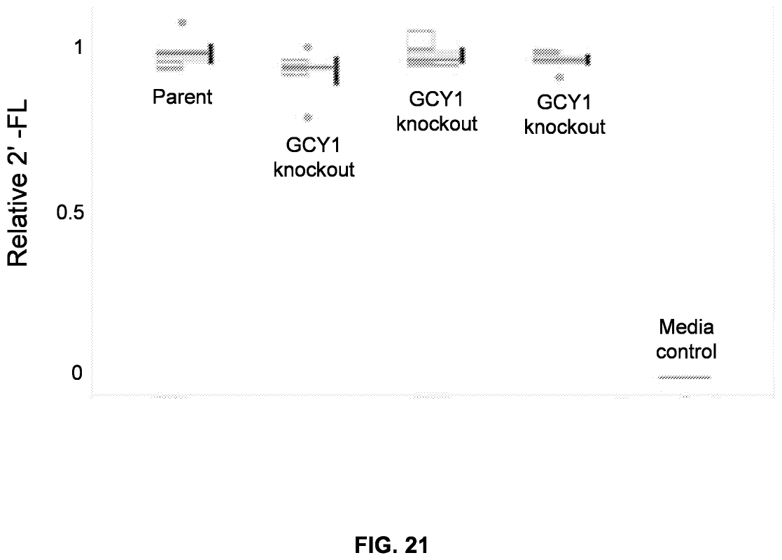

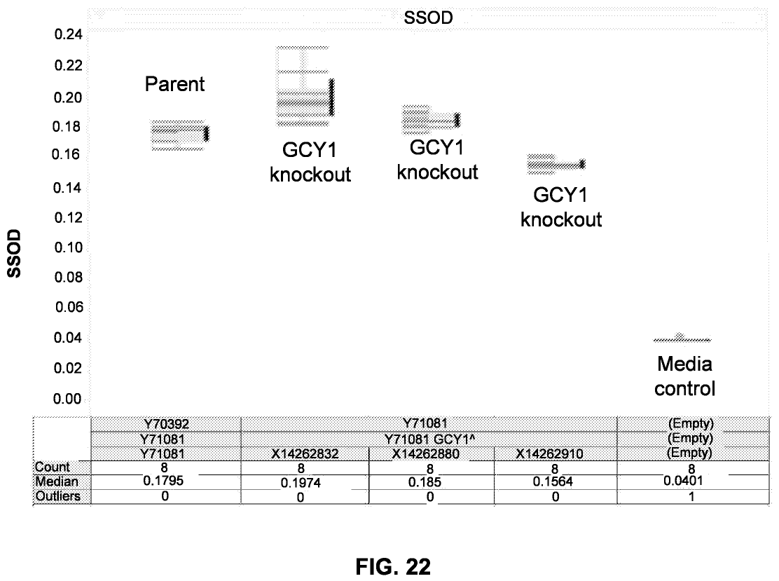

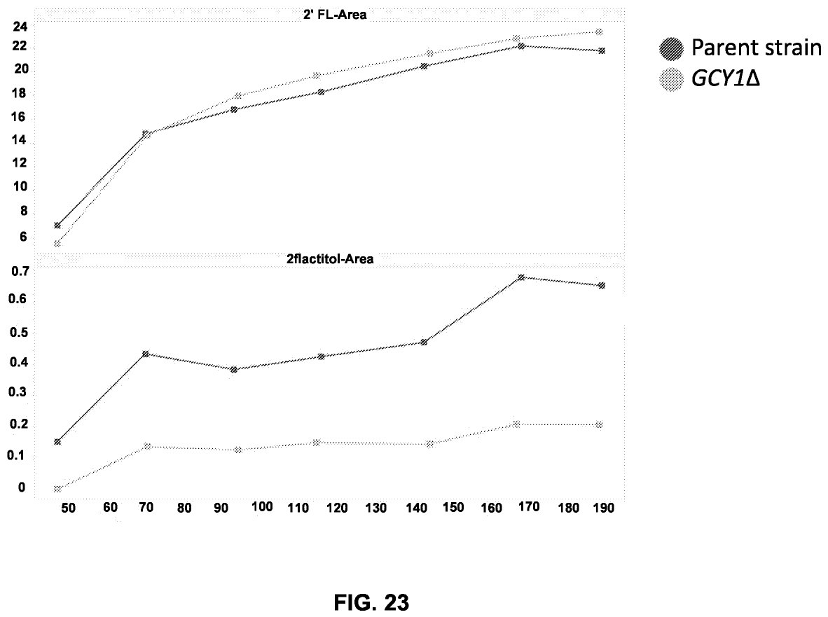

Knocking out gcy1 from a 2′-FL-producing yeast strain, Y71081, led to an approximately 2.6 fold reduction of 2′-fucosyllactitol peak area in three cPCR confirmed clones compared to an unmodified version of Y71081 lacking the reductase knockout (FIG. 19). Chromatogram overlays of the parent product profile compared to that of the gcy1 knockout show a distinct reduction in peak size corresponding to 2′-fucosyllactitol (FIG. 20). Interestingly, gcy1 knockout also reduced the size of a neighboring impurity peak. 2′-FL titer (FIG. 21) and biomass (FIG. 22) measured using SSOD were not affected by gcy1 deletion. Testing of the reductase knockout strain in 0.25-L fermentation tanks resulted in a decrease in peak area for 2′-fucosyllactitol and slight increase in 2′-FL peak area from the parent Y71081 (FIG. 23).

### Conclusions

The results from this example, taken together with those from Example 1, demonstrate that suppressing the activity and/or expression of an endogenous oxidoreductase (e.g., an endogenous aldose reductase), such as GCY1, in a HMO-producing host cell not only augments the titer and purity of biosynthesized LNnT, but also improves the titer and purity of another HMO: 2-FL. Guided by the results of these studies, and using the compositions and methods described herein, host cells (e.g., yeast strains) having deficiencies in expression and/or activity of one or more of the reductases analyzed above, such as GCY1 and/or GRE3, can be produced and utilized so as to improve the yield and purity of a variety of HMOs having a terminal lactose, such as LNnT, 2′-FL, and others, including, e.g., 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, and fucosyl-disialyllacto-N-hexaose (FDS-LNH) II.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

### Sequence Appendix

Wildtype Neisseria meningitidis LgtA
β1-1,3-N-acetylglucosaminyltransferase
SEQ ID NO: 1
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_A258D
SEQ ID NO: 2
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVSSKYSIRQHEIAQGIQKTDRNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_c.945delA
SEQ ID NO: 3
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPMPVPG*
Nme.LgtA_E294N.c890addT
SEQ ID NO: 4
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPNEDFRKS*
Nme.LgtA_G179R
SEQ ID NO: 5
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFRNPIHNNTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_K242H
SEQ ID NO: 6
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVSSHYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_L229P
SEQ ID NO: 7
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEAPVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_M187P
SEQ ID NO: 8
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTPIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_N185G
SEQ ID NO: 9
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNGTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_P89T and G179R
SEQ ID NO: 10
MPSEAFRRHRAYRENKLQSLVSVLICAYNVEKYFAQSLAAVVNQTWRNLEILIVDDGSTDGTLAIAKDFQK
RDSRIKILAQAQNSGLITSLNIGLDELAKSGMGEYIARTDADDIAAPDWIEKIVGEMEKDRSIIAMGAWLE
VLSEEKDGNRLARHHRHGKIWKKPTRHEDIADFFPFRNPIHNNTMIMRRSVIDGGLRYNTERDWAEDYQFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_Q211V
SEQ ID NO: 11
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYVFW
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_S240V
SEQ ID NO: 12
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFW
YDVSKLGRLAYYPEALVKYRLHANQVVSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
Nme.LgtA_W213N
SEQ ID NO: 13
MPSEAFRRHRAYRENKLQSLVSVLICAYNVEKYFAQSLAAVVNQTWRNLEILIVDDGSTDGTLAIAKDFQK
RDSRIKILAQAQNSGLIPSLNIGLDELAKSGMGEYIARTDADDIAAPDWIEKIVGEMEKDRSIIAMGAWLE
VLSEEKDGNRLARHHRHGKIWKKPTRHEDIADFFPFGNPIHNNTMIMRRSVIDGGLRYNTERDWAEDYQFN
YDVSKLGRLAYYPEALVKYRLHANQVSSKYSIRQHEIAQGIQKTARNDFLQSMGFKTRFDSLEYRQIKAVA
YELLEKHLPEEDFERARRFLYQCFKRTDTPPAGAWLDFAADGRMRRLFTLRQYFGILRRLLKNR*
LAC12 lactose permease from K.lactis
SEQ ID NO: 14
MADHSSSSSSLQKKPINTIEHKDTLGNDRDHKEALNSDNDNTSGLKINGVPIEDAREEVLLPGYLSKQYYK
LYGLCFITYLCATMQGYDGALMGSIYTEDAYLKYYHLDINSSSGTGLVFSIFNVGQICGAFFVPLMDWKGR
KPAILIGCLGVVIGAIISSLTTTKSALIGGRWFVAFFATIANAAAPTYCAEVAPAHLRGKVAGLYNTLWSV
GSIVAAFSTYGTNKNFPNSSKAFKIPLYLQMMFPGLVCIFGWLIPESPRWLVGVGREEEAREFIIKYHLNG
DRTHPLLDMEMAEIIESFHGTDLSNPLEMLDVRSLFRTRSDRYRAMLVILMAWFGQFSGNNVCSYYLPTML
RNVGMKSVSLNVLMNGVYSIVTWISSICGAFFIDKIGRREGFLGSISGAALALTGLSICTARYEKTKKKSA
SNGALVFIYLFGGIFSFAFTPMQSMYSTEVSTNLTRSKAQLLNFVVSGVAQFVNQFATPKAMKNIKYWFYV
FYVFFDIFEFIVIYFFFVETKGRSLEELEVVFEAPNPRKASVDQAFLAQVRATLVQRNDVRVANAQNLKEQ
EPLKSDADHVEKLSEAESV
LgtB from Pasteurella multocida
SEQ ID NO: 15
MSGEHYVISLSSAVERRQHIRNQFSQKNIPFQFFDAISPSPLLDQLVLQFFPRLADSSLTGGEKACFMSHL
SLWHKCVEENLPYIVVFEDDIVLGKDADKFLIGDEWLFSRFDPEEIFIIRLETFLQKVVCESTHIAPYTHR
DFLSLKSAHFGTAGYVISQGAAKFLLDIFKNISNEHIAPIDELIFNQFLVKNSFNVYQLSPAICVQELQLN
NESSALQSQLELERNKFRNKKSEELKRNRKNFIEKFIYILKKPKRMLDNNKRKREESKIENDKMIIEFK
LgtB from Neisseria gonorrhoeae
SEQ ID NO: 16
MQNHVISLASAAERRAHIAATFGSRGIPFQFFDALMPSERLERAMAELVPGLSAHPYLSGVEKACFMSHAV
LWEQALDEGVPYIAVFEDDVLLGEGAEQFLAEDTWLQERFDPDSAFVVRLETMFMHVLTSPSGVADYGGRA
FPLLESEHCGTAGYIISRKAMRFFLDRFAVLPPERLHPVDLMMFGNPDDREGMPVCQLNPALCAQELHYAK
FHDQNSALGSLIEHDRRLNRKQQWRDSPANTFKHRLIRALTKIGREREKRRQRREQLIGKIIVPFQ
Lac12 from Kluyveromyces lactis
SEQ ID NO: 17
MADHSSSSSSLQKKPINTIEHKDTLGNDRDHKEALNSDNDNTSGLKINGVPIEDAREEVLLPGYLSKQYYK
LYGLCFITYLCATMQGYDGALMGSIYTEDAYLKYYHLDINSSSGTGLVFSIFNVGQICGAFFVPLMDWKGR
KPAILIGCLGVVIGAIISSLTTTKSALIGGRWFVAFFATIANAAAPTYCAEVAPAHLRGKVAGLYNTLWSV
GSIVAAFSTYGTNKNFPNSSKAFKIPLYLQMMFPGLVCIFGWLIPESPRWLVGVGREEEAREFIIKYHLNG
DRTHPLLDMEMAEIIESFHGTDLSNPLEMLDVRSLFRTRSDRYRAMLVILMAWFGQFSGNNVCSYYLPTML
RNVGMKSVSLNVLMNGVYSIVTWISSICGAFFIDKIGRREGFLGSISGAALALTGLSICTARYEKTKKKSA
SNGALVFIYLFGGIFSFAFTPMQSMYSTEVSTNLTRSKAQLLNFVVSGVAQFVNQFATPKAMKNIKYWFYV
FYVFFDIFEFIVIYFFFVETKGRSLEELEVVFEAPNPRKASVDQAFLAQVRATLVQRNDVRVANAQNLKEQ
EPLKSDADHVEKLSEAESV
GDP-mannose 4,6-dehydratase from Escherichia coli
SEQ ID NO: 18
MSKVALITGVTGQDGSYLAEFLLEKGYEVHGIKRRASSFNTERVDHIYQDPHTCNPKFHLHYGDLSDTSNL
TRILREVQPDEVYNLGAMSHVAVSFESPEYTADVDAMGTLRLLEAIRFLGLEKKTRFYQASTSELYGLVQE
IPQKETTPFYPRSPYAVAKLYAYWITVNYRESYGMYACNGILFNHESPRRGETFVTRKITRAIANIAQGLE
SCLYLGNMDSLRDWGHAKDYVKMQWMMLQQEQPEDFVIATGVQYSVRQFVEMAAAQLGIKLRFEGTGVEEK
GIVVSVTGHDAPGVKPGDVIIAVDPRYFRPAEVETLLGDPTKAHEKLGWKPEITLREMVSEMVANDLEAAK
KHSLLKSHGYDVAIALES
GDP-L-fucose synthase from Escherichia coli
SEQ ID NO: 19
MSKQRIFIAGHRGMVGSAIRRQLEQRGDVELVLRTRDELNLLDSRAVHDFFASERIDQVYLAAAKVGGIVA
NNTYPADFIYQNMMIESNIIHAAHQNDVNKLLFLGSSCIYPKLAKQPMAESELLQGTLEPTNEPYAIAKIA
GIKLCESYNRQYGRDYRSVMPTNLYGPHDNFHPSNSHVIPALLRRFHEATAQNAPDVVVWGSGTPMREFLH
VDDMVAASIHVMELAHEVWLENTQPMLSHINVGTGVDCTIRELAQTIAKVVGYKGRVVFDASKPDGTPRKL
LDVTRLHQLGWYHEISLEAGLASTYQWFLENQDRFRG
α-1,2-fucosyltransferase from Helicobacter pylori
SEQ ID NO: 20
MAFKVVQICGGLGNQMFQYAFAKSLQKHLNTPVLLDTTSFDWSNRKMQLELFPIDLPYANAKEIAIAKMQH
LPKLVRDALKYIGFDRVSQEIVFEYEPKLLKPSRLTYFFGYFQDPRYFDAISSLIKQTFTLPPPPENNKNN
NKKEEEYQRKLSLILAAKNSVFVHIRRGDYVGIGCQLGIDYQKKALEYMAKRVPNMELFVFCEDLKFTQNL
DLGYPFTDMTTRDKEEEAYWDMLLMQSCKHGIIANSTYSWWAAYLMENPEKIIIGPKHWLFGHENILCKEW
VKIESHFEVKSQKYNA

1. A host cell capable of producing a human milk oligosaccharide (HMO), wherein the host cell is:
(a) deficient in expression of one or more genes encoding an endogenous oxidoreductase;
(b) deficient in activity of one or more endogenous oxidoreductase proteins; and/or
(c) deficient in a level of a reduced form of a reducing sugar.

2. The host cell of claim 1, wherein the endogenous oxidoreductase is an endogenous aldose reductase.

3. The host cell of claim 1 or 2, wherein the host cell is deficient in expression of the one or more genes encoding an endogenous oxidoreductase.

4. The host cell of any one of claims 1-3, wherein the one or more genes comprise gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1.

5. The host cell of any one of claims 1-4, wherein the one or more genes comprise gcy1.

6. The host cell of any one of claims 1-5, wherein the one or more genes comprise gre3.

7. The host cell of any one of claims 1-6, wherein the one or more genes comprise gcy1 and gre3.

8. The host cell of any one of claims 1-7, wherein the one or more genes comprise adh6.

9. The host cell of any one of claims 1-8, wherein the one or more genes comprise sfa1.

10. The host cell of any one of claims 1-9, wherein the host cell is deficient in expression of the one or more genes by virtue of comprising a deletion of the one or more genes from the host cell genome.

11. The host cell of claim 10, wherein the deletion removes one or more nucleotides from the gene, thereby resulting in a missense mutation, nonsense mutation, or frameshift mutation in the gene.

12. The host cell of claim 10 or 11, wherein the deletion removes the one or more genes from the host cell genome in their entirety.

13. The host cell of any one of claims 1-9, wherein the host cell is deficient in expression of the one or more genes by virtue of comprising an inhibitor of expression of the one or more genes.

14. The host cell of claim 13, wherein the inhibitor of expression of the one or more genes is an interfering RNA molecule.

15. The host cell of claim 14, wherein the interfering RNA molecule is a short interfering RNA (siRNA), a short hairpin RNA (shRNA), a micro RNA (miRNA), or an antisense oligonucleotide (ASO).

16. The host cell of any one of claims 1-9, wherein the host cell is deficient in expression of the one or more genes by virtue of comprising a nuclease that catalyzes cleavage of one or more phosphodiester bonds in the one or more genes.

17. The host cell of claim 16, wherein the nuclease is a clustered regularly interspaced short palindromic repeats (CRISPR)-associated protein.

18. The host cell of claim 17, wherein the CRISPR-associated protein is CRISPR associated protein 9 (Cas9) or CRISPR-associated protein 12a (Cas12a).

19. The host cell of claim 16, wherein the nuclease is a transcription activator-like effector nuclease (TALEN), a meganuclease, or a zinc finger nuclease.

20. The host cell of any one of claims 1-19, wherein the host cell is deficient in activity of the one or more endogenous oxidoreductase proteins.

21. The host cell of any one of claims 1-20, wherein the one or more oxidoreductase proteins comprise GCY1, GRE3, ADH6, SFA1, YPR1, GRE2, ARA1, BDH1, BDH2, ADH4, SER3, AAD6, AAD16, ARI1, ADH5, SER33, NRE1, IRC24, IDP2, AAD14, ALD6, ALD3, and/or ADH1.

22. The host cell of any one of claims 1-21, wherein the one or more oxidoreductase proteins comprise GCY1.

23. The host cell of any one of claims 1-22, wherein the one or more oxidoreductase proteins comprise GRE3.

24. The host cell of any one of claims 1-23, wherein the one or more oxidoreductase proteins comprise GCY1 and GRE3.

25. The host cell of any one of claims 1-24, wherein the one or more oxidoreductase proteins comprise ADH6.

26. The host cell of any one of claims 1-25, wherein the one or more oxidoreductase proteins comprise SFA1.

27. The host cell of any one of claims 1-26, wherein the host cell is deficient in activity of the one or more oxidoreductase proteins by virtue of comprising an inhibitor of the one or more oxidoreductase proteins.

28. The host cell of any one of claims 1-27, wherein the host cell is deficient in a level of lactitol, LNnT-alditol, and/or 2′-fucosyllactitol.

29. The host cell of any one of claims 1-28, wherein the host cell comprises one or more heterologous nucleic acids that each, independently, encode one or more enzymes of the biosynthetic pathway of the HMO.

30. The host cell of claim 29, wherein the one or more heterologous nucleic acids encoding one or more enzymes of the biosynthetic pathway of the HMO are integrated into the genome of the host cell.

31. The host cell of claim 29, wherein the one or more heterologous nucleic acids encoding one or more enzymes of the biosynthetic pathway of the HMO are present within one or more plasmids.

32. The host cell of any one of claims 1-31, wherein the HMO is a reducing sugar.

33. The host cell of any one of claims 1-32, wherein the HMO comprises a terminal lactose residue.

34. The host cell of claim 32 or 33, wherein the HMO is lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, or fucosyl-disialyllacto-N-hexaose (FDS-LNH) II.

35. The host cell of claim 34, wherein the HMO is LNnT.

36. The host cell of any one of claims 29-35, wherein the one or more enzymes comprise one or more of a β-1,3-N-acetylglucosaminyltransferase (LgtA), a β-1,4-galactosyltransferase (LgtB), and a UDP-N-acetylglucosamine diphosphorylase.

37. The host cell of claim 36, wherein the one or more enzymes comprise a LgtA.

38. The host cell of claim 37, wherein the LgtA has an amino acid sequence that is at least 85% identical to the amino acid sequence of SEQ ID NO: 1.

39. The host cell of claim 38, wherein the LgtA has an amino acid sequence that is at least 90% identical to the amino acid sequence of SEQ ID NO: 1.

40. The host cell of claim 39, wherein the LgtA has an amino acid sequence that is at least 95% identical to the amino acid sequence of SEQ ID NO: 1.

41. The host cell of claim 40, wherein the LgtA has the amino acid sequence of SEQ ID NO: 1.

42. The host cell of claim 36 or 37, wherein the LgtA comprises one or more amino acid substitutions or deletions relative to the amino acid sequence of SEQ ID NO: 1.

43. The host cell of claim 42, wherein the LgtA has an amino acid sequence that is from about 85% to about 99.7% identical to the amino acid sequence of SEQ ID NO: 1, optionally wherein the LgtA has an amino acid sequence that is from about 90% to about 99.7% identical to the amino acid sequence of SEQ ID NO: 1, optionally wherein the LgtA has an amino acid sequence that is from about 95% to about 99.7% identical to the amino acid sequence of SEQ ID NO: 1.

44. The host cell of claim 42 or 43, wherein the LgtA has an amino acid sequence that differs from the amino acid sequence of SEQ ID NO: 1 only by way of (i) the one or more amino acid substitutions or deletions and, optionally, (ii) one or more additional, conservative amino acid substitutions.

45. The host cell of claim 44, wherein the LgtA has an amino acid sequence that differs from the amino acid sequence of SEQ ID NO: 1 only by way of the one or more amino acid substitutions or deletions.

46. The host cell of any one of claims 37 and 42-45, wherein the LgtA has an amino acid sequence that is at least 85% identical to the amino acid sequence of any one of SEQ ID NO: 2-13.

47. The host cell of claim 46, wherein the LgtA has an amino acid sequence that is at least 90% identical to the amino acid sequence of any one of SEQ ID NO: 2-13.

48. The host cell of claim 47, wherein the LgtA has an amino acid sequence that is at least 95% identical to the amino acid sequence of any one of SEQ ID NO: 2-13.

49. The host cell of claim 48, wherein the LgtA has the amino acid sequence of any one of SEQ ID NO: 2-13.

50. The host cell of any one of claims 36-49, wherein the one or more enzymes comprise a LgtB.

51. The host cell of claim 50, wherein the LgtB has an amino acid sequence that is at least 85% identical to the amino acid sequence of SEQ ID NO: 15.

52. The host cell of claim 51, wherein the LgtB has an amino acid sequence that is at least 90% identical to the amino acid sequence of SEQ ID NO: 15.

53. The host cell of claim 52, wherein the LgtB has an amino acid sequence that is at least 95% identical to the amino acid sequence of SEQ ID NO: 15.

54. The host cell of claim 53, wherein the LgtB has the amino acid sequence of SEQ ID NO: 15.

55. The host cell of claim 50, wherein the LgtB has an amino acid sequence that is at least 85% identical to the amino acid sequence of SEQ ID NO: 16.

56. The host cell of claim 55, wherein the LgtB has an amino acid sequence that is at least 90% identical to the amino acid sequence of SEQ ID NO: 16.

57. The host cell of claim 56, wherein the LgtB has an amino acid sequence that is at least 95% identical to the amino acid sequence of SEQ ID NO: 16.

58. The host cell of claim 57, wherein the LgtB has the amino acid sequence of SEQ ID NO: 16.

59. The host cell of claim 34, wherein the HMO is 2′-FL.

60. The host cell of any one of claims 29-34 and 59, wherein the one or more enzymes comprise one or more of a GDP-mannose 4,6-dehydratase, a GDP-L-fucose synthase, an α-1,2-fucosyltransferase, and optionally a fucosidase.

61. The host cell of claim 60, wherein the one or more enzymes comprise a GDP-mannose 4,6-dehydratase.

62. The host cell of claim 61, wherein the GDP-mannose 4,6-dehydratase has an amino acid sequence that is at least 85% identical to the amino acid sequence of SEQ ID NO: 18.

63. The host cell of claim 62, wherein the GDP-mannose 4,6-dehydratase has an amino acid sequence that is at least 90% identical to the amino acid sequence of SEQ ID NO: 18.

64. The host cell of claim 63, wherein the GDP-mannose 4,6-dehydratase has an amino acid sequence that is at least 95% identical to the amino acid sequence of SEQ ID NO: 18.

65. The host cell of claim 64, wherein the GDP-mannose 4,6-dehydratase has the amino acid sequence of SEQ ID NO: 18.

66. The host cell of any one of claims 60-65, wherein the one or more enzymes comprise a GDP-L-fucose synthase.

67. The host cell of claim 66, wherein the GDP-L-fucose synthase has an amino acid sequence that is at least 85% identical to the amino acid sequence of SEQ ID NO: 19.

68. The host cell of claim 67, wherein the GDP-L-fucose synthase has an amino acid sequence that is at least 90% identical to the amino acid sequence of SEQ ID NO: 19.

69. The host cell of claim 68, wherein the GDP-L-fucose synthase has an amino acid sequence that is at least 95% identical to the amino acid sequence of SEQ ID NO: 19.

70. The host cell of claim 69, wherein the GDP-L-fucose synthase has the amino acid sequence of SEQ ID NO: 19.

71. The host cell of any one of claims 60-70, wherein the one or more enzymes comprise an a-1,2-fucosyltransferase.

72. The host cell of claim 71, wherein the α-1,2-fucosyltransferase has an amino acid sequence that is at least 85% identical to the amino acid sequence of SEQ ID NO: 20.

73. The host cell of claim 72, wherein the α-1,2-fucosyltransferase has an amino acid sequence that is at least 90% identical to the amino acid sequence of SEQ ID NO: 20.

74. The host cell of claim 73, wherein the α-1,2-fucosyltransferase has an amino acid sequence that is at least 95% identical to the amino acid sequence of SEQ ID NO: 20.

75. The host cell of claim 74, wherein the α-1,2-fucosyltransferase has the amino acid sequence of SEQ ID NO: 20.

76. The host cell of any one of claims 29-75, wherein the one or more heterologous nucleic acids further encode a protein that transports lactose into the host cell.

77. The host cell of claim 76, wherein the protein that transports lactose into the host cell is a lactose permease.

78. The host cell of claim 77, wherein the lactose permease has an amino acid sequence that is at least 85% identical to the amino acid sequence of SEQ ID NO: 14.

79. The host cell of claim 78, wherein the lactose permease has an amino acid sequence that is at least 90% identical to the amino acid sequence of SEQ ID NO: 14.

80. The host cell of claim 79, wherein the lactose permease has an amino acid sequence that is at least 95% identical to the amino acid sequence of SEQ ID NO: 14.

81. The host cell of claim 80, wherein the lactose permease has the amino acid sequence of SEQ ID NO: 14.

82. The host cell of claim 76, wherein the protein that transports lactose into the cell is an active transporter.

83. The host cell of any one of claims 29-82, wherein expression of the one or more heterologous nucleic acids is driven by an inducible promoter or is negatively regulated by the activity of a promoter that is responsive to a small molecule.

84. The host cell of any one of claims 1-83, wherein the host cell is a yeast cell.

85. The host cell of claim 84, wherein the yeast cell is a Saccharomyces sp. cell or a Kluveromyces sp. cell.

86. The host cell of claim 85, wherein the yeast cell is a Saccharomyces cerevisiae cell.

87. The host cell of claim 85, wherein the yeast cell is a Kluveromyces marxianus cell.

88. A method of producing a HMO, the method comprising culturing a population of host cells of any one of claims 1-87 in a culture medium under conditions suitable for the host cells to produce the HMO.

89. The method of claim 88, wherein the HMO is a reducing sugar.

90. The method of claim 88 or 89, wherein the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

91. The method of claim 90, wherein the HMO is LNnT.

92. The method of claim 90, wherein the HMO is 2′-FL.

93. A fermentation composition comprising (i) a population of host cells of any one of claims 1-87 and (ii) a culture medium comprising a HMO produced from the host cells.

94. The fermentation composition of claim 93, wherein the HMO is a reducing sugar.

95. The fermentation composition of claim 94, wherein the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

96. The fermentation composition of claim 95, wherein the HMO is LNnT.

97. The fermentation composition of claim 95, wherein the HMO is 2′-FL.

98. A method of genetically modifying a host cell to produce a HMO, the method comprising:
a. (i) rendering the host cell deficient in expression of one or more genes encoding an endogenous oxidoreductase and (ii) introducing one or more heterologous nucleic acids that each, independently, encode one or more enzymes of the biosynthetic pathway of the HMO into the host cell; or
b. rendering the host cell deficient in expression of one or more genes encoding an endogenous oxidoreductase, wherein the host cell comprises one or more heterologous nucleic acids that each, independently, encode one or more enzymes of the biosynthetic pathway of the HMO; or
c. introducing one or more heterologous nucleic acids that each, independently, encode one or more enzymes of the biosynthetic pathway of the HMO into the host cell, wherein the host cell has previously been rendered deficient in expression of one or more genes encoding an endogenous oxidoreductase,
wherein the one or more heterologous nucleic acids, together with the endogenous genes present in the host cell, collectively encode the entirety of the enzymes of the biosynthetic pathway of the HMO.

99. The method of claim 98, wherein the endogenous oxidoreductase is an endogenous aldose reductase.

100. The method of claim 98 or 99, wherein the one or more genes encoding an endogenous oxidoreductase comprise gcy1, gre3, adh6, sfa1, ypr1, gre2, ara1, bdh1, bdh2, adh4, ser3, aad6, aad16, ari1, adh5, ser33, nre1, irc24, idp2, aad14, ald6, ald3, and/or adh1.

101. The method of any one of claims 98-100, wherein the one or more genes encoding an endogenous oxidoreductase comprise gcy1.

102. The method of any one of claims 98-101, wherein the one or more genes encoding an endogenous oxidoreductase comprise gre3.

103. The method of any one of claims 98-102, wherein the one or more genes encoding an endogenous oxidoreductase comprise gcy1 and gre3.

104. The method of any one of claims 98-103, wherein the one or more genes encoding an endogenous oxidoreductase comprise adh6.

105. The method of any one of claims 98-104, wherein the one or more genes encoding an endogenous oxidoreductase comprise sfa1.

106. The method of any one of claims 98-105, wherein the host cell is rendered deficient in expression of the one or more genes encoding an endogenous oxidoreductase by way of a deletion of the one or more genes from the host cell genome.

107. The method of claim 106, wherein the deletion removes one or more nucleotides from the gene, thereby resulting in a missense mutation, nonsense mutation, or frameshift mutation in the gene.

108. The method of claim 106 or 107, wherein the deletion removes the one or more genes from the host cell genome in their entirety.

109. The method of any one of claims 98-105, wherein the host cell is rendered deficient in expression of the one or more genes encoding an endogenous oxidoreductase by introducing into the host cell an inhibitor of expression of the one or more genes.

110. The method of claim 109, wherein the inhibitor of expression of the one or more genes is an interfering RNA molecule.

111. The method of claim 110, wherein the interfering RNA molecule is a siRNA, a shRNA, a miRNA, or an ASO.

112. The method of any one of claims 98-105, wherein the host cell is rendered deficient in expression of the one or more genes encoding an endogenous oxidoreductase by introducing into the host cell a nuclease that catalyzes cleavage of one or more phosphodiester bonds in the one or more genes.

113. The method of claim 112, wherein the nuclease is a CRISPR-associated protein.

114. The method of claim 113, wherein the CRISPR-associated protein is CRISPR associated protein 9 (Cas9) or CRISPR-associated protein 12a (Cas12a).

115. The method of claim 112, wherein the nuclease is a TALEN, a meganuclease, or a zinc finger nuclease.

116. The method of any one of claims 98-115, wherein the one or more heterologous nucleic acids encoding one or more enzymes of the biosynthetic pathway of the HMO are integrated into the genome of the host cell.

117. The method of any one of claims 98-115, wherein the one or more heterologous nucleic acids encoding one or more enzymes of the biosynthetic pathway of the HMO are present within one or more plasmids.

118. The method of any one of claims 98-117, wherein the HMO is a reducing sugar.

119. The method of any one of claims 98-118, wherein the HMO comprises a terminal lactose residue.

120. The method of claim 118 or 119, wherein the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

121. The method of claim 120, wherein the HMO is LNnT.

122. The method of any one of claims 98-121, wherein the one or more heterologous nucleic acids encode a β-1,3-N-acetylglucosaminyltransferase (LgtA), a β-1,4-galactosyltransferase (LgtB), and/or a UDP-N-acetylglucosamine diphosphorylase.

123. The method of claim 122, wherein the one or more heterologous nucleic acids encode a LgtA.

124. The method of claim 123, wherein the LgtA has an amino acid sequence that is at least 85% identical to the amino acid sequence of SEQ ID NO: 1.

125. The method of claim 124, wherein the LgtA has an amino acid sequence that is at least 90% identical to the amino acid sequence of SEQ ID NO: 1.

126. The method of claim 125, wherein the LgtA has an amino acid sequence that is at least 95% identical to the amino acid sequence of SEQ ID NO: 1.

127. The method of claim 126, wherein the LgtA has the amino acid sequence of SEQ ID NO: 1.

128. The method of claim 122 or 123, wherein the LgtA comprises one or more amino acid substitutions or deletions relative to the amino acid sequence of SEQ ID NO: 1.

129. The method of claim 128, wherein the LgtA has an amino acid sequence that is from about 85% to about 99.7% identical to the amino acid sequence of SEQ ID NO: 1, optionally wherein the LgtA has an amino acid sequence that is from about 90% to about 99.7% identical to the amino acid sequence of SEQ ID NO: 1, optionally wherein the LgtA has an amino acid sequence that is from about 95% to about 99.7% identical to the amino acid sequence of SEQ ID NO: 1.

130. The method of claim 128 or 129, wherein the LgtA has an amino acid sequence that differs from the amino acid sequence of SEQ ID NO: 1 only by way of (i) the one or more amino acid substitutions or deletions and, optionally, (ii) one or more additional, conservative amino acid substitutions.

131. The method of claim 130, wherein the LgtA has an amino acid sequence that differs from the amino acid sequence of SEQ ID NO: 1 only by way of the one or more amino acid substitutions or deletions.

132. The method of any one of claims 123 and 128-131, wherein the LgtA has an amino acid sequence that is at least 85% identical to the amino acid sequence of any one of SEQ ID NO: 2-13.

133. The method of claim 132, wherein the LgtA has an amino acid sequence that is at least 90% identical to the amino acid sequence of any one of SEQ ID NO: 2-13.

134. The method of claim 133, wherein the LgtA has an amino acid sequence that is at least 95% identical to the amino acid sequence of any one of SEQ ID NO: 2-13.

135. The method of claim 134, wherein the LgtA has the amino acid sequence of any one of SEQ ID NO: 2-13.

136. The method of any one of claims 122-135, wherein the one or more heterologous nucleic acids encode a LgtB.

137. The method of claim 136, wherein the LgtB has an amino acid sequence that is at least 85% identical to the amino acid sequence of SEQ ID NO: 15.

138. The method of claim 137, wherein the LgtB has an amino acid sequence that is at least 90% identical to the amino acid sequence of SEQ ID NO: 15.

139. The method of claim 138, wherein the LgtB has an amino acid sequence that is at least 95% identical to the amino acid sequence of SEQ ID NO: 15.

140. The method of claim 139, wherein the LgtB has the amino acid sequence of SEQ ID NO: 15.

141. The method of claim 136, wherein the LgtB has an amino acid sequence that is at least 85% identical to the amino acid sequence of SEQ ID NO: 16.

142. The method of claim 141, wherein the LgtB has an amino acid sequence that is at least 90% identical to the amino acid sequence of SEQ ID NO: 16.

143. The method of claim 142, wherein the LgtB has an amino acid sequence that is at least 95% identical to the amino acid sequence of SEQ ID NO: 16.

144. The method of claim 143, wherein the LgtB has the amino acid sequence of SEQ ID NO: 16.

145. The method of claim 120, wherein the HMO is 2′-FL.

146. The method of any one of claims 98-120 and 145, wherein the one or more heterologous nucleic acids encode a GDP-mannose 4,6-dehydratase, a GDP-L-fucose synthase, and/or an α-1,2-fucosyltransferase, optionally in combination with a fucosidase.

147. The method of claim 146, wherein the one or more heterologous nucleic acids encode a GDP-mannose 4,6-dehydratase.

148. The method of claim 147, wherein the GDP-mannose 4,6-dehydratase has an amino acid sequence that is at least 85% identical to the amino acid sequence of SEQ ID NO: 18.

149. The method of claim 148, wherein the GDP-mannose 4,6-dehydratase has an amino acid sequence that is at least 90% identical to the amino acid sequence of SEQ ID NO: 18.

150. The method of claim 149, wherein the GDP-mannose 4,6-dehydratase has an amino acid sequence that is at least 95% identical to the amino acid sequence of SEQ ID NO: 18.

151. The method of claim 150, wherein the GDP-mannose 4,6-dehydratase has the amino acid sequence of SEQ ID NO: 18.

152. The method of any one of claims 146-151, wherein the one or more heterologous nucleic acids encode a GDP-L-fucose synthase.

153. The method of claim 152, wherein the GDP-L-fucose synthase has an amino acid sequence that is at least 85% identical to the amino acid sequence of SEQ ID NO: 19.

154. The method of claim 153, wherein the GDP-L-fucose synthase has an amino acid sequence that is at least 90% identical to the amino acid sequence of SEQ ID NO: 19.

155. The method of claim 154, wherein the GDP-L-fucose synthase has an amino acid sequence that is at least 95% identical to the amino acid sequence of SEQ ID NO: 19.

156. The method of claim 155, wherein the GDP-L-fucose synthase has the amino acid sequence of SEQ ID NO: 19.

157. The method of any one of claims 146-156, wherein the one or more heterologous nucleic acids encode an α-1,2-fucosyltransferase.

158. The method of claim 157, wherein the α-1,2-fucosyltransferase has an amino acid sequence that is at least 85% identical to the amino acid sequence of SEQ ID NO: 20.

159. The method of claim 158, wherein the α-1,2-fucosyltransferase has an amino acid sequence that is at least 90% identical to the amino acid sequence of SEQ ID NO: 20.

160. The method of claim 159, wherein the α-1,2-fucosyltransferase has an amino acid sequence that is at least 95% identical to the amino acid sequence of SEQ ID NO: 20.

161. The method of claim 160, wherein the α-1,2-fucosyltransferase has the amino acid sequence of SEQ ID NO: 20.

162. The method of any one of claims 98-161, wherein the one or more heterologous nucleic acids encode a protein that transports lactose into the host cell.

163. The method of claim 162, wherein the protein that transports lactose into the host cell is a lactose permease.

164. The method of claim 163, wherein the lactose permease has an amino acid sequence that is at least 85% identical to the amino acid sequence of SEQ ID NO: 14.

165. The method of claim 164, wherein the lactose permease has an amino acid sequence that is at least 90% identical to the amino acid sequence of SEQ ID NO: 14.

166. The method of claim 165, wherein the lactose permease has an amino acid sequence that is at least 95% identical to the amino acid sequence of SEQ ID NO: 14.

167. The method of claim 166, wherein the lactose permease has the amino acid sequence of SEQ ID NO: 14.

168. The method of claim 162, wherein the protein that transports lactose into the cell is an active transporter.

169. The method of any one of claims 98-168, wherein the host cell is a yeast cell.

170. The method of claim 169, wherein the yeast cell is a Saccharomyces sp. cell or a Kluveromyces sp. cell.

171. The method of claim 170, wherein the yeast cell is a Saccharomyces cerevisiae cell.

172. The method of claim 170, wherein the yeast cell is a Kluveromyces marxianus cell.

173. A method of producing an infant formula, the method comprising:
a. culturing a population of host cells of any one of claims 1-87 in a culture medium under conditions suitable for the host cells to produce the HMO;
b. extracting the HMO; and
c. formulating the HMO for administration to an infant.

174. The method of claim 173, wherein the HMO is a reducing sugar.

175. The method of claim 173 or 174, wherein the HMO comprises a terminal lactose residue.

176. The method of any one of claims 173-175, wherein the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

177. The method of any one of claims 173-176, wherein the infant formula comprises LNnT.

178. The method of any one of claims 173-177, wherein the infant formula comprises 2′-FL.

179. An infant formula produced by the method of any one of claims 173-178.


SEQUENCE LISTING (20 sequences)

SEQ ID NO: 1 - protein, 348 aa, Neisseria meningitidis
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 2 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTDRNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 3 - protein, 319 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPMPVPG

SEQ ID NO: 4 - protein, 300 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPNEDFRKS

SEQ ID NO: 5 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 6 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSHYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 7 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEAPVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 8 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTPIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 9 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNGTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 10 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLITSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 11 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYVFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 12 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVVSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 13 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFNYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 14 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 15 - protein, 282 aa, Pasteurella multocida
MSGEHYVISLSSAVERRQHIRNQFSQKNIPFQFFDAISPSPLLDQLVLQFFPRLADSSLTGGEKACFMSHLSLWHKCVEENLPYIVVFEDDIVLGKDADKFLIGDEWLFSRFDPEEIFIIRLETFLQKVVCESTHIAPYTHRDFLSLKSAHFGTAGYVISQGAAKFLLDIFKNISNEHIAPIDELIFNQFLVKNSFNVYQLSPAICVQELQLNNESSALQSQLELERNKFRNKKSEELKRNRKNFIEKFIYILKKPKRMLDNNKRKREESKIENDKMIIEFK

SEQ ID NO: 16 - protein, 279 aa, Neisseria gonorrhoeae
MQNHVISLASAAERRAHIAATFGSRGIPFQFFDALMPSERLERAMAELVPGLSAHPYLSGVEKACFMSHAVLWEQALDEGVPYIAVFEDDVLLGEGAEQFLAEDTWLQERFDPDSAFVVRLETMFMHVLTSPSGVADYGGRAFPLLESEHCGTAGYIISRKAMRFFLDRFAVLPPERLHPVDLMMFGNPDDREGMPVCQLNPALCAQELHYAKFHDQNSALGSLIEHDRRLNRKQQWRDSPANTFKHRLIRALTKIGREREKRRQRREQLIGKIIVPFQ

SEQ ID NO: 17 - protein, 279 aa, Kluyveromyces lactis; identical to SEQ ID NO: 16

SEQ ID NO: 18 - protein, 373 aa, Escherichia coli [sequence omitted]

SEQ ID NO: 19 - protein, 321 aa, Escherichia coli
MSKQRIFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMVAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 20 - protein, 300 aa, Helicobacter pylori
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYQRKLSLILAAKNSVFVHIRRGDYVGIGCQLGIDYQKKALEYMAKRVPNMELFVFCEDLKFTQNLDLGYPFTDMTTRDKEEEAYWDMLLMQSCKHGIIANSTYSWWAAYLMENPEKIIIGPKHWLFGHENILCKEWVKIESHFEVKSQKYNA